(19) United States
(12) Patent Application Publication
(10) Pub. No.: US 2025/0345351 A1
(43) Pub. Date: Nov. 13, 2025

(54) OLIGOSACCHARIDE COMPOSITIONS AND METHODS OF USE

(71) Applicant: DSM NUTRITIONAL PRODUCTS, LLC, Parsippany, NJ (US)

(72) Inventors: Jeffrey MEISNER, Lexington, MA (US); Christopher Matthew LIU, Somerville, MA (US); Madeline ROSINI, Lexington, MA (US); Max HECHT, Lexington, MA (US); Eric HUMPHRIES, Lexington, MA (US); Adarsh JOSE, Lexington, MA (US); Johan VAN HYLCKAMA VLIEG, Lexington, MA (US); Mark DOWLING, Lexington, MA (US); Mark WINGERTZAHN, Lexington, MA (US); Jackson LEE, Lexington, MA (US)

(73) Assignee: DSM Nutritional Products, LLC, Parsippany, NJ (US)

(21) Appl. No.: 18/027,732

(22) PCT Filed: Sep. 24, 2021
(86) PCT No.: PCT/US2021/052098

§ 371 Date: Mar. 22, 2023

Related U.S. Application Data
(60) Provisional application No. 63/083,796, filed on Sep. 25, 2020.

(51) Int. Cl. A61K 31/702; A61P 1/00; A61P 1/04

(52) U.S. Cl. CPC A61K 31/702; A61P 1/00; A61P 1/04

(57) ABSTRACT

Aspects of the disclosure relate to oligosaccharide compositions and methods of making the same. Also provided are methods of using oligosaccharide compositions as microbiome metabolic therapies for decreasing inflammation for the treatment of inflammatory and immune disorders and diseases.

### FIELD OF THE INVENTION

The present disclosure relates to oligosaccharide compositions and uses thereof.

### BACKGROUND OF THE INVENTION

Maintaining or restoring human health faces a large number of challenges many of which result from the lack of effective treatment options. Specifically, inflammatory bowel disease (IBD), which includes ulcerative colitis (UC) and Chron's disease (CD) and affects approximately 3 million people in the United States, is a chronic relapsing immunologically mediated disease of the intestine that has a lack of effective treatment options. The pathogenesis of IBD is poorly understood, but is thought to be caused by an interplay of genetic, environmental, intestinal barrier, and immunologic factors that alter gut homeostasis and trigger inflammation in susceptible individuals. There is a continued need for novel therapies and treatment regimens for diseases and disorders such as IBD.

### SUMMARY OF THE INVENTION

According to some aspects, provided herein are microbiome metabolic therapies utilizing oligosaccharide compositions that are useful for driving functional outputs of the gut microbiome organ, e.g., to treat disease. Some aspects of the disclosure relate to a recognition that oligosaccharide compositions are useful for increasing levels of short chain fatty acids (SCFAs) such as butyrate, acetate, and/or propionate in a subject and for promoting the growth and abundance of commensal bacteria relative to pathogenic bacteria, two functional outcomes which are useful for treating a number of inflammatory and immune disorders, including autoimmune and allergic disorders (e.g., chronic inflammatory disorders, e.g., inflammatory bowel diseases, e.g., ulcerative colitis (UC) and Crohn's disease (CD)). Thus, in some aspects described herein, oligosaccharide compositions of the disclosure are effective in treating inflammatory and immune disorders, including ulcerative colitis.

Provided herein, in some aspects, an oligosaccharide composition comprising a plurality of oligosaccharides, the plurality of oligosaccharides being characterized by a multiplicity-edited gradient-enhanced ¹H-¹³C heteronuclear single quantum correlation (HSQC) NMR spectrum comprising one or more of signals 2, 3, and 11 of the following table,
wherein the area under the curve (AUC) for each of signals 1-11 is determined by obtaining the integration of integral regions defined by an 1H center position and an ¹³C center position using an elliptical shape, and wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of signals 1-11) |
| 1 | 5.04 | 108.38 | 2.05-7.82 |
| 2 | 5.02 | 101.68 | 0.34-2.01 |
| 3 | 4.98 | 98.99 | 7.28-25.71 |
| 4 | 4.81 | 101.2 | 0.29-2.32 |
| 5 | 4.6 | 104.96 | 0.23-3.87 |
| 6 | 4.45 | 103.84 | 6.52-15.87 |
| 7 | 4.19 | 82.1 | 3.61-5.43 |
| 8 | 4.14 | 76.91 | 6.03-15.36 |
| 9 | 4.11 | 81.72 | 11.13-20.24 |
| 10 | 4.06 | 77.25 | 13.96-28.58 |
| 11 | 3.98 | 83.52 | 7.93-12.69 |

In some embodiments, the oligosaccharide composition comprises 2 or 3 of signals 2, 3, and 11, wherein signal 2 has an AUC (% of total areas of signals 1-11) in the range of 0.34-2.01, signal 3 has an AUC (% of total areas of signals 1-11) in the range of 7.28-25.71, and signal 11 has an AUC (% of total areas of signals 1-11) in the range of 7.93-12.69.

In some embodiments, the oligosaccharide composition comprises 2 or 3 of signals 2, 3, and 11, wherein signal 2 has an AUC (% of total areas of signals 1-11) in the range of 0.68-1.68, signal 3 has an AUC (% of total areas of signals 1-11) in the range of 10.97-22.02, and signal 11 has an AUC (% of total areas of signals 1-11) in the range of 8.88-11.74.

In some embodiments, the oligosaccharide composition further comprises signal 5, wherein signal 5 has an AUC (% of total areas of signals 1-11) in the range of 0.23-3.87. In some embodiments, the oligosaccharide composition further comprises signal 5, wherein signal 5 has an AUC (% of total areas of signals 1-11) in the range of 0.96-3.14.

In some embodiments, the oligosaccharide composition further comprises one or more (e.g., 1, 2, 3, 4, 5, 6, 7, 8, 9, or 10) of signals 1, 4, 6, 7, 8, 9, and 10, wherein at least signals 1, 4, 6, 7, 8, 9, and 10 are defined as follows:

| | Center Position | | |
|---|---|---|---|
| | (ppm) | | Area under the curve (AUC) |
| Signal | ¹H | ¹³C | (% of total areas of signals 1-11) |
| 1 | 5.04 | 108.38 | 2.05-7.82 |
| 2 | 5.02 | 101.68 | 0.34-2.01 |
| 3 | 4.98 | 98.99 | 7.28-25.71 |
| 4 | 4.81 | 101.2 | 0.29-2.32 |
| 5 | 4.6 | 104.96 | 0.23-3.87 |
| 6 | 4.45 | 103.84 | 6.52-15.87 |
| 7 | 4.19 | 82.1 | 3.61-5.43 |
| 8 | 4.14 | 76.91 | 6.03-15.36 |
| 9 | 4.11 | 81.72 | 11.13-20.24 |
| 10 | 4.06 | 77.25 | 13.96-28.58 |
| 11 | 3.98 | 83.52 | 7.93-12.69 |

In some embodiments, at least one of signals 1-11 is defined as follows:

| | Center Position | | |
|---|---|---|---|
| | (ppm) | | Area under the curve (AUC) |
| Signal | ¹H | ¹³C | (% of total areas of signals 1-11) |
| 1 | 5.04 | 108.38 | 3.20-6.67 |
| 2 | 5.02 | 101.68 | 0.68-1.68 |
| 3 | 4.98 | 98.99 | 10.97-22.02 |
| 4 | 4.81 | 101.2 | 0.70-1.92 |
| 5 | 4.6 | 104.96 | 0.96-3.14 |
| 6 | 4.45 | 103.84 | 8.39-14.00 |
| 7 | 4.19 | 82.1 | 3.97-5.07 |
| 8 | 4.14 | 76.91 | 7.89-13.50 |
| 9 | 4.11 | 81.72 | 12.95-18.42 |
| 10 | 4.06 | 77.25 | 16.88-25.66 |
| 11 | 3.98 | 83.52 | 8.88-11.74 |

In some aspects herein is an oligosaccharide composition comprising a plurality of oligosaccharides, the plurality of oligosaccharides being characterized by a multiplicity-edited gradient-enhanced ¹H-¹³C heteronuclear single quantum correlation (HSQC) NMR spectrum comprising one or more of signals 2, 3, and 11 of the following table, wherein the area under the curve (AUC) for each of signals 1-11 is determined by obtaining the integration of integral regions defined by an 1H center position and an 13C center position using an elliptical shape, and wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of signals 1-11) |
| 1 | 5.04 | 108.38 | 4.46-6.18 |
| 2 | 5.02 | 101.68 | 0.77-1.70 |
| 3 | 4.98 | 98.99 | 10.52-22.14 |
| 4 | 4.81 | 101.2 | 0.98-1.85 |
| 5 | 4.6 | 104.96 | 0.77-3.24 |
| 6 | 4.45 | 103.84 | 8.06-14.63 |
| 7 | 4.19 | 82.1 | 3.98-5.01 |
| 8 | 4.14 | 76.91 | 7.88-12.89 |
| 9 | 4.11 | 81.72 | 12.78-18.67 |
| 10 | 4.06 | 77.25 | 15.75-26.28 |
| 11 | 3.98 | 83.52 | 9.14-11.59 |

In some embodiments, the oligosaccharide composition comprises 2 or 3 of signals 2, 3, and 11, wherein signal 2 has an AUC (% of total areas of signals 1-11) in the range of 0.77-1.70, signal 3 has an AUC (% of total areas of signals 1-11) in the range of 10.52-22.14, and signal 11 has an AUC (% of total areas of signals 1-11) in the range of 9.14-11.59.

In some embodiments, the oligosaccharide composition comprises 2 or 3 of signals 2, 3, and 11, wherein signal 2 has an AUC (% of total areas of signals 1-11) in the range of 1.05-1.45, signal 3 has an AUC (% of total areas of signals 1-11) in the range of 12.94-18.90, and signal 11 has an AUC (% of total areas of signals 1-11) in the range of 9.73-10.99.

In some embodiments, the oligosaccharide composition further comprises signal 5, wherein signal 5 has an AUC (% of total areas of signals 1-11) in the range of 0.77-3.24.

In some embodiments, the oligosaccharide composition further comprises signal 5, wherein signal 5 has an AUC (% of total areas of signals 1-11) in the range of 1.26-2.42.

In some embodiments, the oligosaccharide composition further comprises one or more (e.g., 1, 2, 3, 4, 5, 6, 7, 8, 9, or 10) of signals 1, 4, 6, 7, 8, 9, and 10, wherein at least signals 1, 4, 6, 7, 8, 9, and 10 are defined as follows:

| | Center Position (ppm) | | Area under the curve (AUC) |
|---|---|---|---|
| Signal | ¹H | ¹³C | (% of total areas of signals 1-11) |
| 1 | 5.04 | 108.38 | 4.46-6.18 |
| 2 | 5.02 | 101.68 | 0.77-1.70 |
| 3 | 4.98 | 98.99 | 10.52-22.14 |
| 4 | 4.81 | 101.2 | 0.98-1.85 |
| 5 | 4.6 | 104.96 | 0.77-3.24 |
| 6 | 4.45 | 103.84 | 8.06-14.63 |
| 7 | 4.19 | 82.1 | 3.98-5.01 |
| 8 | 4.14 | 76.91 | 7.88-12.89 |
| 9 | 4.11 | 81.72 | 12.78-18.67 |
| 10 | 4.06 | 77.25 | 15.75-26.28 |
| 11 | 3.98 | 83.52 | 9.14-11.59 |

In some embodiments, the oligosaccharide composition comprises at least one of signals 1-11 of the oligosaccharide composition is defined as follows:

| | Center Position (ppm) | | Area under the curve (AUC) |
|---|---|---|---|
| Signal | ¹H | ¹³C | (% of total areas of signals 1-11) |
| 1 | 5.04 | 108.38 | 4.72-5.66 |
| 2 | 5.02 | 101.68 | 1.05-1.45 |
| 3 | 4.98 | 98.99 | 12.94-18.90 |
| 4 | 4.81 | 101.2 | 1.14-1.62 |
| 5 | 4.6 | 104.96 | 1.26-2.42 |
| 6 | 4.45 | 103.84 | 9.43-12.60 |
| 7 | 4.19 | 82.1 | 4.32-4.85 |
| 8 | 4.14 | 76.91 | 9.10-11.96 |
| 9 | 4.11 | 81.72 | 14.20-17.37 |
| 10 | 4.06 | 77.25 | 19.38-24.58 |
| 11 | 3.98 | 83.52 | 9.73-10.99 |

In some embodiments, the integral regions defined by an ¹H center position and an ¹³C center position of signals 1-11 are further defined as follows:

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | To | Position | from | to |
| 1 | 5.04 | 5.08 | 5.01 | 108.38 | 108.06 | 108.70 |
| 2 | 5.02 | 5.04 | 5.00 | 101.68 | 101.47 | 101.90 |
| 3 | 4.98 | 5.02 | 4.94 | 98.99 | 98.40 | 99.58 |
| 4 | 4.81 | 4.82 | 4.79 | 101.2 | 100.99 | 101.42 |
| 5 | 4.6 | 4.62 | 4.58 | 104.96 | 104.74 | 105.17 |
| 6 | 4.45 | 4.50 | 4.40 | 103.84 | 103.46 | 104.21 |
| 7 | 4.19 | 4.21 | 4.16 | 82.1 | 81.72 | 82.48 |
| 8 | 4.14 | 4.19 | 4.10 | 76.91 | 76.43 | 77.39 |
| 9 | 4.11 | 4.17 | 4.05 | 81.72 | 81.35 | 82.10 |
| 10 | 4.06 | 4.11 | 4.02 | 77.25 | 76.61 | 77.90 |
| 11 | 3.98 | 4.03 | 3.93 | 83.52 | 83.09 | 83.95 |

In some embodiments, the NMR spectrum is obtained by subjecting a sample of the composition to a multiplicity-edited gradient-enhanced ¹H-¹³C heteronuclear single quantum coherence (HSQC) experiment (e.g., in an NMR instrument operating at 500 MHz) using an echo-antiecho scheme for coherence selection using the following pulse sequence diagram, acquisition parameters and processing parameters:

In some embodiments, the NMR spectrum is obtained by subjecting a sample of the composition to a multiplicity-edited gradient-enhanced ¹H-¹³C heteronuclear single quantum coherence (HSQC) experiment (e.g., in an NMR instrument operating at 600 MHz) using an echo-antiecho scheme for coherence selection using the following pulse sequence diagram, acquisition parameters and processing parameters:

In some embodiments, the NMR spectrum is obtained by subjecting a sample of the oligosaccharide composition to HSQC NMR, wherein the sample is dissolved in D₂O. In some embodiments, the oligosaccharide composition has been subjected to a de-monomerization procedure.

In some embodiments, the oligosaccharide composition comprises less than 10% monomer. In some embodiments, the oligosaccharide composition comprises less than 5% monomer. In some embodiments, the oligosaccharide composition comprises less than 2% monomer.

In some embodiments, the oligosaccharide composition comprises a plurality of oligosaccharides that consist essentially of Formula (I):

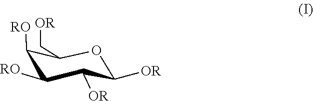

wherein R in Formula (I) is independently selected from hydrogen, and Formulae (Ia), (Ib), (Ic), (Id):

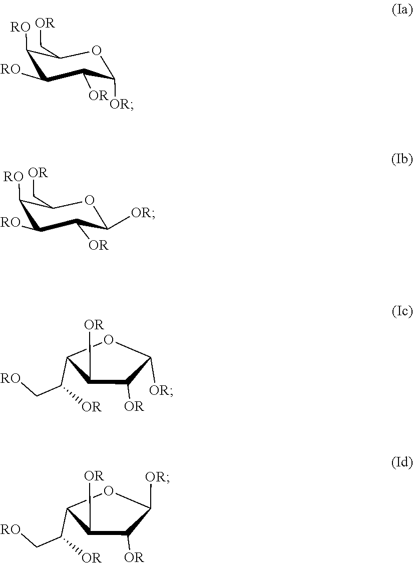

wherein R in Formulae (Ia), (Ib), (Ic), and (Id) is independently defined as above in Formula (I).

In some aspects, provided herein is an oligosaccharide composition comprising a plurality of oligosaccharides, each oligosaccharide comprising a plurality of monomer radicals; the plurality of oligosaccharides comprising one or more of the following monomer radicals:
(4) 3-galactopyranose monoradicals, representing 5.31-7.15 mol % of monomer radicals in the plurality of oligosaccharides;
(10) 6-galactopyranose monoradicals, representing 13.81-19.02 mol % of monomer radicals in the plurality of oligosaccharides;
(16) 3,6-galactopyranose and/or 2,6-galactofuranose diradicals, representing 4.14-5.93 mol % of monomer radicals in the plurality of oligosaccharides; and/or
(17) 2,6-galactopyranose diradicals, representing 1.98-2.99 mol % of monomer radicals in the plurality of oligosaccharides.

In some embodiments, the plurality of oligosaccharides comprise at least 2, 3, or 4 of the monomer radicals selected from radicals (4), (10), (16), and (17).

In some embodiments, the oligosaccharide composition further comprises one or more of the following monomer radicals:
(1) t-galactofuranose monoradicals, representing 6.29-12.84 mol % of monomer radicals in the plurality of oligosaccharides;
(2) t-galactopyranose monoradicals, representing 20.45-28.28 mol % of monomer radicals in the plurality of oligosaccharides;
(3) 2-galactofuranose and/or 2-glucofuranose monoradicals, representing 2.73-3.46% mol % of monomer radicals in the plurality of oligosaccharides;
(5) 3-galactofuranose monoradicals, representing 3.36-4.28 mol % of monomer radicals in the plurality of oligosaccharides;
(6) 2-galactopyranose monoradicals, representing 4.12-4.45 mol % of monomer radicals in the plurality of oligosaccharides;
(7) 4-galactopyranose and/or 5-galactofuranose monoradicals, representing 4.65-5.87 mol % of monomer radicals in the plurality of oligosaccharides;
(8) 2,3-galactofuranose diradicals, representing 0.43-0.82 mol % of monomer radicals in the plurality of oligosaccharides;
(9) 6-galactofuranose monoradicals, representing 2.92-9.58 mol % of monomer radicals in the plurality of oligosaccharides;
(11) 3,4-galactopyranose and/or 3,5-galactofuranose and/or 2,3-galactopyranose diradicals, representing 1.41-1.99 mol % of monomer radicals in the plurality of oligosaccharides;
(12) 2,4-glucopyranose and/or 2,5-glucofuranose and/or 2,4-galactopyranose and/or 2,5-galactofuranose diradicals, representing 0.88-1.21 mol % of monomer radicals in the plurality of oligosaccharides;
(13) 2,3,4-galactopyranose and/or 2,3,5-galactofuranose triradicals, representing 0.14-0.28 mol % of monomer radicals in the plurality of oligosaccharides;
(14) 3,6-galactofuranose diradicals, representing 1.69-2.27 mol % of monomer radicals in the plurality of oligosaccharides;
(15) 4,6-galactopyranose and/or 5,6-galactofuranose diradicals, representing 3.93-5.26 mol % of monomer radicals in the plurality of oligosaccharides;
(18) 3,4,6-galactopyranose and/or 3,5,6-galactofuranose and/or 2,3,6-galactofuranose triradicals, representing 0.91-1.68 mol % of monomer radicals in the plurality of oligosaccharides;
(19) 2,3,6-galactopyranose and/or 2,4,6-galactopyranose and/or 2,5,6-galactofuranose triradicals, representing 0.01-3.10 mol % of monomer radicals in the plurality of oligosaccharides; and/or
(20) 2,3,4,6-galactopyranose and/or 2,3,5,6-galactofuranose quadradicals, representing 0.01-0.28 mol % of monomer radicals in the plurality of oligosaccharides.

In some embodiments, an oligosaccharide composition comprises a plurality of oligosaccharides, each oligosaccharide comprising a plurality of monomer radicals;
the plurality of oligosaccharides comprising one or more of the following monomer radicals:
(4) 3-galactopyranose monoradicals, representing 4.79-7.75 mol % of monomer radicals in the plurality of oligosaccharides;
(10) 6-galactopyranose monoradicals, representing 11.64-22.24 mol % of monomer radicals in the plurality of oligosaccharides;
(16) 3,6-galactopyranose and/or 2,6-galactofuranose diradicals, representing 2.20-7.06 mol % of monomer radicals in the plurality of oligosaccharides; and/or
(17) 2,6-galactopyranose diradicals, representing 0.89-3.63 mol % of monomer radicals in the plurality of oligosaccharides.

In some embodiments, the plurality of oligosaccharides comprise at least 2, 3, or 4 of the monomer radicals selected from radicals (4), (10), (16), and (17).

In some embodiments, the oligosaccharide composition further comprises one or more of the following monomer radicals:
(1) t-galactofuranose monoradicals, representing 2.52-15.21 mol % of monomer radicals in the plurality of oligosaccharides;
(2) t-galactopyranose monoradicals, representing 13.49-40.02 mol % of monomer radicals in the plurality of oligosaccharides;
(3) 2-galactofuranose and/or 2-glucofuranose monoradicals, representing 0.64%-4.82 mol % of monomer radicals in the plurality of oligosaccharides;
(5) 3-galactofuranose monoradicals, representing 2.22-5.03 mol % of monomer radicals in the plurality of oligosaccharides;
(6) 2-galactopyranose monoradicals, representing 3.10-5.13 mol % of monomer radicals in the plurality of oligosaccharides;
(7) 4-galactopyranose and/or 5-galactofuranose monoradicals, representing 3.99-6.87 mol % of monomer radicals in the plurality of oligosaccharides;
(8) 2,3-galactofuranose diradicals, representing 0.00-1.93% mol % of monomer radicals in the plurality of oligosaccharides;
(9) 6-galactofuranose monoradicals, representing 1.52-10.39 mol % of monomer radicals in the plurality of oligosaccharides;
(11) 3,4-galactopyranose and/or 3,5-galactofuranose and/or 2,3-galactopyranose diradicals, representing 0.68-3.15 mol % of monomer radicals in the plurality of oligosaccharides;
(12) 2,4-glucopyranose and/or 2,5-glucofuranose and/or 2,4-galactopyranose and/or 2,5-galactofuranose diradicals, representing 0.49-1.45 mol % of monomer radicals in the plurality of oligosaccharides;
(13) 2,3,4-galactopyranose and/or 2,3,5-galactofuranose triradicals, representing 0.00-0.67 mol % of monomer radicals in the plurality of oligosaccharides;
(14) 3,6-galactofuranose diradicals, representing 0.41-3.10 mol % of monomer radicals in the plurality of oligosaccharides;
(15) 4,6-galactopyranose and/or 5,6-galactofuranose diradicals, representing 3.60-5.65 mol % of monomer radicals in the plurality of oligosaccharides;
(18) 3,4,6-galactopyranose and/or 3,5,6-galactofuranose and/or 2,3,6-galactofuranose triradicals, representing 0.68-1.85 mol % of monomer radicals in the plurality of oligosaccharides;
(19) 2,3,6-galactopyranose and/or 2,4,6-galactopyranose and/or 2,5,6-galactofuranose triradicals, representing 0.00-3.51 mol % of monomer radicals in the plurality of oligosaccharides; and/or
(20) 2,3,4,6-galactopyranose and/or 2,3,5,6-galactofuranose quadradicals, representing 0.00-0.35 mol % of monomer radicals in the plurality of oligosaccharides.

In some embodiments, the plurality of oligosaccharides comprise at least 2, 3, 4, 5, 6, 7, 8, 9, 10, 11, 12, 13, 14, 15, or 16 of the monomer radicals selected from radicals (1)-(3), (5)-(9), (11)-(15), and (18)-(20).

In some embodiments, the plurality of oligosaccharides comprise each of the monomer radicals selected from radicals (1)-(20).

In some embodiments, the molar percentages of monomer radicals are determined using a permethylation assay, wherein the permethylation assay comprises gas chromatography-mass spectroscopy (GC-MS) analysis.

In some embodiments, the oligosaccharide composition comprises a plurality of oligosaccharides that consist essentially of Formula (I):

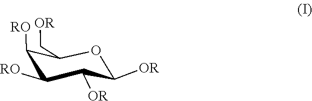

wherein R in Formula (I) is independently selected from hydrogen, and Formulae (Ia), (Ib), (Ic), (Id):

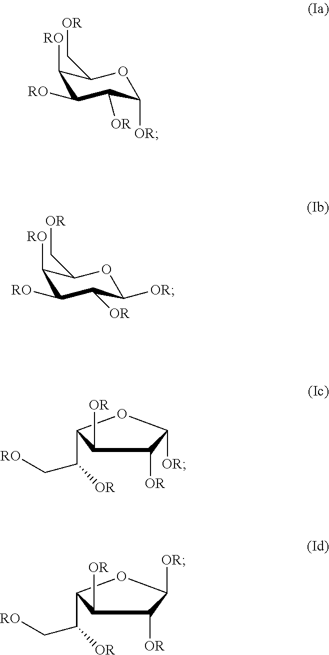

wherein R in Formulae (Ia), (Ib), (Ic), and (Id) is independently defined as above in Formula (I).

In some embodiments, the mean degree of polymerization (DP) of the oligosaccharide composition is from about DP11 to about DP19. In some embodiments, the mean degree of polymerization (DP) of the oligosaccharide composition is from about DP13 to about DP17. In some embodiments, the composition comprises greater than 85% DP2+. In some embodiments, the composition comprises 87-95% DP2+. In some embodiments, the composition comprises 89-93% DP2+. In some embodiments, the composition comprises 58-94% total dietary fiber (dry basis). In some embodiments, the composition comprises 65-87% total dietary fiber (dry basis).

In some embodiments, the oligosaccharide composition comprises a plurality of oligosaccharides that comprise Formula (I):

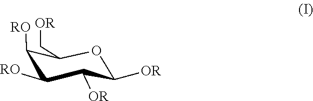

wherein R in Formula (I) is independently selected from hydrogen, and Formulae (Ia), (Ib), (Ic), (Id):

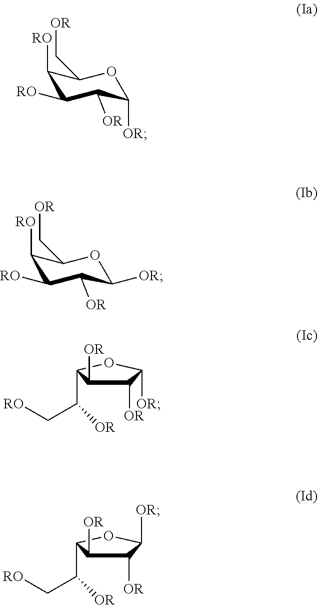

wherein R in Formulae (Ia), (Ib), (Ic), and (Id) is independently defined as above in Formula (I);
wherein the oligosaccharide composition is produced by a process comprising:
(a) forming a reaction mixture comprising galactose monomer with an acid catalyst comprising positively charged hydrogen ions; and
(b) promoting acid catalyzed oligosaccharide formation in the reaction mixture by transferring sufficient heat to the reaction mixture to maintain the reaction mixture at its boiling point.

In some embodiments, step (b) comprises loading the reaction mixture with an acid catalyst comprising positively charged hydrogen ions, in an amount such that the molar ratio of positively charged hydrogen ions to total galactose monomer content is in an appropriate range. In some embodiments, steps (a) and (b) occur simultaneously. In some embodiments, step (a) comprises heating the reaction mixture under agitation conditions to a temperature in a range of 100° C. to 160° C. In some embodiments, step (a) comprises heating the reaction mixture under agitation conditions to a temperature in a range of 130° C. to 140° C. In some embodiments, step (a) comprises gradually increasing the temperature (e.g., from room temperature) to about 136° C., under suitable conditions to achieve homogeneity and uniform heat transfer.

In some embodiments, step (b) comprises maintaining the reaction mixture at atmospheric pressure or under vacuum, at a temperature in a range of 128° C. to 140° C. (optionally 130° C. to 140° C.), under conditions that promote acid catalyzed oligosaccharide composition formation, until the weight percent of galactose monomer in the oligosaccharide composition is in a range of 5-14%. In some embodiments, step (b) comprises maintaining the reaction mixture at atmospheric pressure or under vacuum, at a temperature in a range of 128° C. to 140° C. (optionally 130° C. to 140° C.), under conditions that promote acid catalyzed oligosaccharide composition formation, until the weight percent of galactose monomer in the oligosaccharide composition is in a range of 5-13%. In some embodiments, step (b) comprises maintaining the reaction mixture at atmospheric pressure or under vacuum, at a temperature in a range of 128° C. to 140° C. (optionally 130° C. to 140° C.), under conditions that promote acid catalyzed oligosaccharide composition formation, until the weight percent of galactose monomer in the oligosaccharide composition is in a range of 7-11%. In some embodiments, step (b) comprises maintaining the reaction mixture at atmospheric pressure or under vacuum, at a temperature of about 136° C., under conditions that promote acid catalyzed oligosaccharide composition formation, until the weight percent of galactose monomer in the oligosaccharide composition is in a range of 5-13%. In some embodiments, step (b) comprises maintaining the reaction mixture at atmospheric pressure or under vacuum, at a temperature of about 136° C., under conditions that promote acid catalyzed oligosaccharide composition formation, until the weight percent of galactose monomer in the oligosaccharide composition is in a range of 7-11%.

In some embodiments, the acid catalyst is a strong acid cation exchange resin having one or more physical and chemical properties according to Table 1 and/or wherein the catalyst comprises >3.0 mmol/g sulfonic acid moieties and <1.0 mmol/gram cationic moieties. In some embodiments, the catalyst has a nominal moisture content of 45-50 weight percent. In some embodiments, the acid catalyst is a soluble catalyst. In some embodiments, the soluble catalyst is an organic acid. In some embodiments, the soluble catalyst is a weak organic acid. In some embodiments, the soluble catalyst is citric acid.

In some embodiments, the process further comprises (c) quenching the reaction mixture, for example, using water, while bringing the temperature of the reaction mixture to a temperature in the range of 55° C. to 95° C. (e.g., 85° C., 90° C.). In some embodiments, the process (e.g., a large-scale process, e.g., a 50 L, 2000 L, or greater than 50 L process) further comprises (c) quenching the reaction mixture, for example, using water, while bringing the temperature of the reaction mixture to a temperature in the range of 20° C. to 40° C. (e.g., 20° C., 25° C.).

In some embodiments, the process further comprises (d) separating oligosaccharide composition from the acid catalyst. In some embodiments, said separating comprises removing the catalyst by filtration. In some embodiments, (d) comprises cooling the reaction mixture to below about 100° C. before filtering.

In some embodiments, the process further comprises: (e) diluting the oligosaccharide composition of (d) with water to a concentration of about 40-55 weight percent (optionally 45-55 weight percent); (f) passing the diluted composition through a cationic exchange resin; (g) passing the diluted composition through a decolorizing polymer resin; and/or (h) passing the diluted composition through an anionic exchange resin; wherein each of (f), (g), and (h) can be performed one or more times in any order.

In some aspects, provided herein is a method of reducing inflammation in a subject. In some embodiments, a method of reducing inflammation in a subject comprises administering to the gastrointestinal tract of the subject an effective amount of an oligosaccharide composition described herein.

In some aspects, provided herein is a method of treating a subject having or suspected of having an inflammatory and immune disorder. In some embodiments, a method of treating a subject having or suspected of having an inflammatory and immune disorder comprises administering to the gastrointestinal tract of the subject an effective amount of an oligosaccharide composition described herein, thereby treating the subject.

In some aspects, provided herein is a method of treating a subject having or suspected of having an inflammatory and immune disorder, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I):

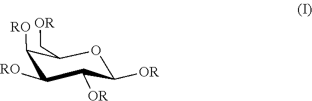

wherein R in Formula (I) is independently selected from hydrogen, and Formulae (Ia), (Ib), (Ic), (Id):

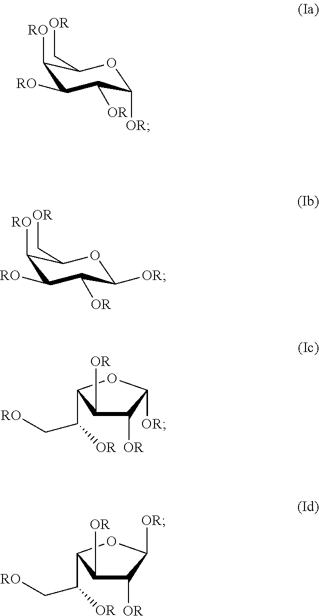

wherein R in Formulae (Ia), (Ib), (Ic), and (Id) is independently defined as above in Formula (I), thereby treating the subject.

In some aspects, provided herein is a method of treating a subject having or suspected of having an inflammatory bowel disease. In some embodiments, a method of treating a subject having or suspected of having an inflammatory bowel disease comprises administering to the gastrointestinal tract of the subject an effective amount of an oligosaccharide composition described herein, thereby treating the subject.

In some aspects, provided herein is a method of treating a subject having or suspected of having an inflammatory bowel disease comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I):

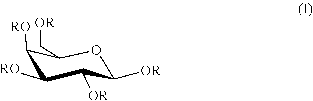

wherein R in Formula (I) is independently selected from hydrogen, and Formulae (Ia), (Ib), (Ic), (Id):

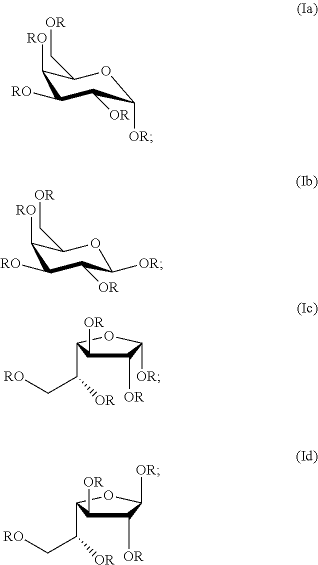

wherein R in Formulae (Ia), (Ib), (Ic), and (Id) is independently defined as above in Formula (I), thereby treating the subject.

In some embodiments, the inflammatory and immune disorder is a chronic inflammatory disorder. In some embodiments, the chronic inflammatory disorder is inflammatory bowel disease.

In some embodiments, the inflammatory bowel disease is ulcerative colitis. In some embodiments, the inflammatory bowel disease is Crohn's disease. In some embodiments, the inflammatory bowel disease is granulomatous colitis. In some embodiments, the inflammatory bowel disease is indeterminate colitis. In some embodiments, the inflammatory bowel disease is diversion colitis. In some embodiments, the inflammatory bowel disease is pouchitis. In some embodiments, the inflammatory bowel disease is Behcet's disease. In some embodiments, the inflammatory bowel disease is microscopic colitis. In some embodiments, the inflammatory bowel disease is diverticulosis-associated colitis. In some embodiments, the inflammatory bowel disease is collagenous colitis. In some embodiments, the inflammatory bowel disease is lymphocytic colitis. In some embodiments, the inflammatory bowel disease is pediatric-onset inflammatory bowel disease.

In some aspects, provided herein is a method of increasing the relative or absolute abundance of short chain fatty acids in a subject. In some embodiments, a method of increasing the relative or absolute abundance of short chain fatty acids in a subject comprises administering to the gastrointestinal tract of the subject an effective amount of an oligosaccharide composition described herein.

In some embodiments, the relative or absolute abundance of short chain fatty acids is increased by at least 5%, 10%, 20%, or 30%, compared to a baseline measurement (e.g., wherein the baseline measurement is determined prior to treatment),

In some embodiments, the short chain fatty acids are butyrate, acetate, and/or propionate.

In some aspects, provided herein is a method of decreasing the relative or absolute abundance of pro-inflammatory and/or pathogenic bacteria in a subject. In some embodiments, a method of decreasing the relative or absolute abundance of pro-inflammatory and/or pathogenic bacteria in a subject comprises administering to the gastrointestinal tract of the subject an effective amount of an oligosaccharide composition described herein. In some embodiments, the pro-inflammatory and/or pathogenic bacteria are Enterobacteriaceae and/or Ruminococcaceae.

In some aspects, provided herein is a method of increasing the relative or absolute abundance of commensal bacteria in a subject. In some embodiments, a method of increasing the relative or absolute abundance of commensal bacteria in a subject comprises administering to the gastrointestinal tract of the subject an effective amount of an oligosaccharide composition described herein. In some embodiments, the commensal bacteria are Parabacteroides and/or Bacteroides.

In some embodiments, the subject is a human subject. In some embodiments, the subject is a newborn (a preterm newborn, a full-term newborn), an infant up to one year of age, a young child (e.g., 1 year to 12 years), a teenager (e.g., 13-19 years), an adult (e.g., 20-64 years), or an elderly adult (e.g., 65 years and older).

In some embodiments, the method comprises administering the oligosaccharide composition to the intestines (e.g., the large intestine). In some embodiments, the oligosaccharide composition is self-administered to the subject. In some embodiments, the oligosaccharide composition is formulated as a pharmaceutical composition for oral delivery. In some embodiments, the oligosaccharide composition is orally administered to the subject. In some embodiments, the oligosaccharide composition is administered to the subject once per day or twice per day.

In some embodiments, the method increases the abundance or concentration of total short chain fatty acids in the subject (e.g., the gastrointestinal tract of the subject).

In some embodiments, the method increases the abundance or concentration of butyrate in the subject (e.g., the gastrointestinal tract of the subject). In some embodiments, the method increases the abundance or concentration of propionate in the subject (e.g., the gastrointestinal tract of the subject). In some embodiments, the method increases the abundance or concentration of acetate in the subject (e.g., the gastrointestinal tract of the subject).

In some embodiments, the abundance of total SCFAs are increased by at least 5%, 10%, 20%, or 30%, relative to a baseline measurement (e.g., wherein the baseline measurement is determined prior to treatment). In some embodiments, the abundance of at least one of butyrate, propionate, and acetate are increased by at least 5%, 10%, 20%, or 30%, relative to a baseline measurement (e.g., wherein the baseline measurement is determined prior to treatment).

In some embodiments, the method promotes the growth of commensal bacteria within the microbiome of the gastrointestinal tract of the subject (e.g., increases their relative abundance). In some embodiments, the method promotes the growth of Parabacteroides and Bacteroides within the microbiome of the gastrointestinal tract of the subject (e.g., increases their relative abundance).

In some embodiments, the method causes a decrease in the abundance of pro-inflammatory and/or pathogenic bacteria within the microbiome of the gastrointestinal tract of the subject. In some embodiments, the method causes a decrease in the abundance of pro-inflammatory Enterobacteriaceae within the microbiome of the gastrointestinal tract of the subject.

In some embodiments, the method results in a decrease in the levels of fecal calprotectin, fecal lipocalin, and/or fecal lactoferrin in a stool/fecal sample belonging to the subject, relative to a baseline measurement. In some embodiments, the level of fecal calprotectin is decreased by at least 50%, relative to a baseline measurement. In some embodiments, the level of fecal calprotectin is decreased by at least 65%, relative to a baseline measurement. In some embodiments, the level of fecal lactoferrin is decreased by at least 50%, relative to a baseline measurement.

In some embodiments, the method causes a depletion of genes associated with adherent-invasive E. coli within the microbiome of the gastrointestinal tract of the subject. In some embodiments, the genes associated with adherent-invasive E. coli are fimH, ompA, and ompC.

In some embodiments, the oligosaccharide composition is administered for at least 20, 30, 40, or 50 days. In some embodiments, the oligosaccharide composition is administered for 56 days or 10 weeks. In some embodiments, the oligosaccharide composition is administered for 20-100 days, optionally 50-75 days.

In some embodiments, the subject has ulcerative colitis, and wherein the administration of the oligosaccharide composition results in a decrease in ulcerative colitis disease activity, relative to a baseline measurement. In some embodiments, the decrease in ulcerative colitis disease activity is measured using the Simple Clinical Colitis Activity Index (SCCAI) composite score.

In some embodiments, the method further comprises administering a standard-of-care treatment. In some embodiments, the standard-of-care treatment is 5-ASA (mesalamine), azathioprine, Vedolizumab, Infliximab, or Adalimumab.

Some aspects provide a method of decreasing the levels of one or more biomarkers associated with inflammation (e.g., fecal calprotectin, fecal lipocalin, and/or fecal lactoferrin) in a subject, optionally a subject exhibiting an inflammatory disease, comprising administering the oligosaccharide composition of any one of claims 1-57 to the subject in an effective amount to decrease levels of the one or more biomarkers, relative to a baseline measurement.

Some aspects provide a method of decreasing the abundance of one or more pathobionts (e.g., pro-inflammatory bacterial taxa such as Enterobacteriaceae) in a subject, optionally a subject exhibiting an inflammatory disease, comprising administering the oligosaccharide composition of any one of claims 1-57 to the subject in an effective amount to decrease the abundance of the one or more pathobionts.

Some aspects provide a method of increasing the abundance of one or more commensal taxa (e.g., Parabacteroides and Bacteroides) in a subject, optionally a subject exhibiting an inflammatory disease, comprising administering the oligosaccharide composition of any one of claims 1-57 to the subject in an effective amount to increase the abundance of the one or more commensal taxa.

In some embodiments, levels of the one or more biomarkers, abundance of the one or more pathobionts, and/or abundance of the one or more commensal taxa is measured in fecal/stool samples from the subject.

### BRIEF DESCRIPTION OF THE DRAWINGS

FIG. 1 provides a graph showing the ability of the selected oligosaccharide composition to produce an increase in the concentration of butyrate in fecal samples from eight healthy human subjects relative to a negative control (water).

FIGS. 2A-2B provide graphs showing the ability of the selected oligosaccharide composition to produce an increase in the concentration of short chain fatty acids (butyrate, propionate, and acetate) in fecal samples from eight healthy human subjects relative to a negative control (water). FIG. 2A shows the median amount of short chain fatty acids (in mM) produced across the tested fecal samples. FIG. 2B shows the relative proportions of butyrate, propionate, and acetate produced in each tested fecal sample.

FIGS. 3A-3B provide graphs showing the ability of the selected oligosaccharide composition to modulate the abundance of pathogenic and commensal bacteria in fecal samples from eight healthy human subjects relative to a negative control (water). FIG. 3A shows that the selected oligosaccharide composition causes a decrease in the relative abundance of a pathogenic genus (Enterobacteriaceae). FIG. 3B shows that the selected oligosaccharide composition causes an increase in the relative abundance of commensal genera (Parabacteroides and Bacteroides).

FIG. 4 provides a schematic design for a clinical food study trial to assess safety and tolerability of the selected oligosaccharide composition in in subjects with ulcerative colitis (UC), as well as microbiome changes and changes in inflammatory biomarkers modulated by the selected oligosaccharide composition.

FIG. 5 provides an example HSQC NMR pulse sequence diagram.

FIGS. 6A-6C provide HSQC NMR data relating to the selected oligosaccharide composition. FIG. 6A shows an HSQC NMR spectrum of the selected oligosaccharide obtained using the methods described in Example 8 (F2 dimension=¹H; F1 dimension=¹³C). FIG. 6B shows the integral regions that define the coordinates of HSQC NMR peaks/signals 1-11 of the selected oligosaccharide composition using an elliptical shape (F2 dimension=¹H; F1 dimension=¹³C). FIG. 6C shows an example of an elliptical shape defined by major axis coordinates (F2 dimension; ¹H) and minor axis coordinates (F1 dimension; ¹³C).

FIGS. 7A-7B provide images of an HSQC NMR spectrum of the selected oligosaccharide obtained using the methods described in Example 8 (F2 dimension=¹H; F1 dimension=¹³C). FIG. 7A shows an expanded view of the non-anomeric region. Annotations indicate assigned locations of discrete bond types present within the selected oligosaccharide; and peaks/signals 7-11 of the selected oligosaccharide composition. FIG. 7B shows an expanded view of the anomeric region. Annotations indicate assigned locations of discrete bond types present within the selected oligosaccharide; and peaks/signals 1-6 of the selected oligosaccharide composition.

FIG. 8 provides graphs demonstrating the effect of holding the selected oligosaccharide composition at various temperatures over a period of six hours in the presence of Water.

FIG. 9 provides example mechanisms through which the selected oligosaccharide composition is thought to reduce intestinal inflammation.

FIG. 10 provides graphs showing the ability of the selected oligosaccharide composition to increase the concentration of total short-chain fatty acids (SCFA) and individual SCFAs (acetate, propionate, and butyrate) in fecal samples from ten healthy human subjects compared to a negative control (water).

FIG. 11 provides a Bray-Curtis non-metric multi-dimensional scaling (NMDS) ordination plot showing that the selected oligosaccharide composition shifts the composition of microbiomes in fecal samples from ten healthy human subjects compared to a negative control (water). Each data point in the plot represents the microbiome composition from an individual fecal sample. The circled data points represent samples incubated with the selected oligosaccharide composition.

FIG. 12 provides a heatmap showing the log 2-fold change in relative abundance of bacterial taxa (columns) in ten fecal samples (rows) from healthy subjects after incubation with the selected oligosaccharide composition compared to a negative control (water). Depleted taxa (including pathobionts) across the samples are indicated and on left side of the heatmap; enriched taxa (including commensals) are indicated and in center and right side of the heatmap.

FIGS. 13A-13B provide graphs showing the ability of the selected oligosaccharide composition to modulate the abundance of pathobiont and commensal bacteria in fecal samples from eight healthy human subjects relative to a negative control (water). FIG. 13A shows that the effect of the selected oligosaccharide composition on the relative abundance of a commensal genus (Parabacteroides). FIG. 13B shows that the effect of the selected oligosaccharide composition on the relative abundance of a pathobiont genera (Enterobacteriaceae).

FIGS. 14A-14B provide graphs showing the effect of the selected oligosaccharide composition on the growth of commensal bacterial taxa (FIG. 14A) (Parabacteroides merdae, Parabacteroides distansonis, Bacteroides uniformis, Bacteroides thetaiotaomicron, and Bacteroides caccae) and pathobiont taxa (FIG. 14B) (Escherichia coli, Klebsiella pneumoniae, Enterobacter cloacae, and Salmonella enterica) in single-strain assays, with negative control (water, to assess minimum growth without an added carbon source) and positive control (glucose, as universal carbon source to assess maximum growth capacity of the assay).

FIGS. 15A-15B provide graphs showing the ability of the selected oligosaccharide composition to decrease the levels of fecal biomarkers in human patients having ulcerative colitis (UC) following participation in the clinical trial described in Example 11. FIG. 15A shows levels of fecal calprotectin in fecal samples of patients at screening and at the end of intake (see, FIG. 4) plotted as a data set (left) and for each individual (right). FIG. 15B shows levels of fecal lactoferrin in fecal samples of patients before and at the end of intake (see, FIG. 4) plotted as a data set (left) and for each individual (right).

FIG. 16 provides a Simple Clinical Colitis Activity Index (SCCAI) composite score for patients with ulcerative colitis (UC) following administration of the selected oligosaccharide composition in the clinical trial described in Example 11, before and at the end of intake (see, FIG. 4) plotted as a data set (left) and for each individual (right).

FIGS. 17A-17B provide graphs showing the abundance of selected pathogenic and commensal bacterial taxa in fecal samples from five patients with ulcerative colitis (UC) following participation in the clinical trial described in Example 11, before and at the end of intake (see, FIG. 4) plotted as a data set (left) and for each individual (right). FIG. 17A shows that the relative abundance of Parabacteroides taxa. FIG. 15B shows that the relative abundance of Enterobacteriaceae taxa.

FIG. 18 provides graphs showing changes of three genes associated with adherent-invasive E. coli (fimH, ompA, and ompC) in fecal samples of patients with ulcerative colitis (UC) following participation in the clinical trial described in Example 11, before and at the end of intake (see, FIG. 4) plotted as a data set (left) and for each individual (right).

FIG. 19 provide graphs showing the screening effort that was performed to identify the selected oligosaccharide composition, indicating mean butyrate production across 431 synthetic oligosaccharides that were screened and that of the selected oligosaccharide.

FIG. 20 provides a schematic design for a clinical study to assess the ability of the selected oligosaccharide composition to treat patients having mild to moderately active ulcerative colitis.

### DETAILED DESCRIPTION OF THE INVENTION

The compositions and methods described herein are based on the discovery that oligosaccharide compositions are useful for reducing inflammation in a subject. In some embodiments, the oligosaccharide compositions described herein are useful in producing increased levels of short chain fatty acids (SCFAs) such as butyrate, propionate, and acetate in a subject. In some embodiments, the oligosaccharide compositions described herein are useful for decreasing the abundance of pro-inflammatory microbial taxa (e.g., taxa from the Enterobacteriaceae family) relative to commensal microbes (e.g., Parabacteroides and Bacteroides) in a subject (e.g., the gastrointestinal tract of a subject). In some embodiments, inflammation is reduced in a subject due to increased levels of SCFAs in the subject and/or decreased relative abundance of pro-inflammatory microbes in the subject (e.g., the gastrointestinal tract of a subject). In some embodiments, the oligosaccharide compositions described herein are useful for treating inflammatory and immune disorders including autoimmune and allergic disorders. In some embodiments, the oligosaccharide compositions described herein are useful in treating chronic inflammatory disorders, e.g., inflammatory bowel diseases. In some embodiments, the oligosaccharide compositions described herein are useful in treating inflammatory bowel diseases such as ulcerative colitis (UC), Crohn's disease (CD), granulomatous colitis, indeterminate colitis, diversion colitis, pouchitis, Behcet's Disease, microscopic colitis, diverticulosis-associated colitis, collagenous colitis, lymphocytic colitis, and pediatric-onset inflammatory bowel disease. In some embodiments, the oligosaccharide compositions described herein are useful in treating inflammatory bowel diseases such as ulcerative colitis (UC).

Some aspects of the disclosure are based on the results of an extensive screening effort that was performed to identify oligosaccharide compositions that are capable of modulating, e.g., increasing, the concentration of different types of short chain fatty acids, e.g., butyrate, propionate, and acetate, in a subject. Hundreds of unique oligosaccharide compositions were assayed for their effect on the microbiome of human gastrointestinal tracts in an ex vivo context. The oligosaccharide compositions examined in the screen were produced using different saccharide monomers, e.g., dextrose monomers, xylose monomers, etc., and under conditions involving differing reaction temperatures, for varying periods of time, and/or in the presence of different catalyst conditions. From this screening effort, a selected oligosaccharide composition was identified as a highly effective modulator of SCFAs, e.g., butyrate, propionate, and acetate.

SCFAs such as butyrate, propionate, and acetate serve important roles in the maintenance of healthy epithelial function, immune homeostasis, and inflammation in the gastrointestinal tract of a subject. Additionally, SCFAs stimulate the gut epithelium to exert colonization resistance on pathogenic bacteria and promote the differentiation and function of regulatory T cells in the gastrointestinal tract. Butyrate is used as a source of energy for epithelial cells of the gastrointestinal tract and promotes maintenance of epithelial integrity. Maintenance of epithelial integrity is important to prevent inappropriate activation of innate and adaptive immune cells. Butyrate activates G-protein coupled receptors (GPCRs) displayed on the surface of epithelial and immune cells, as well as inhibits histone deacetylases in the nucleus of these cell types. Engagement of these host cell targets promotes the proper balance of immune functions and immune homeostasis in the gastrointestinal tract. Propionate can also promote gut immune homeostasis by activating these same GPCRs. Increased levels of these SCFAs in the gastrointestinal tract of a subject corresponds to a reduction in inflammation and/or a reduced likelihood of inflammation in the gastrointestinal tract. Accordingly, administration of the selected oligosaccharide composition described herein to a subject, which causes an increase in SCFA levels in said subject, ultimately leads to a reduction in inflammation and/or a reduced likelihood of inflammation in the gastrointestinal tract.

In addition to producing increased levels of SCFAs, fermentation of the selected oligosaccharide composition results in the growth of the commensal bacteria (e.g., Parabacteroides and Bacteroides) and creates a nutritionally competitive ecosystem in the gastrointestinal tract. This competitive environment helps prevent, slow, or limit colonization by pro-inflammatory, pathogenic bacteria (e.g., taxa from the Enterobacteriaceae family) that can disrupt gut homeostasis and stimulate harmful inflammatory immune response. The shift in the microbial ecosystem of the gastrointestinal tract to reduce the relative abundance of pro-inflammatory microbes, resulting from administration of the selected oligosaccharide composition, causes a reduction in inflammation and/or a reduced likelihood of inflammation in the gastrointestinal tract.

Accordingly, in some embodiments, this oligosaccharide composition is particularly useful for treating subjects having dysbiosis, high relative abundance of pathogenic bacteria relative to commensal bacteria, and/or low levels of SCFAs. In some embodiments, the selected oligosaccharide composition is useful to treat inflammatory and immune disorders. In some embodiments, the selected oligosaccharide composition is useful to treat autoimmune and allergic disorders. In some embodiments, the selected oligosaccharide composition is useful to treat chronic inflammatory disorders. In some embodiments, the selected oligosaccharide composition is useful to treat inflammatory bowel diseases (e.g., ulcerative colitis (UC), Crohn's disease (CD), granulomatous colitis, indeterminate colitis, diversion colitis, pouchitis, Behcet's disease, microscopic colitis, diverticulosis-associated colitis, collagenous colitis, lymphocytic colitis, and pediatric-onset inflammatory bowel disease). A hallmark of inflammatory and immune disorders, particularly chronic inflammatory disorders affecting the gastrointestinal tract (e.g., inflammatory bowel diseases), is a profound alteration in the composition of the gut microbiota, dysbiosis, with reproducible findings across multiple clinical studies showing an increase in the presence of pro-inflammatory taxa (e.g., taxa from the Enterobacteriaceae family) and a decrease in commensal diversity associated with normal immune activity. Thus, increased production of short chain fatty acids and/or an increase in abundance of commensal bacteria relative to pro-inflammatory bacteria in the gastrointestinal tract of a subject (e.g., resulting from administration of the selected oligosaccharide composition as described herein to the gastrointestinal tract of the subject) can lead to treatment of inflammatory and immune disorders such as chronic inflammatory disorders (e.g., inflammatory bowel diseases, e.g., ulcerative colitis or Crohn's disease).

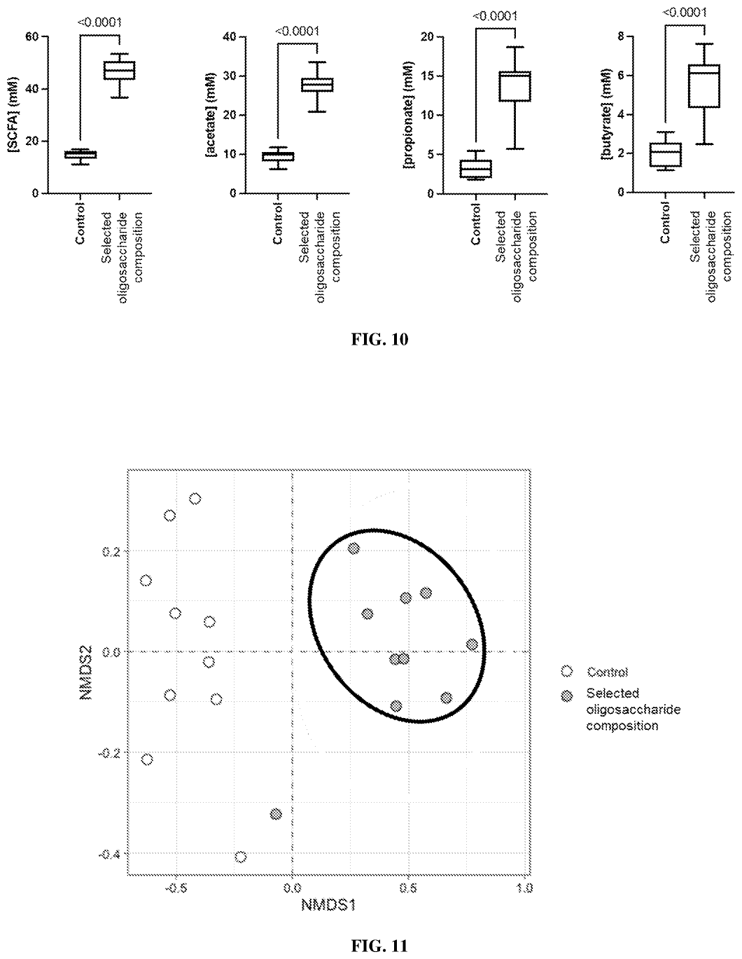

In some embodiments, the selected oligosaccharide composition functions to reduce inflammation, e.g., chronic digestive tract and/or systemic inflammation (e.g., inflammation associated with ulcerative colitis) by (a) stimulating growth, metabolism, and/or nutrient utilization by beneficial bacteria, to indirectly limit pathogenic growth via nutrient competition; and/or promoting the production of SCFAs and other microbial metabolites, which support intestinal epithelial function in reducing inflammation. In some embodiments, the selected oligosaccharide composition is thought to reduce inflammation using one or more, or all of the mechanisms described by FIG. 9, wherein, for example, the oligosaccharide composition preferentially supports the growth of beneficial taxa (e.g., commensal taxa) that produce SCFAs and other useful metabolities) and does not support the growth of (bacterial) pathobionts or pathogens. SCFAs and other metabolites support the intestinal epithelia and modulate gut inflammation, which in turn, can lead to an improvement in barrier function. These changes in host epithelium and gut microbiota can directly lower GI tract inflammation in UC subjects; and further limit systemic inflammation.

In some embodiments, the selected oligosaccharide composition is useful to treat inflammatory bowel diseases such as ulcerative colitis (UC). Ulcerative colitis causes recurrent inflammation and ulcers in the mucosa of the large intestine and rectum. Although the exact pathophysiology of UC is not known, multiple lines of evidence suggest that the disease is caused by a confluence of genetic and environmental factors that alter gut homeostasis, thereby triggering immune-mediated inflammation. UC comprises three groupings of disease activity and progression—mild activity (modified Mayo score of 2-4), moderate activity (moderate Mayo score of 5-7), and severe activity (modified Mayo score of 8-9). Subjects having mild UC activity generally have less than or equal to 4 stools/day with or without blood and limited erythema in superficial mucosa, and are typically biologics-naïve, with long-term 5-ASAs to promote remission and flares commonly managed by short-course steroids. Subjects having moderate UC activity generally have 5 or more stools/day and mild, but increasing anemia due to diarrhea and moderate ulceration of colon in endoscopy. A typical subject having moderate UC activity progresses from mild to moderate after 5-ASA treatment and typically treated with IS/steroids and early-line biologics (e.g., anti-TNFs, vedolizumab). Subjects having severe UC activity generally have 8 or more stools/day of profuse bloody diarrhea, with endoscopy revealing loss of mucosal vascular markings. Patients that progress from moderate to severe are primarily treated with biologics and small molecules, with colectomy considered as the last-line option.

The majority of UC patients are diagnosed at 30 to 40 years of age with a slightly elevated prevalence among males (men comprise ˜60% of UC patients) and experience their worst symptoms between their 30 s to 50 s. As patients age, their UC disease activity tends to decline. Genetic predisposition is considered a key risk factor for developing UC, but it has been suggested that environmental triggers might be necessary for onset of inflammation. UC patients often experience co-morbidities in the form of autoimmune extraintestinal manifestations (e.g., rheumatoid arthritis, primary sclerosing cholangitis), and may thus require additional medical or pharmacological intervention for other autoimmune conditions, in addition to their UC treatments.

UC can be diagnosed using microbial fecal testing, endoscopy, biomarker analysis, and routine blood panels. Initial microbial fecal tests can be used to determine infectious causes of colon inflammation (e.g., Salmonella, C. Difficile, Campylobacter infections) via PCR while microscopic fecal analysis may also be conducted for common intestinal parasites. A UC diagnosis can be confirmed by endoscopy, e.g., to identify continuous ulcerations across colonic mucosa (while “skip areas” of diseased tissue supports a Crohn's diagnosis). Biomarker analysis for UC diagnosis comprises, in some embodiments, yearly testing of C-reactive protein (CRP) levels to monitor remission.

In some embodiments, the selected oligosaccharide composition is useful to treat inflammatory bowel diseases such as Crohn's disease (CD). Crohn's disease causes inflammation of the gastrointestinal tract (particularly in last section of the small intestine and colon), leading to abdominal pain, severe diarrhea, fatigue, weight loss and malnutrition.

In some embodiments, the selected oligosaccharide composition is useful to treat inflammatory bowel diseases such as granulomatous colitis. Granulomatous colitis causes inflammation of the gastrointestinal tract, mural thickening and a loss of mural stratification. In some embodiments, granulomatous colitis is most prevalent in the terminal ileum.

In some embodiments, the selected oligosaccharide composition is useful to treat inflammatory bowel diseases such as indeterminate colitis. Indeterminate colitis is characterized by inflammation of the gastrointestinal tract and typical symptoms of inflammatory bowel diseases. In some embodiments, a subject is diagnosed with indeterminate colitis because the histology results are inconclusive for any of the other inflammatory bowel diseases (e.g., UC or CD).

In some embodiments, the selected oligosaccharide composition is useful to treat inflammatory bowel diseases such as diversion colitis. Diversion colitis is an inflammation of the colon resulting from complications of surgical procedures (e.g., ileostomy or colostomy), often occurring within the year after the procedure.

In some embodiments, the selected oligosaccharide composition is useful to treat inflammatory bowel diseases such as pouchitis. Pouchitis is an inflammation in the lining of a pouch created during a surgical procedure (e.g., J pouch surgery) to treat other inflammatory bowel diseases such as ulcerative colitis or other diseases of the gastrointestinal tracts, certain other diseases. Approximately 25-50% of patients who have J pouch surgery experience pouchitis.

In some embodiments, the selected oligosaccharide composition is useful to treat inflammatory bowel diseases such as Behcet's disease. Behcet's disease is a rare disorder that causes blood vessel inflammation throughout the body. In some embodiments, the gastrointestinal tract experiences moderate-to-severe inflammation, abdominal pain, diarrhea, and bleeding.

In some embodiments, the selected oligosaccharide composition is useful to treat inflammatory bowel diseases such as microscopic colitis. Microscopic colitis causes an inflammation of the large intestine that causes persistent watery diarrhea. In some embodiments, microscopic colitis is further classified as collagenous colitis (characterized by a thick layer of collagen in colon tissue), lymphocytic colitis (characterized by increased lymphocytes in colon tissue), or incomplete microscopic colitis (characterized by combination of features of collagenous and lymphocytic colitis).

In some embodiments, the selected oligosaccharide composition is useful to treat inflammatory bowel diseases such as diverticulosis-associated colitis. Diverticulosis-associated colitis is characterized by chronic inflammation in the sigmoid colon affected by diverticular disease. Inflammation may be located in the luminal mucosa.

In some embodiments, the selected oligosaccharide composition is useful to treat inflammatory bowel diseases such as pediatric-onset inflammatory bowel disease. Pediatric-onset inflammatory bowel disease, which typically causes inflammation in the colon and can be resistant to standard-of-care medications, affects persons under 17 years of age. In some embodiments, pediatric-onset inflammatory bowel disease is further classified as early-onset inflammatory bowel disease (characterized by disease onset under 10 years of age), very early-onset inflammatory bowel disease (characterized by disease onset under 6 years of age), infantile inflammatory bowel disease (characterized by disease onset under 2 years of age), or neonatal inflammatory bowel disease (characterized by disease onset under 28 days of age).

Further aspects of the disclosure, including a description of defined terms, are provided below.

### I. Definitions

Agitation conditions: As used herein, the term “agitation conditions” refers to conditions that promote or maintain a substantially uniform or homogeneous state of a mixture (e.g., a reaction mixture comprising galactose monomer) with respect to dispersal of solids (e.g., solid catalysts), uniformity of heat transfer, or other similar parameters. Agitation conditions generally include stirring, shaking, and/or mixing of a reaction mixture. In some embodiments, agitation conditions may include the addition of gases or other liquids into a solution. In some embodiments, agitation conditions are used to maintain substantially uniform or homogenous distribution of a catalyst, e.g., an acid catalyst. In some embodiments, a monosaccharide preparation is heated in the presence of an acid catalyst under suitable conditions to achieve homogeneity and uniform heat transfer in order to synthesize an oligosaccharide composition.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Effective amount: As used herein, the term “effective amount” refers to an administered amount or concentration of an oligosaccharide composition that is necessary and sufficient to elicit a biological response, e.g., in a subject or patient. In some embodiments, an effective amount of an oligosaccharide composition is capable of increasing the concentration of short-chain fatty acids (e.g., butyrate, propionate, and/or acetate) in a subject (e.g., in the gastrointestinal tract of a subject). In some embodiments, an effective amount of an oligosaccharide composition is capable of modulating, e.g., increasing or decreasing, the concentration or number of at least one microbial species. In some embodiments, an effective amount of an oligosaccharide composition is capable of reducing the acquisition of, colonization of, or reducing the reservoir of a pro-inflammatory microbe and/or pathogen (e.g., a drug or antibiotic resistant pathogen, or an MDR pathogen) in a subject. In some embodiments, an effective amount of an oligosaccharide composition is capable of decreasing the abundance of pro-inflammatory and/or pathogenic microbes relative to commensal microbes. In some embodiments, an effective amount of an oligosaccharide composition is capable of treating a subject having autoimmune and allergic disorders (e.g., chronic inflammatory disorders such as inflammatory bowel diseases). In some embodiments, an effective amount of an oligosaccharide composition is capable of treating ulcerative colitis (UC), Crohn's disease (CD), granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease. In some embodiments, an effective amount of an oligosaccharide composition is capable of modulating, e.g., decreasing, the symptoms of autoimmune and allergic disorders (e.g., chronic inflammatory disorders such as inflammatory bowel diseases) in a subject (e.g., the severity or number of symptoms). In some embodiments, an effective amount of an oligosaccharide composition is capable of modulating, e.g., increasing or decreasing, the activity or levels of an enzyme in a subject. In some embodiments, an effective amount of an oligosaccharide composition is capable of modulating, e.g., increasing or decreasing, the processing of a metabolite.

Galactose monomer: As used herein, the term “galactose monomer” generally refers to a D-isomer of a galactose monomer, known as _{D}-galactose.

Monosaccharide Preparation: As used herein, the term “monosaccharide preparation” refers to a preparation that comprises galactose monomer. In some embodiments, a monosaccharide preparation comprises galactose monomers.

Oligosaccharide: As used herein, the term “oligosaccharide” (which may be used interchangeably with the term “oligosaccharide” in some contexts) refers to a saccharide molecule comprising galactose monomers linked together via a glycosidic bond (having a degree of polymerization (DP) of at least 2 (e.g., DP2+)). In some embodiments, an oligosaccharide comprises at least two, at least three, at least four, at least five, at least six, at least seven, at least eight, at least nine, or at least ten monosaccharides subunits linked by glycosidic bonds. In some embodiments, an oligosaccharide is in the range of 3-20, 4-16, 5-15, 8-12, 5-25, 10-25, 20-50, 40-80, or 75-100 monosaccharides linked by glycosidic bonds. In some embodiments, an oligosaccharide comprises at least one 1,2; 1,3; 1,4; and/or 1,6 glycosidic bond. Oligosaccharides may be linear or branched. Oligosaccharides may have one or more glycosidic bonds that are in alpha-configurations and/or one or more glycosidic bonds that are in beta-configurations.

Pharmaceutical Composition: As used herein, a “pharmaceutical composition” refers to a composition having pharmacological activity or other direct effect in the mitigation, treatment, or prevention of disease, and/or a finished dosage form or formulation thereof and is for human use. A pharmaceutical composition or pharmaceutical preparation is typically produced under good manufacturing practices (GMP) conditions. Pharmaceutical compositions or preparations may be sterile or non-sterile. If non-sterile, such pharmaceutical compositions or preparations typically meet the microbiological specifications and criteria for non-sterile pharmaceutical products as described in the U.S. Pharmacopeia (USP) or European Pharmacopoeia (EP). Any oligosaccharide composition described herein may be formulated as a pharmaceutical composition.

Subject: As used herein, the term “subject” refers to a human subject or patient. Subjects may include a newborn (a preterm newborn, a full-term newborn), an infant up to one year of age, young children (e.g., 1 yr to 12 yrs), teenagers, (e.g., 13-19 yrs), adults (e.g., 20-64 yrs), and elderly adults (65 yrs and older). In some embodiments, a subject is of a pediatric population, or a subpopulation thereof, including neonates (birth to 1 month), infants (1 month to 2 years), developing children (2-12 years), and adolescents (12-16 years). In some embodiments, a subject is a healthy subject. In some embodiments, a subject is a patient having decreased levels of SCFAs, e.g., butyrate, propionate, and/or acetate, relative to a healthy subject. In some embodiments, the subject has an autoimmune and/or allergic disorder (e.g., chronic inflammatory disorder). In some embodiments, the subject has an inflammatory bowel disease. In some embodiments, the subject has ulcerative colitis (UC), Crohn's disease (CD), granulomatous colitis, indeterminate colitis, diversion colitis, pouchitis, Behcet's disease, microscopic colitis, diverticulosis-associated colitis, collagenous colitis, lymphocytic colitis, or pediatric-onset inflammatory bowel disease. In some embodiments, the subject has decreased levels of commensal bacteria (e.g., Parabacteroides and Bacteroides), relative to a healthy subject. In some embodiments, the subject has increased levels of pathogenic bacteria (e.g., Enterobacteriaceae), relative to a healthy subject. In some embodiments, the subject has an elevated ratio of pathogenic bacteria relative to commensal bacteria, compared to a healthy subject. In some embodiments, the subject is between 20 and 70 years of age, between 20 and 60 years of age, between 25 and 60 years of age, or between 25 and 55 years of age. In some embodiments, the subject has at least one comorbidity, e.g., in addition to inflammatory bowel disease (e.g., UC, CD), such as, e.g., an autoimmune condition, e.g., rheumatoid arthritis. In one embodiment, the subject presents with mild disease (e.g., mild UC). For example, the subject presents with a Mayo score/disease activity index (DAI) for ulcerative colitis of 2-4, e.g., with less than 4 stools per day, with or without blood and limited erythema in superficial mucosa. In one embodiment, the subject presents with moderate disease (e.g., moderate UC). For example, the subject presents with a Mayo score/disease activity index (DAI) for ulcerative colitis of 5-7, e.g., with 5 or more stools per day and mild, but increasing anemia due to diarrhea and moderate ulceration of colon in endoscopy. In one embodiment, the subject presents with severe disease (e.g., severe UC). For example, the subject presents with a Mayo score/disease activity index (DAI) for ulcerative colitis of 8-9, e.g., with 8 or more stools per day of profuse bloody diarrhea, with endoscopy revealing loss of mucosal vascular markings. In one embodiment, the subject presents with mild to moderate disease (e.g., mild to moderate UC). In one embodiment, the subject presents with moderate to severe disease (e.g., moderate to severe UC).

Treatment and Treating: As used herein, the terms “treating” and “treatment” refer to the administration of a composition to a subject (e.g., a symptomatic subject afflicted with an adverse condition, disorder, or disease) so as to affect a reduction in severity and/or frequency of a symptom, eliminate a symptom and/or its underlying cause, and/or facilitate improvement or remediation of damage, and/or preventing an adverse condition, disorder, or disease in an asymptomatic subject who is susceptible to a particular adverse condition, disorder, or disease, or who is suspected of developing or at risk of developing the condition, disorder, or disease. In some embodiments, treating a subject with an oligosaccharide composition modulates, e.g., increase the relative or absolute levels of short chain fatty acids (SCFAs), e.g., butyrate, propionate, and/or acetate, in the subject. In some embodiments, treating a subject with an oligosaccharide composition reduces the severity of an autoimmune and/or allergic disorder (e.g., chronic inflammatory disorder). In some embodiments, treating a subject with an oligosaccharide composition reduces the severity of an inflammatory bowel diseases (e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease). In some embodiments, treating a subject with an oligosaccharide composition increases the quality of life of a person having or suspected of having an autoimmune and/or allergic disorder (e.g., chronic inflammatory disorder, e.g., an inflammatory bowel diseases, e.g., ulcerative colitis or Crohn's disease). In some embodiments, treating a subject with an oligosaccharide composition decreases the number and/or severity of symptoms (e.g., diarrhea, fever, fatigue, abdominal pain, bloody stool, inflammation, weight loss) of a person having or suspected of having an autoimmune and/or allergic disorder (e.g., chronic inflammatory disorder, e.g., an inflammatory bowel diseases, e.g., ulcerative colitis or Crohn's disease). In some embodiments, treating a subject with an oligosaccharide composition prevents the worsening, progression or onset of an autoimmune and/or allergic disorder (e.g., chronic inflammatory disorder, e.g., an inflammatory bowel diseases, e.g., ulcerative colitis or Crohn's disease). In some embodiments, treating a subject with an oligosaccharide composition reduces the number and/or rate of relapses of symptoms of a chronic inflammatory disorder (e.g., an inflammatory bowel diseases, e.g., ulcerative colitis or Crohn's disease). In some embodiments, treating a population of subjects with an oligosaccharide composition increases the average quality of life of treated persons having or suspected of having an autoimmune and/or allergic disorder (e.g., chronic inflammatory disorder, e.g., an inflammatory bowel diseases, e.g., ulcerative colitis or Crohn's disease). In some embodiments, treating a population of subjects with an oligosaccharide composition decreases the average number and/or severity of symptoms (e.g., diarrhea, fever, fatigue, abdominal pain, bloody stool, inflammation, weight loss) of treated persons having or suspected of having an autoimmune and/or allergic disorder (e.g., chronic inflammatory disorder, e.g., an inflammatory bowel diseases, e.g., ulcerative colitis or Crohn's disease). In some embodiments, treating a subject with an oligosaccharide composition results in at least a 5% improvement (e.g., at least a 10%, 15%, 20%, 25%, 30%, 35%, 40%, 50% improvement) in one or more of: number of symptoms, severity of symptoms, severity of the disease, and progression of the disease being treated, relative to a control or a standard of care treatment. For example, clinical remission of symptoms, e.g., after 10 weeks, 26 weeks, or 52 weeks (e.g., after commencement of treatment). Treatment may be assessed by measuring one or more disease associated biomarkers, including biomarkers associated with inflammation such as fecal calprotectin, fecal lactoferrin, and fecal lipocalin. Alternatively or in addition, treatment can be assessed by assessing mucosal healing (e.g., gut lining). Alternatively or in addition, treatment can be assessed by assessing quality of life (QoL), e.g., using the 32-item Inflammatory Bowel Disease Questionnaire (IBDQ-32), which assesses, e.g., bowel symptoms, emotional health, systemic systems and social function of the subject. In one embodiment, the QoL score can be self-reported. Treatment may also be assessed using the Simple Clinical Colitis Activity Index (SCCAI) composite score (e.g., as reported in Walmsley, R S; Ayres, R C S; Pounder, R E; Allan, R N (1998). “A simple clinical colitis activity index”. Gut. 43 (1): 29-32.)

### II. Oligosaccharide Compositions

Provided herein are oligosaccharide compositions, and their methods of use for lowering inflammation in a human subject.

In one aspect, oligosaccharide compositions are provided herein that comprise a plurality of oligosaccharides selected from Formula (I)

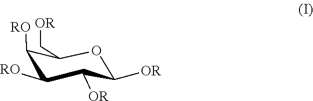

wherein R in Formula (I) is independently selected from hydrogen, and Formulae (Ia), (Ib), (Ic), (Id):

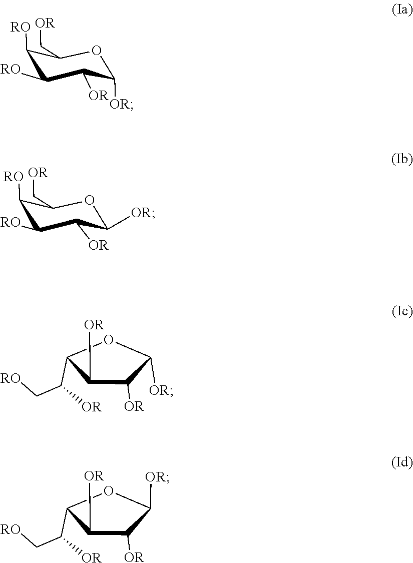

wherein R in Formulae (la), (Ib), (Ic), and (Id) is independently defined as above in Formula (I).

In some embodiments, oligosaccharide compositions are produced by a process that initially involves heating a preparation comprising galactose monomers to a temperature in a range of 100° C. to 160° C., 100° C. to 120° C., 110° C. to 130° C., 120° C. to 140° C., 130° C. to 150° C., or about 135° C. Heating may be performed under agitation conditions. Heating may comprise gradually increasing the temperature (e.g., from room temperature) to about 130° C., about 135° C. about 140° C. about 145° C., or about 150° C. under suitable conditions to achieve homogeneity and uniform heat transfer.

An acid catalyst comprising positively charged hydrogen ions is added to the preparation (e.g., before heating). In some embodiments, the acid catalyst is a solid catalyst. In some embodiments, the catalyst is a strong acid cation exchange resin having one or more physical and chemical properties according to Table 1. In some embodiments, the catalyst comprises >3.0 mmol/g sulfonic acid moieties and <1.0 mmol/gram cationic moieties. In certain embodiments, the catalyst has a nominal moisture content of 45-50 weight percent. In some embodiments, the catalyst is a soluble catalyst, e.g., an organic acid catalyst. In some embodiments, the catalyst is citric acid, acetic acid, butyric acid or propionic acid. In certain embodiments, the catalyst is added at the same time as the galactose monomers.

TABLE 1
Non-Limiting Example of Strong Acid
Cation Exchange Resin Properties

| Physical Form | | Amber translucent |
|---|---|---|
| | | spherical beads |
| Matrix | | Styrene-DVB, gel |
| Function group | | Sulfonic acid |
| Ionic form as shipped | | H+ form |
| Total volume capacity, min. | eq/L | 1.8 |
| | kgr/ft³ as | 39.3 |
| | CaCO₃ | |
| Moisture retention capacity | % | 50-56 |
| Particle size | | |
| Uniformity coefficient, max. | | 1.1 |
| Harmonic mean diameter | μm | 600 ± 50 |
| Whole uncracked beads | % | 95-100 |
| Total swelling (Na⁺ → H⁺) | | 8 |
| Particle density | g/mL | 1.2 |
| Shipping density | g/L | 800 |
| | lbs/ft³ | 50 |

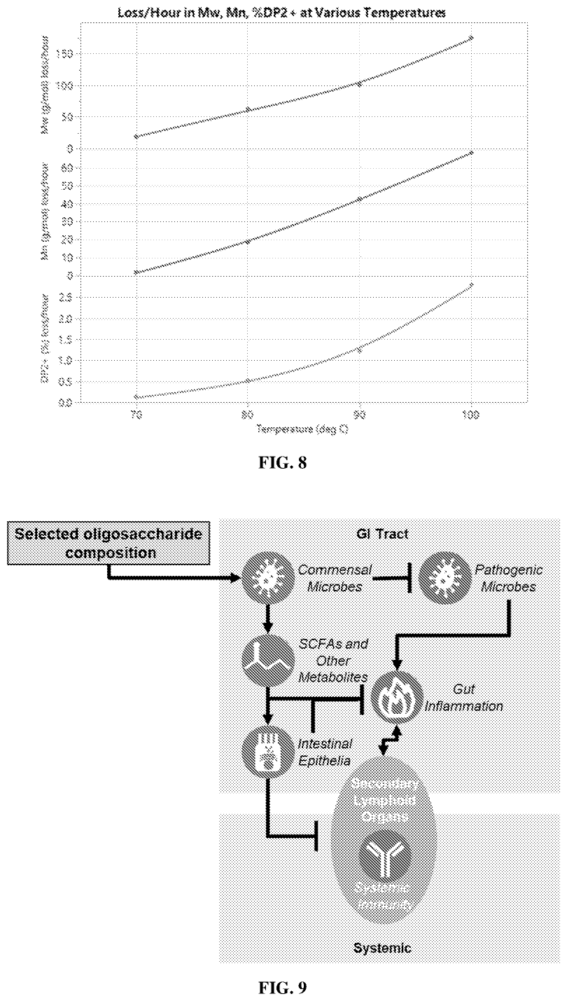

In some embodiments, after loading of the catalyst with the preparation, the resultant reaction mixture is held at atmospheric pressure and at a temperature in a range of 100° C. to 160° C., 100° C. to 120° C., 110° C. to 130° C., 120° C. to 140° C., 130° C. to 150° C., or about 135° C. under conditions that promote acid catalyzed oligosaccharide formation. In some embodiments, once the weight percent of total galactose monomer content in the oligosaccharide composition is in a range of 2-14% (optionally 2-5%, 4-8%, 5-13%, 7-10%, 7-11%, 9-14%, or 8-12%), the reaction mixture is quenched. Quenching typically involves using water (e.g., deionized water) to dilute the reaction mixture, and gradually decrease the temperature of the reaction mixture to 55° C. to 95° C. In some embodiments, the water used for quenching is about 95° C. The water may be added to the reaction mixture under conditions sufficient to avoid solidifying the mixture. In certain embodiments, water may be removed from the reaction mixture by evaporation. In some embodiments, the reaction mixture may contain 50-55 weight percent dissolved solids. In some embodiments, quenching should be performed in a timely manner in accordance with the disclosure of FIG. 8 and Example 19, which demonstrates the stability of the selected oligosaccharide composition at various elevated temperatures.

Finally, to obtain a purified oligosaccharide composition, the composition is typically by diluting the quenched reaction mixture with water to a concentration of about 35-60 weight percent (optionally 35-50 weight percent) and a temperature of below about 85° C. (e.g., room temperature) and then passing the mixture through a filter or a series of chromatographic resins. In some embodiments, the final, purified oligosaccharide composition is obtained by by diluting the quenched reaction mixture with water to a concentration of about 35-60 weight percent (optionally 35-50 weight percent) and a temperature of below about 85° C. (e.g., room temperature) without the use of any chromatographic resins. In some embodiments, the composition is separated from the acid catalyst. In certain embodiments, the filter used is a 0.45 μm filter. Alternatively, a series of chromatographic resins may be used and generally involves a cationic exchange resin, an anionic exchange resin, and/or a decolorizing polymer resin. In some embodiments, any or all of the types of resins may be used one or more times in any order. In some embodiments, the oligosaccharide composition comprises water at a level below that which is necessary for microbial growth upon storage at room temperature.

In some embodiments, the oligosaccharide composition is produced by a large-scale process (e.g., greater than 50 L scale, 500-5000 L, 1000-5000 L, 1000-4000 L, 1000-3000 L, 1500-3000 L, e.g., the size of the reaction reactor). In some embodiments, a large-scale process for producing the oligosaccharide composition is a 50 L process (e.g., a 50 L reactor), e.g., as described by Example 12. In some embodiments, a large-scale process for producing the oligosaccharide composition is a 2000 L process (e.g., a 2000 L reactor), e.g., as described by Example 13. A skilled person in the art would understand how to modify the large-scale processes described herein (e.g., as described by Examples 12 and 13) based on available reactors and instruments available to them (e.g., choice of size, shape, material, etc. for reactors, agitators, impellers, motors, etc.). For example, modifications to the large-scale process described by Example 13 (2000 L scale) could involve altering the temperature increase (e.g., quickening or slowing the temperature increase) from the boiling point (e.g., about 112° C.) to the reaction maintenance temperature (e.g., about 130° C.); and/or altering the agitation rate in a step-wise fashion (e.g., to reduce power usage by the reactor and its motor), e.g., to allow for increased viscosity of the reaction material.

In certain embodiments, the mean degree of polymerization of all oligosaccharides is in a range of 11-19, optionally 13-17. In some embodiments, the oligosaccharide composition comprises water in a range of 45-55 weight percent. In some embodiments, the oligosaccharide composition comprises oligosaccharides that have a MWw (weight-average molecular weight) (g/mol) in a range of 1900-2800, optionally 2214-2715. In some embodiments, the oligosaccharide composition comprises oligosaccharides that have a MWw (weight-average molecular weight) (g/mol) in a range of 2070-3090. In some embodiments, the oligosaccharide composition comprises oligosaccharides that have a MWn (number-average molecular weight) (g/mol) in a range of 1050-1250, optionally 1095-1201. In some embodiments, the oligosaccharide composition comprises oligosaccharides that have a MWn (number-average molecular weight) (g/mol) in a range of 1110-1350. In some embodiments, a solution comprising the oligosaccharide composition has a pH in a range of 2.50-3.50. In some embodiments, a solution comprising the oligosaccharide composition has a pH in a range of 2.00-5.00, optionally 2.00-4.80. In some embodiments, the oligosaccharide composition comprises oligomers having a degree of polymerization of at least two (DP2+) in a range of 89-94 weight percent. In some embodiments, the oligosaccharide composition comprises oligomers having a degree of polymerization of at least two (DP2+) in a range of 85-95 weight percent, optionally 88-90 weight percent. In some embodiments, the oligosaccharide composition comprises oligomers having a degree of polymerization of at least two (DP2+) in a range of 80-98 weight percent.

Further, in some embodiments, oligosaccharide compositions may be de-monomerized. In some embodiments, de-monomerization involves the removal of residual saccharide monomers. In some embodiments, de-monomerization is performed using chromatographic resin. Accordingly, in some embodiments, different compositions can be prepared depending upon the percent of monomer present. In some embodiments, oligosaccharide compositions are de-monomerized to a monomer content of about 1%, about 3%, about 5%, about 10%, or about 15%. In some embodiments, oligosaccharide composition are de-monomerized to a monomer content of about 1-3%, about 3-6%, about 5-8%, about 7-10%, or about 10-15%. In one embodiment, the oligosaccharide composition is de-monomerized to a monomer content of less than 1%. In one embodiment, the oligosaccharide composition is de-monomerized to a monomer content between about 7% and 10%. In one embodiment, the oligosaccharide composition is de-monomerized to a monomer content between about 0.1% and less than 2%. In one embodiment, the oligosaccharide compositions is de-monomerized to a monomer content between about 1% and 3%. In one embodiment, de-monomerization is achieved by osmotic separation. In a second embodiment de-monomerization is achieved by tangential flow filtration (TFF). In a third embodiment de-monomerization is achieved by ethanol precipitation.

In some embodiments, oligosaccharide compositions with different monomer contents may also have different measurements for total dietary fiber, moisture, total dietary fiber (dry basis), or percent Dextrose Equivalent (DE). In some embodiments, total dietary fiber is measured according to the methods of AOAC 2011.25. In some embodiments, moisture is measured by using a vacuum oven at 60° C. In some embodiments, total dietary fiber is (dry basis) is calculated. In some embodiments, the percent DE is measured according to the Food Chemicals Codex (FCC).

In some embodiments, the oligosaccharide compositions have a total dietary fiber content of 58-94 percent (on dry basis). In some embodiments, the oligosaccharide compositions have a total dietary fiber content of 65-87 percent (on dry basis). In some embodiments, the oligosaccharide compositions have a total dietary fiber content of 73-81 percent (on dry basis). In some embodiments, the oligosaccharide compositions have a total dietary fiber content of 50-80, 55-80, 60-80, 50-70, 55-70, 60-70, 50-65, 55-65, or 60-65 percent (on dry basis). In some embodiments, the oligosaccharide compositions have a total dietary fiber content of about 50, about 55, about 58, about 60, about 62, or about 65 percent (on dry basis).

In some embodiments, the oligosaccharide compositions have a total soluble dietary fiber content of 58-94 percent (on dry basis). In some embodiments, the oligosaccharide compositions have a total soluble dietary fiber content of 65-87 percent (on dry basis). In some embodiments, the oligosaccharide compositions have a total soluble dietary fiber content of 73-81 percent (on dry basis). In some embodiments, the oligosaccharide compositions have a total soluble dietary fiber content of 50-80, 55-80, 60-80, 50-70, 55-70, 60-70, 50-65, 55-65, or 60-65 percent (on dry basis). In some embodiments, the oligosaccharide compositions have a total soluble dietary fiber content of about 50, about 55, about 58, about 60, about 62, or about 65 percent (on dry basis).

In some embodiments, the oligosaccharide compositions have a total reducing sugar content (Dextrose Equivalence (DE) (dry solids)) of 5-50 percent.

In some embodiments, production of oligosaccharide compositions according to methods provided herein can be performed in a batch process or a continuous process. For example, in one embodiment, oligosaccharide compositions are produced in a batch process, where the contents of the reactor are subjected to agitation conditions (e.g., continuously mixed or blended), and all or a substantial amount of the products of the reaction are removed (e.g., isolated and/or recovered).

In certain embodiments, the methods of using the catalyst are carried out in an aqueous environment. One suitable aqueous solvent is water, which may be obtained from various sources. Generally, water sources with lower concentrations of ionic species (e.g., salts of sodium, phosphorous, ammonium, or magnesium) may be used, in some embodiments, as such ionic species may reduce effectiveness of the catalyst. In some embodiments where the aqueous solvent is water, the water has less than 10% of ionic species (e.g., salts of sodium, phosphorous, ammonium, magnesium). In some embodiments where the aqueous solvent is water, the water has a resistivity of at least 0.1 megaohm-centimeters, of at least 1 megaohm-centimeters, of at least 2 megaohm-centimeters, of at least 5 megaohm-centimeters, or of at least 10 megaohm-centimeters.

In some embodiments, as reactions of methods provided herein progress, water (such as evolved water) is produced with each glycosidic coupling of the one or more saccharide monomer. In certain embodiments, the methods described herein may further include monitoring the amount of water present in the reaction mixture and/or the ratio of water to monomer or catalyst over a period of time. Thus, in some embodiments, the water content of the reaction mixture may be altered over the course of the reaction, for example, removing evolved water produced. Appropriate methods may be used to remove water (e.g., evolved water) in the reaction mixture, including, for example, by evaporation, such as via distillation. In some embodiments, the method comprises including water in the reaction mixture. In certain embodiments, the method comprises removing water from the reaction mixture through evaporation,

In certain embodiments, the preparation is loaded with an acid catalyst comprising positively charged hydrogen ions. In some embodiments, an acid catalyst is a solid catalyst (e.g., Dowex Marathon C). In some embodiments, an acid catalyst is a soluble catalyst (e.g., citric acid).

In some embodiments, the molar ratio of positively charged hydrogen ions to total galactose monomer content is in an appropriate range. In some embodiments, the molar ratio of positively charged hydrogen ions to total galactose monomer content is in a range of 0.01 to 0.1, 0.02 to 0.08, 0.03 to 0.06, or 0.05 to 0.06. In some embodiments, the molar ratio of positively charged hydrogen ions to total galactose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3.

In some embodiments, the molar ratio of soluble acid catalyst (e.g., citric acid catalyst) to total galactose monomer content is in an appropriate range. In some embodiments, the molar ratio of soluble acid catalyst (e.g., citric acid catalyst) to total galactose monomer content is in a range of 0.01 to 0.1, 0.02 to 0.08, 0.03 to 0.06, or 0.05 to 0.06. In some embodiments, the molar ratio of soluble acid catalyst (e.g., citric acid catalyst) to total galactose monomer content is in a range of 0.003 to 0.01, 0.005 to 0.02, 0.01 to 0.02, 0.01 to 0.03, 0.02 to 0.03, 0.02 to 0.04, 0.03 to 0.05, 0.03 to 0.08, 0.04 to 0.07, 0.05 to 0.1, 0.05 to 0.2, 0.1 to 0.2, 0.1 to 0.3, or 0.2 to 0.3.

In some embodiments, water is added to the reaction mixture to quench the reaction by bringing the temperature of the reaction mixture to 100° C. or below. In some embodiments, the water used for quenching is deionized water. In some embodiments, the water used for quenching is USP water. In some embodiments, the water has a temperature of about 60° C. to about 100° C. In certain embodiments, the water used for quenching is about 95° C. In some embodiments, the water is added to the reaction mixture under conditions sufficient to avoid solidifying the mixture.

The viscosity of the reaction mixture may be measured and/or altered over the course of the reaction. In general, viscosity refers to a measurement of a fluid's internal resistance to flow (e.g., “thickness”) and is expressed in centipoise (cP) or pascal-seconds. In some embodiments, the viscosity of the reaction mixture is between about 100 cP and about 95,000 cP, about 5,000 cP and about 75,000 cP, about 5,000 and about 50,000 cP, or about 10,000 and about 50,000 cP. In certain embodiments, the viscosity of the reaction mixture is between about 50 cP and about 200 cP,

In some embodiments, oligosaccharide compositions provided herein may be subjected to one or more additional processing steps. Additional processing steps may include, for example, purification steps. Purification steps may include, for example, separation, demonomerization, dilution, concentration, filtration, desalting or ion-exchange, chromatographic separation, or decolorization, or any combination thereof.

In certain embodiments, the methods described herein further include a dilution step. In some embodiments, deionized water is used for dilution. In certain embodiments, USP water is used for dilution. In certain embodiments, after dilution, the oligosaccharide composition comprises water in a range of about 5-75, 25-65, 35-65, 45-55, or 47-53 weight percent. In certain embodiments, after dilution, the oligosaccharide composition comprises water in a range of about 35-65 weight percent. In certain embodiments, after dilution, the oligosaccharide composition comprises water in a range of about 40-50 weight percent.

In some embodiments, the methods described herein further include a decolorization step. The one or more oligosaccharide compositions produced may undergo a decolorization step using appropriate methods, including, for example, treatment with an absorbent, activated carbon, chromatography (e.g., using ion exchange resin), and/or filtration (e.g., microfiltration).

In some embodiments, the one or more oligosaccharide compositions produced are contacted with a material to remove salts, minerals, and/or other ionic species. For example, in certain embodiments, the one or more oligosaccharide compositions produced are flowed through an anionic exchange column. In other embodiments, oligosaccharide compositions produced are flowed through an anionic/cationic exchange column pair.

In some embodiments, the methods described herein may further include a concentration step. For example, in some embodiments, the oligosaccharide compositions may be subjected to evaporation (e.g., vacuum evaporation) to produce a concentrated oligosaccharide composition. In other embodiments, the oligosaccharide compositions may be subjected to a spray drying step to produce an oligosaccharide powder. In certain embodiments, the oligosaccharide compositions may be subjected to both an evaporation step and a spray drying step. In some embodiments, the oligosaccharide compositions be subjected to a lyophilization (e.g., freeze drying) step to remove water and produce powdered product.

In some embodiments, the methods described herein further include a fractionation step. Oligosaccharide compositions prepared and purified may be subsequently separated by molecular weight using any method known in the art, including, for example, high-performance liquid chromatography, adsorption/desorption (e.g., low-pressure activated carbon chromatography), or filtration (for example, ultrafiltration or diafiltration). In certain embodiments, oligosaccharide compositions are separated into pools representing 60%, 65%, 70%, 75%, 80%, 85%, 90%, 95%, 97%, 98%, or greater than 98% short (about DP1-2), medium (about DP3-10), long (about DP11-18), or very long (about DP>18) species.

In certain embodiments, prepared oligosaccharide compositions are fractionated by adsorption onto a carbonaceous material and subsequent desorption of fractions by washing the material with mixtures of an organic solvent in water at a concentration of 1%, 5%, 10%, 20%, 50%, or 100%. In one embodiment, the adsorption material is activated charcoal. In another embodiment, the adsorption material is a mixture of activated charcoal and a bulking agent such as diatomaceous earth or Celite 545 in 5%, 10%, 20%, 30%, 40%, or 50% portion by volume or weight.

In further embodiments, prepared oligosaccharide compositions are separated by passage through a high-performance liquid chromatography system. In certain variations, prepared oligosaccharide compositions are separated by ion-affinity chromatography, hydrophilic interaction chromatography, or size-exclusion chromatography including gel-permeation and gel-filtration.

In some embodiments, catalyst is removed by filtration. In certain embodiments, a 0.45 μm filter is used to remove catalyst during filtration. In other embodiments, low molecular weight materials are removed by filtration methods. In certain variations, low molecular weight materials may be removed by dialysis, ultrafiltration, diafiltration, or tangential flow filtration. In certain embodiments, the filtration is performed in static dialysis tube apparatus. In other embodiments, the filtration is performed in a dynamic flow filtration system. In other embodiments, the filtration is performed in centrifugal force-driven filtration cartridges. In certain embodiments, the reaction mixture is cooled to below about 85° C. before filtration.

In certain embodiments, the mean degree of polymerization of all oligosaccharides is in a range of 11-19. In certain embodiments, the mean degree of polymerization of all oligosaccharides is in a range of 10-16. In certain embodiments, the mean degree of polymerization of all oligosaccharides is in a range of 13-17. In certain embodiments, the mean degree of polymerization of all oligosaccharides is about 15. In some embodiments, the mean degree of polymerization of all oligosaccharides is in a range of 5-20, 6-19, 11-16, 12-18, 10-17, 7-15, 7-12, 7-10, 7-8, 9-10, 10-11, 11-11, 11-15, 12-13, 12-14 13-14, 14-15, 15-16, 17-18, 15-20, 3-8, 4-7, or 5-6. In some embodiments, the mean degree of polymerization (DP) of all oligosaccharides is about 6, about 7, about 8, about 9, about 10, about 11, about 12, about 13, about 14, about 15, about 16, about 17, about 18, about 19, about 20, or about 21.

In certain embodiments, the weight percent of galactose monomer in the oligosaccharide composition is in a range of 6-12. In certain embodiments, the weight percent of galactose monomer in the oligosaccharide composition is in a range of 8-10. In certain embodiments, the weight percent of galactose monomer in the oligosaccharide composition is in a range of 5-13. In certain embodiments, the weight percent of galactose monomer in the oligosaccharide composition is in a range of 6-7.

In some embodiments, the oligosaccharide composition is a mixture of polymers comprising a formula of H—[C₆H₉₋₁₁O₅]ₙ—OH, where the total number of monomer units in a single polymer of the mixture ranges from 2 to approximately 60 (n=2-60), with a mean value for the mixture of approximately 15.1 monomer units. Each monomer unit may be unsubstituted, singly, doubly, or triply substituted with another galactose unit by any glycosidic isomer.

In some embodiments, the oligosaccharide composition comprises water in a range of 5-75 weight percent. In some embodiments, the oligosaccharide composition comprises water in a range of 25-65 weight percent. In some embodiments, the oligosaccharide composition comprises water in a range of 35-65 weight percent. In some embodiments, the oligosaccharide composition comprises water in a range of 45-55 weight percent.

In some embodiments, the oligosaccharide composition comprises oligosaccharides that have a MWw (g/mol) in a range of 2214-2715. In some embodiments, the oligosaccharide composition comprises oligosaccharides that have a MWw (g/mol) in a range of 1816-3070. In some embodiments, the oligosaccharide composition comprises oligosaccharides that have a MWw (g/mol) in a range of 1400-3500, 1800-3100, 1500-2000, 1700-2200, 1900-2400, 2100-2600, 2300-2800, 2500-3000, or 2700-3200.

In some embodiments, the oligosaccharide composition comprises oligosaccharides that have a MWn (g/mol) in a range of 1095-1201. In some embodiments, the oligosaccharide composition comprises oligosaccharides that have a MWn (g/mol) in a range of 1011-1299. In some embodiments, the oligosaccharide composition comprises oligosaccharides that have a MWn (g/mol) in a range of 900-1100, 1000-1200, 1100-1400, 1200-1500, 1300-1600, or 1400-1700.

In some embodiments, a solution comprising the oligosaccharide composition has a pH in a range of 1.50-6.00. In some embodiments, a solution comprising the oligosaccharide composition has a pH in a range of 1.50-5.00. In some embodiments, a solution comprising the oligosaccharide composition has a pH in a range of 2.00-4.00. In some embodiments, a solution comprising the oligosaccharide composition has a pH in a range of 2.50-3.50.

In some embodiments, the oligosaccharide composition comprises oligosaccharides that have a degree of branching in a range of about 13% to about 30%. In some embodiments, the oligosaccharide composition comprises oligosaccharides that have a degree of branching in a range of about 15% to about 26%. In some embodiments, the oligosaccharide composition comprises oligosaccharides that have a degree of branching in a range of 20% to 21%. In some embodiments, the oligosaccharide composition comprises oligosaccharides that have a degree of branching in a range of 5-50%, 5-40%, 5-30%, 5-20%, 5-15%, 10-50%, 10-40%, 10-30%, 10-25%, 15-30%, or 15-20%.

In some embodiments, the oligosaccharide composition comprises oligomers having two or more repeat units (DP2+) in a range of 80-100 weight percent. In some embodiments, the oligosaccharide composition comprises oligomers having two or more repeat units (DP2+) in a range of 87-96 weight percent. In some embodiments, the oligosaccharide composition comprises oligomers having two or more repeat units (DP2+) in a range of 88-94 weight percent. In some embodiments, the oligosaccharide composition comprises oligomers having two or more repeat units (DP2+) in a range of 90-92 weight percent. In some embodiments, the oligosaccharide composition comprises oligomers having two or more repeat units (DP2+) in a range of 80-85, 85-87, 86-88, 87-90, 88-91, 89-92, 90-93, 91-94, 92-95, 93-96, or 95-98 weight percent.

In some embodiments, the oligosaccharide composition has a polydispersity index (PDI) of 1.8-2.4. In some embodiments, the oligosaccharide composition has a polydispersity index (PDI) of 2.0-2.3. In some embodiments, the oligosaccharide composition has a PDI of 1.0-1.2, 1.2-1.3, 1.3-1.4, 1.4-1.5, 1.5-1.6, 1.7-1.8, 1.8-2.0, 2.0-2.2, 2.2-2.4, or 2.4-2.6. In some embodiments, the oligosaccharide composition has a PDI of about 1.6, about 1.7, about 1.8, about 1.9, about 2.0, about 2.1, about 2.2, about 2.3, or about 2.4.

In some embodiments, the MWw, MWn, PDI, monomer content (DP1) and/or DP2+ values of oligosaccharides in an oligosaccharide composition are determined using the size exclusion chromatography method described in Example 6.

In some embodiments, the oligosaccharide composition comprises oligomers having at least three linked monomer units (DP3+) in a range of 80-85, 85-87, 86-88, 87-90, 88-91, 89-92, 90-93, 91-94, or 92-95 weight percent.

In some embodiments, the oligosaccharide composition comprises 6.4% to 11.4% monomer (DP1). In some embodiments, the oligosaccharide composition comprises 5% to 10%, 7% to 12%, 11% to 18%, 10% to 15%, or 12% to 17% monomer (DP1). In some embodiments, the oligosaccharide composition comprises 88.6% to 94.6% oligomers having at least two linked monomer units (DP2+). In some embodiments, the oligosaccharide composition comprises 80% to 81%, 81% to 82%, 82% to 83%, 84% to 85%, 85% to 86%, 86% to 87%, 87% to 88%, 88% to 90%, or 89% to 95% oligomers having at least two linked monomer units (DP2+). In some embodiments, the oligosaccharide composition comprises 84% to 85%, 85% to 86%, 86% to 87%, 87% to 88%, or 88% to 90% oligomers having at least three linked monomer units (DP3+).

In some embodiments, the oligosaccharide composition comprises less than 0.10% total impurities (excluding monomer). In some embodiments, the oligosaccharide composition comprises less than 0.05% total impurities (excluding monomer). In some embodiments, the oligosaccharide composition comprises less than 0.20%, 0.15%, 0.10%, or 0.05% total impurities (excluding monomer). In some embodiments, the oligosaccharide composition comprises less than 0.10% w/w glucuronic acid, less than 0.10% w/w lactic acid, less than 0.10% w/w formic acid, less than 0.10% w/w acetic acid, and less than 0.10% w/w hydroxymethylfurfural (HMF). In some embodiments, the oligosaccharide composition comprises undetectable levels of lactic acid, formic acid, levulinic acid and HMF. In some embodiments, the oligosaccharide composition comprises 0.19% w/w citric acid. In some embodiments, the oligosaccharide composition comprises 0.15-0.22% w/w, 0.10-1.00% w/w, 0.50-1.50% w/w, 1.00-2.00% w/w, 2.00-3.00% w/w, 2.00-2.50% w/w, or 2.50-3.00% w/w citric acid.

In some embodiments, the oligosaccharide composition comprises a MWw of 2214-2715, a MWn of 1095-1201, and/or a PDI of 2.0-2.3.

In certain embodiments, the oligosaccharide composition analyzed by NMR contains monosaccharide monomers (DP1), i.e., the DP1 component is not removed from the composition prior to NMR analysis. For example, in some embodiments, the oligosaccharide composition analyzed by NMR contains between 10%-25% DP1 monomers. In certain embodiments, the composition analyzed by NMR is de-monomerized, i.e., some or all of the DP1 component of the composition is removed prior to NMR analysis, e.g., by the method described in Example 8. For example, in some embodiments, the oligosaccharide composition analyzed by NMR contains between 0.05% to 10% DP1 monomers.

The oligosaccharide compositions described herein, and prepared according to the methods described herein, can be characterized and distinguished from prior art compositions using permethylation analysis. See, e.g., Zhao, Y., et al. ‘Rapid, sensitive structure analysis of oligosaccharides,’ PNAS Mar. 4, 1997 94 (5) 1629-1633; Kailemia, M. J., et al. ‘Oligosaccharide analysis by mass spectrometry: A review of recent developments,’ Anal Chem. 2014 Jan. 7; 86 (1): 196-212. Accordingly, in another aspect, oligosaccharide compositions are provided herein that comprise a plurality of oligosaccharides, e.g., that are minimally digestible in humans, the plurality of oligosaccharides comprising monomer radicals. The molar percentages of different types of monomer radicals in the plurality of oligosaccharides can be quantified using a permethylation assay as described in Example 9. The permethylation assay is performed on a de-monomerized sample of the composition.

In some embodiments, the plurality of oligosaccharides comprises two or more monomer radicals selected from radicals (1)-(20):
(1) t-galactofuranose monoradicals, representing 4.11-15.02 mol % (optionally 6.29-12.84 mol %) of monomer radicals in the plurality of oligosaccharides;
(2) t-galactopyranose monoradicals, representing 17.74-31.28 mol % (optionally 20.45-28.28 mol %) of monomer radicals in the plurality of oligosaccharides;
(3) 2-galactofuranose and/or 2-glucofuranose monoradicals, representing 2.48-3.70 mol % (optionally 2.73-3.46% mol %) of monomer radicals in the plurality of oligosaccharides;
(4) 3-galactopyranose monoradicals, representing 4.70-7.76 mol % (optionally 5.31-7.15 mol %) of monomer radicals in the plurality of oligosaccharides;
(5) 3-galactofuranose monoradicals, representing 3.05-4.58 mol % (optionally 3.36-4.28 mol %) of monomer radicals in the plurality of oligosaccharides;
(6) 2-galactopyranose monoradicals, representing 4.01-4.56 mol % (optionally 4.12-4.45 mol %) of monomer radicals in the plurality of oligosaccharides;
(7) 4-galactopyranose and/or 5-galactofuranose monoradicals, representing 4.24-6.28 mol % (optionally 4.65-5.87 mol %) of monomer radicals in the plurality of oligosaccharides;
(8) 2,3-galactofuranose diradicals, representing 0.30-0.95 mol % (optionally 0.43-0.82 mol %) of monomer radicals in the plurality of oligosaccharides;
(9) 6-galactofuranose monoradicals, representing 0.70-11.80 mol % (optionally 2.92-9.58 mol %) of monomer radicals in the plurality of oligosaccharides;
(10) 6-galactopyranose monoradicals, representing 12.07-20.76 mol % (optionally 13.81-19.02 mol %) of monomer radicals in the plurality of oligosaccharides;
(11) 3,4-galactopyranose and/or 3,5-galactofuranose and/or 2,3-galactopyranose diradicals, representing 1.22-2.19 mol % (optionally 1.41-1.99 mol %) of monomer radicals in the plurality of oligosaccharides;
(12) 2,4-glucopyranose and/or 2,5-glucofuranose and/or 2,4-galactopyranose and/or 2,5-galactofuranose diradicals, representing 0.77-1.32 mol % (optionally 0.88-1.21 mol %) of monomer radicals in the plurality of oligosaccharides;
(13) 2,3,4-galactopyranose and/or 2,3,5-galactofuranose triradicals, representing 0.09-0.32 mol % (optionally 0.14-0.28 mol %) of monomer radicals in the plurality of oligosaccharides;
(14) 3,6-galactofuranose diradicals, representing 1.49-2.46 mol % (optionally 1.69-2.27 mol %) of monomer radicals in the plurality of oligosaccharides;
(15) 4,6-galactopyranose and/or 5,6-galactofuranose diradicals, representing 3.49-5.70 mol % (optionally 3.93-5.26 mol %) of monomer radicals in the plurality of oligosaccharides;
(16) 3,6-galactopyranose and/or 2,6-galactofuranose diradicals, representing 3.54-6.52 mol % (optionally 4.14-5.93 mol %) of monomer radicals in the plurality of oligosaccharides;
(17) 2,6-galactopyranose diradicals, representing 1.65-3.32 mol % (optionally 1.98-2.99 mol %) of monomer radicals in the plurality of oligosaccharides.
(18) 3,4,6-galactopyranose and/or 3,5,6-galactofuranose and/or 2,3,6-galactofuranose triradicals, representing 0.65-1.94 mol % (optionally 0.91-1.68 mol %) of monomer radicals in the plurality of oligosaccharides;
(19) 2,3,6-galactopyranose and/or 2,4,6-galactopyranose and/or 2,5,6-galactofuranose triradicals, representing 0.01-4.14 mol % (optionally 0.01-3.10 mol %) of monomer radicals in the plurality of oligosaccharides; and/or
(20) 2,3,4,6-galactopyranose and/or 2,3,5,6-galactofuranose quadradicals, representing 0.01-0.44 mol % (optionally 0.01-0.28 mol %) of monomer radicals in the plurality of oligosaccharides.

In some embodiments, 14-30% of the total glycosidic bonds in an oligosaccharide composition are 1,2 glycosidic bonds. In some embodiments, 16.7-26.2% of the total glycosidic bonds in an oligosaccharide composition are 1,2 glycosidic bonds. In some embodiments, about 20% or about 21% of the total glycosidic bonds in an oligosaccharide composition are 1,2 glycosidic bonds. In some embodiments, about 8.7-33.6% (optionally about 12.4-28.0%) of the total glycosidic bonds in an oligosaccharide composition are 1,2 glycosidic bonds. In some embodiments, 15-50%, 15-40%, 15-30%, 15-20%, 20-40%, 20-30%, 25-50%, 25-30%, or 30-45% of the total glycosidic bonds in an oligosaccharide composition are 1,2 glycosidic bonds.

In some embodiments, about 15-32% of the total glycosidic bonds in an oligosaccharide composition are 1,3 glycosidic bonds. In some embodiments, about 21% or about 22% of the total glycosidic bonds in an oligosaccharide composition are 1,3 glycosidic bonds. In some embodiments, 17.4-27.8% of the total glycosidic bonds in an oligosaccharide composition are 1,3 glycosidic bonds. In some embodiments, about 11.0-34.4% (optionally 14.4-29.2%) of the total glycosidic bonds in an oligosaccharide composition are 1,3 glycosidic bonds. In some embodiments, 10-50%, 15-40%, 15-30%, 15-25%, 10-40%, 10-30%, 10-25%, 15-30%, or 15-20% of the total glycosidic bonds in an oligosaccharide composition are 1,3 glycosidic bonds.

In some embodiments, about 10-22% of the total glycosidic bonds in an oligosaccharide composition are 1,4 glycosidic bonds. In some embodiments, about 16% of the total glycosidic bonds in an oligosaccharide composition are 1,4 glycosidic bonds. In some embodiments. 11.9-19.7% of the total glycosidic bonds in an oligosaccharide composition are 1,4 glycosidic bonds. In some embodiments, 9.5-23.5% (optionally 11.4-20.4%) of the total glycosidic bonds in an oligosaccharide composition are 1,4 glycosidic bonds. In some embodiments, 5-35%, 10-30%, 10-25%, 10-20%, 5-20%, 5-15%, or 20-30% of the total glycosidic bonds in an oligosaccharide composition are 1,4 glycosidic bonds.

In some embodiments, about 24-57% of the total glycosidic bonds in an oligosaccharide composition are 1,6 glycosidic bonds. In some embodiments, about 39% or about 40% of the total glycosidic bonds in an oligosaccharide composition are 1,6 glycosidic bonds. In some embodiments, 29.4-50.1% of the total glycosidic bonds in an oligosaccharide composition are 1,6 glycosidic bonds. In some embodiments, 20.9-57.8% (optionally 27.6-50.2%) of the total glycosidic bonds in an oligosaccharide composition are 1,6 glycosidic bonds. In some embodiments, 25-60%, 25-50%, 25-40%, 30-60%, 30-50%, 30-40%, or 35-45% of the total glycosidic bonds in an oligosaccharide composition are 1,6 glycosidic bonds.

In some embodiments, an oligosaccharide composition comprises 26-49% total furanose. In some embodiments, an oligosaccharide composition comprises about 38% or about 44% total furanose. In some embodiments, an oligosaccharide composition comprises 19-71 (optionally 28-61%) total furanose. In some embodiments, an oligosaccharide composition comprises 15-75%, 15-65%, 15-50%, 15-40%, 20-40%, 25-35%, or 25-40% total furanose.

In some embodiments, the oligosaccharide composition comprises at least one galactofuranose or galactopyranose radical.

In some embodiments, an oligosaccharide composition is provided, comprising a plurality of oligosaccharides comprising monomer radicals (1)-(20) in the molar percentages shown in Table 2.

TABLE 2
Permethylation Data

| | Mean | | Mean |
|---|---|---|---|
| | mol | | mol |
| | % −3 | Mean | % +3 |
| | standard | mol | standard |
| Radicals | dev. | % | dev. |
| t-galactofuranose monoradicals | 6.29% | 9.56% | 12.84% |
| t-galactopyranose monoradicals | 20.45% | 24.51% | 28.58% |
| 2-galactofuranose and/or 2- | 2.73% | 3.09% | 3.46% |
| glucofuranose monoradicals | | | |
| 3-galactopyranose monoradicals | 5.31% | 6.23% | 7.15% |
| 3-galactofuranose monoradicals | 3.36% | 3.82% | 4.28% |
| 2-galactopyranose monoradicals | 4.12% | 4.29% | 4.45% |
| 4-galactopyranose and/or 5- | 4.65% | 5.26% | 5.87% |
| galactofuranose monoradicals | | | |
| 2,3-galactofuranose diradicals | 0.43% | 0.62% | 0.82% |
| 6-galactofuranose monoradicals | 2.92% | 6.25% | 9.58% |
| 6-galactopyranose monoradicals | 13.81% | 16.42% | 19.02% |
| 3,4-galactopyranose and/or 3,5- | 1.41% | 1.70% | 1.99% |
| galactofuranose and/or 2,3- | | | |
| galactopyranose diradicals | | | |
| 2,4-glucopyranose and/or 2,5- | 0.88% | 1.05% | 1.21% |
| glucofuranose and/or 2,4-galactopyranose | | | |
| and/or 2,5-galactofuranose diradicals | | | |
| 2,3,4-galactopyranose and/or 2,3,5- | 0.14% | 0.21% | 0.28% |
| galactofuranose triradicals | | | |
| 3,6-galactofuranose diradicals | 1.69% | 1.98% | 2.27% |
| 4,6-galactopyranose and/or 5,6- | 3.93% | 4.60% | 5.26% |
| galactofuranose diradicals | | | |
| 3,6-galactopyranose and/or 2,6- | 4.14% | 5.03% | 5.93% |
| galactofuranose diradicals | | | |
| 2,6-galactopyranose diradicals | 1.98% | 2.49% | 2.99% |
| 3,4,6-galactopyranose and/or 3,5,6- | 0.91% | 1.30% | 1.68% |
| galactofuranose and/or 2,3,6- | | | |
| galactofuranose triradicals | | | |
| 2,3,6-galactopyranose and/or 2,4,6- | 0.01% | 1.54% | 3.10% |
| galactopyranose and/or 2,5,6- | | | |
| galactofuranose triradicals | | | |
| 2,3,4,6-galactopyranose and/or 2,3,5,6- | 0.01% | 0.04% | 0.28% |
| galactofuranose quadradicals | | | |

Permethylation data that characterize individual batches of the selected oligosaccharide composition are described in Examples 9 and 16. A composite of these data described in Examples 9 and 16 are provided below to show averaged data with standard deviations (std. dev.) across all tested batches and replicates describing monomer radicals present in the selected oligosaccharide composition, as shown in Table 3.

TABLE 3
Permethylation Data (composite data)

| | Mean | Mean | | Mean | Mean |
|---|---|---|---|---|---|
| | mol % −5 | mol % −3 | | mol % +3 | mol % +5 |
| | standard | standard | Mean | standard | standard |
| Radicals | dev. | dev. | mol % | dev. | dev. |
| t-galactofuranose monoradicals | 2.52% | 5.06% | 8.87% | 12.67% | 15.21% |
| t-galactopyranose | 13.49% | 18.79% | 26.75% | 34.71% | 40.02% |
| monoradicals | | | | | |
| 2-galactofuranose and/or 2- | 0.64% | 1.48% | 2.73% | 3.99% | 4.82% |
| glucofuranose monoradicals | | | | | |
| 3-galactopyranose | 4.79% | 5.38% | 6.27% | 7.16% | 7.75% |
| monoradicals | | | | | |
| 3-galactofuranose | 2.22% | 2.78% | 3.62% | 4.47% | 5.03% |
| monoradicals | | | | | |
| 2-galactopyranose | 3.10% | 3.51% | 4.12% | 4.73% | 5.13% |
| monoradicals | | | | | |
| 4-galactopyranose and/or 5- | 3.99% | 4.57% | 5.43% | 6.30% | 6.87% |
| galactofuranose monoradicals | | | | | |
| 2,3-galactofuranose diradicals | 0.00% | 0.00% | 0.32% | 1.28% | 1.93% |
| 6-galactofuranose | 1.52% | 3.30% | 5.96% | 8.62% | 10.39% |
| monoradicals | | | | | |
| 6-galactopyranose | 11.64% | 13.76% | 16.94% | 20.12% | 22.24% |
| monoradicals | | | | | |
| 3,4-galactopyranose and/or | 0.68% | 1.18% | 1.92% | 2.66% | 3.15% |
| 3,5-galactofuranose and/or 2,3- | | | | | |
| galactopyranose diradicals | | | | | |
| 2,4-glucopyranose and/or 2,5- | 0.49% | 0.69% | 0.97% | 1.26% | 1.45% |
| glucofuranose and/or 2,4- | | | | | |
| galactopyranose and/or 2,5- | | | | | |
| galactofuranose diradicals | | | | | |
| 2,3,4-galactopyranose and/or | 0.00% | 0.00% | 0.18% | 0.48% | 0.67% |
| 2,3,5-galactofuranose | | | | | |
| triradicals | | | | | |
| 3,6-galactofuranose diradicals | 0.41% | 0.95% | 1.76% | 2.56% | 3.10% |
| 4,6-galactopyranose and/or | 3.60% | 4.01% | 4.62% | 5.24% | 5.65% |
| 5,6-galactofuranose diradicals | | | | | |
| 3,6-galactopyranose and/or | 2.20% | 3.17% | 4.63% | 6.09% | 7.06% |
| 2,6-galactofuranose diradicals | | | | | |
| 2,6-galactopyranose diradicals | 0.89% | 1.44% | 2.26% | 3.08% | 3.63% |
| 3,4,6-galactopyranose and/or | 0.68% | 0.91% | 1.26% | 1.61% | 1.85% |
| 3,5,6-galactofuranose and/or | | | | | |
| 2,3,6-galactofuranose | | | | | |
| triradicals | | | | | |
| 2,3,6-galactopyranose and/or | 0.00% | 0.04% | 1.34% | 2.64% | 3.51% |
| 2,4,6-galactopyranose and/or | | | | | |
| 2,5,6-galactofuranose | | | | | |
| triradicals | | | | | |
| 2,3,4,6-galactopyranose and/or | 0.00% | 0.00% | 0.03% | 0.22% | 0.35% |
| 2,3,5,6-galactofuranose | | | | | |
| quadradicals | | | | | |

In certain embodiments, the oligosaccharide compositions are free from monomer (e.g., de-monomerized). In other embodiments, the oligosaccharide compositions comprise monomer.

In some embodiments, an oligosaccharide composition is provided, comprising a plurality of oligosaccharides comprising or consisting essentially of monomer radicals (1)-(20), as described herein. In some embodiments, an oligosaccharide composition is provided, comprising a plurality of oligosaccharides comprising at least one (e.g., at least two, at least three, at least four, at least five, at least six, at least seven, at least eight, at least nine, or at least ten) monomer radical selected from radicals (1)-(20) with at least one (e.g., at least two, at least three, at least four, at least five, at least six, at least seven, at least eight, at least nine, or at least ten) corresponding molar percentage shown in Table 2. In some embodiments, an oligosaccharide composition is provided, comprising a plurality of oligosaccharides comprising or consisting of monomer radicals (1)-(20) with the molar percentages shown in Table 2.

The oligosaccharide compositions described herein, and prepared according to the methods described herein, can be characterized and distinguished from prior art compositions using two-dimensional heteronuclear NMR. Accordingly, in another aspect, oligosaccharide compositions are provided that comprise a plurality of oligosaccharides, e.g., that are minimally digestible in humans, the plurality of oligosaccharides being characterized by a multiplicity-edited gradient-enhanced 1H-¹³C heteronuclear single quantum correlation (HSQC) NMR spectrum comprising one or more of signals 1-11 of Table 4, wherein the spectrum is generated using a sample of the oligosaccharide composition having less than 2% monomer.

TABLE 4
HSQC NMR data

| | Center Position (ppm) | | Area under the curve (AUC) |
|---|---|---|---|
| Signal | ¹H | ¹³C | (% of total areas of signals 1-11) |
| 1 | 5.04 | 108.38 | 4.12-6.38 |
| 2 | 5.02 | 101.68 | 0.56-1.84 |
| 3 | 4.98 | 98.99 | 8.06-23,52 |
| 4 | 4.81 | 101.2 | 0.77-2.04 |
| 5 | 4.6 | 104.96 | 0.01-5.31 |
| 6 | 4.45 | 103.84 | 5.80-16.52 |
| 7 | 4.19 | 82.1 | 3.22-6.15 |
| 8 | 4.14 | 76.91 | 6.85-14.13 |
| 9 | 4.11 | 81.72 | 10.59-20.38 |
| 10 | 4.06 | 77.25 | 14.72-28.56 |
| 11 | 3.98 | 83.52 | 8.75-11.89 |

Accordingly, in another aspect, oligosaccharide compositions are provided that comprise a plurality of oligosaccharides, e.g., that are minimally digestible in humans, the plurality of oligosaccharides being characterized by a multiplicity-edited gradient-enhanced ¹H-¹³C heteronuclear single quantum correlation (HSQC) NMR spectrum comprising one or more of signals 1-11 of Table 5, wherein the spectrum is generated using a sample of the oligosaccharide composition having less than 2% monomer.

TABLE 5
HSQC NMR data

| | Center Position (ppm) | | Area under the curve (AUC) |
|---|---|---|---|
| Signal | ¹H | ¹³C | (% of total areas of signals 1-11) |
| 1 | 5.04 | 108.38 | 2.05-7.82 |
| 2 | 5.02 | 101.68 | 0.34-2.01 |
| 3 | 4.98 | 98.99 | 7.28-25.71 |
| 4 | 4.81 | 101.2 | 0.29-2.32 |
| 5 | 4.6 | 104.96 | 0.23-3.87 |
| 6 | 4.45 | 103.84 | 6.52-15.87 |
| 7 | 4.19 | 82.1 | 3.61-5.43 |
| 8 | 4.14 | 76.91 | 6.03-15.36 |
| 9 | 4.11 | 81.72 | 11.13-20.24 |
| 10 | 4.06 | 77.25 | 13.96-28.58 |
| 11 | 3.98 | 83.52 | 7.93-12.69 |

In some embodiments, the oligosaccharide composition comprises at least one of signals 1-11 of the oligosaccharide composition is defined as follows in Table 6:

TABLE 6
HSQC NMR data

| | Center Position (ppm) | | Area under the curve (AUC) |
|---|---|---|---|
| Signal | ¹H | ¹³C | (% of total areas of signals 1-11) |
| 1 | 5.04 | 108.38 | 3.20-6.67 |
| 2 | 5.02 | 101.68 | 0.68-1.68 |
| 3 | 4.98 | 98.99 | 10.97-22.02 |
| 4 | 4.81 | 101.2 | 0.70-1.92 |
| 5 | 4.6 | 104.96 | 0.96-3.14 |
| 6 | 4.45 | 103.84 | 8.39-14.00 |
| 7 | 4.19 | 82.1 | 3.97-5.07 |
| 8 | 4.14 | 76.91 | 7.89-13.50 |
| 9 | 4.11 | 81.72 | 12.95-18.42 |
| 10 | 4.06 | 77.25 | 16.88-25.66 |
| 11 | 3.98 | 83.52 | 8.88-11.74 |

HSQC NMR spectra that characterize individual batches of the selected oligosaccharide composition are described in Examples 8 and 15. A composite of the data described in Examples 8 and 15 provide an average HSQC NMR spectra defined as follows in Table 7:

TABLE 7
HSQC NMR data (composite data)

| | | | Mean +5 std | Mean +3 std | | Mean −3 std | Mean −5 std |
|---|---|---|---|---|---|---|---|
| | | | dev. AUC | dev. AUC | Mean AUC | dev. AUC | dev. AUC |
| | Center | | (% of total | (% of total | (% of total | (% of total | (% of total |
| | Position (ppm) | | areas of | areas of | areas of | areas of | areas of |
| Signal | ¹H | ¹³C | signals 1-11) | signals 1-11) | signals 1-11) | signals 1-11) | signals 1-11) |
| 1 | 5.04 | 108.38 | 7.82 | 6.67 | 4.93 | 3.20 | 2.05 |
| 2 | 5.02 | 101.68 | 2.01 | 1.68 | 1.18 | 0.68 | 0.34 |
| 3 | 4.98 | 98.99 | 25.71 | 22.02 | 16.50 | 10.97 | 7.28 |
| 4 | 4.81 | 101.2 | 2.32 | 1.92 | 1.31 | 0.70 | 0.29 |
| 5 | 4.6 | 104.96 | 3.87 | 3.14 | 2.05 | 0.96 | 0.23 |
| 6 | 4.45 | 103.84 | 15.87 | 14.00 | 11.20 | 8.39 | 6.52 |
| 7 | 4.19 | 82.1 | 5.43 | 5.07 | 4.52 | 3.97 | 3.61 |
| 8 | 4.14 | 76.91 | 15.36 | 13.50 | 10.70 | 7.89 | 6.03 |
| 9 | 4.11 | 81.72 | 20.24 | 18.42 | 15.68 | 12.95 | 11.13 |
| 10 | 4.06 | 77.25 | 28.58 | 25.66 | 21.27 | 16.88 | 13.96 |
| 11 | 3.98 | 83.52 | 12.69 | 11.74 | 10.31 | 8.88 | 7.93 |

As used herein, the term “heteronuclear single quantum correlation (HSQC) NMR” can be used interchangeably with the term “heteronuclear single quantum coherence (HSQC) NMR.”

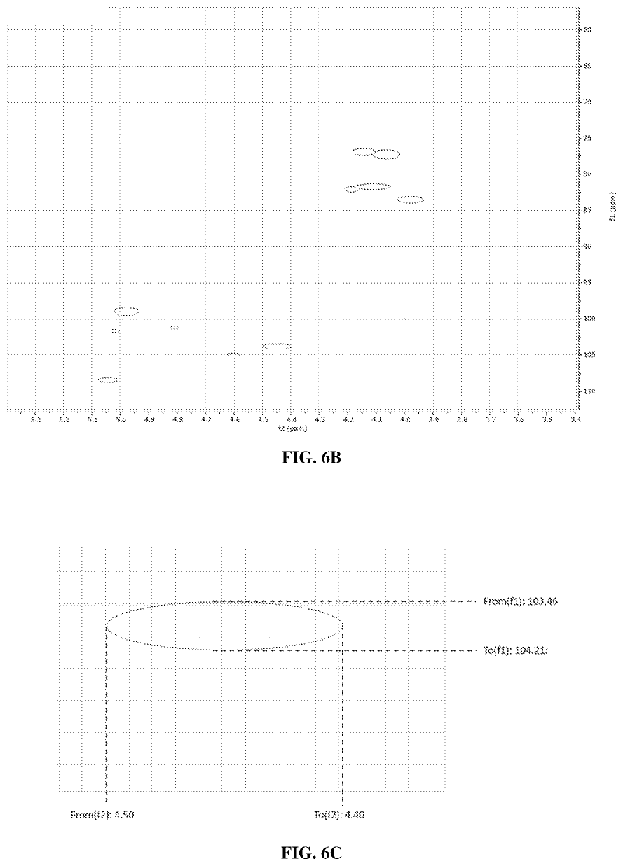

As used herein, the term “area under the curve” or “AUC” refers to the relative size (i.e., relative intensity, relative volume) of peaks/signals in an NMR spectrum (e.g., relative size of signals 1-11 of an HSQC NMR spectrum of the selected oligosaccharide composition). The AUC or relative size of peaks/signals defined herein represents the integration of integral regions using an elliptical shape. The elliptical shape can be defined by major axis coordinates and minor axis coordinates. An example of an elliptical shape defined by major axis coordinates (F2 dimension; 1H) and minor axis coordinates (F1 dimension; ¹³C) is shown in FIG. 6C. Thus, an AUC can then be determined by integrating within the confines of that elliptical shape to obtain the volume above or below the ellipse.

In some embodiments, oligosaccharide compositions having a plurality of oligosaccharides that are being characterized by a multiplicity-edited gradient-enhanced ¹H-¹³C heteronuclear single quantum correlation (HSQC) NMR spectrum comprises one or more of signals 1-11 having an ¹H integral region and a ¹³C integral region, defined as follows in Table 8:

TABLE 8
Coordinates of HSQC NMR integral regions

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | To | Position | From | to |
| 1 | 5.04 | 5.08 | 5.01 | 108.38 | 108.06 | 108.70 |
| 2 | 5.02 | 5.04 | 5.00 | 101.68 | 101.47 | 101.90 |
| 3 | 4.98 | 5.02 | 4.94 | 98.99 | 98.40 | 99.58 |
| 4 | 4.81 | 4.82 | 4.79 | 101.2 | 100.99 | 101.42 |
| 5 | 4.6 | 4.62 | 4.58 | 104.96 | 104.74 | 105.17 |
| 6 | 4.45 | 4.50 | 4.40 | 103.84 | 103.46 | 104.21 |
| 7 | 4.19 | 4.21 | 4.16 | 82.1 | 81.72 | 82.48 |
| 8 | 4.14 | 4.19 | 4.10 | 76.91 | 76.43 | 77.39 |
| 9 | 4.11 | 4.17 | 4.05 | 81.72 | 81.35 | 82.10 |
| 10 | 4.06 | 4.11 | 4.02 | 77.25 | 76.61 | 77.90 |
| 11 | 3.98 | 4.03 | 3.93 | 83.52 | 83.09 | 83.95 |

In some embodiments, an NMR spectrum is obtained by subjecting a sample of the oligosaccharide composition to a multiplicity-edited gradient-enhanced ¹H-¹³C heteronuclear single quantum coherence (HSQC) experiment using an echo-antiecho scheme for coherence selection using the following pulse sequence diagram, acquisition parameters and processing parameters:

In certain embodiments, the NMR spectrum is obtained by subjecting a sample of the composition to HSQC NMR, wherein the sample is a solution in a deuterated solvent. Suitable deuterated solvents in include deuterated acetonitrile, deuterated acetone, deuterated methanol, D₂O, and mixtures thereof. In a particular embodiment, the deuterated solvent is D₂O. Further, in some embodiments, an oligosaccharide composition being characterized by HSQC NMR has been subjected to a de-monomerization procedure such that the oligosaccharide composition comprises less than 10% monomer (e.g., less than 8%, 6%, 5%, 4%, 2%, or 1% monomer).

In certain embodiments, the NMR spectrum is obtained using the conditions described in Example 8.

Exemplary oligosaccharide compositions may be prepared according to the procedures described herein.

### III. Methods of Use

Provided herein are methods to decrease inflammation in a subject in need thereof. In some embodiments, methods include administering the selected oligosaccharide compositions described herein to a subject (e.g., to the gastrointestinal tract of a subject) to decrease inflammation in the subject. In some embodiments, oligosaccharide compositions described herein may be used to decrease inflammation in a subject having or suspected of having an inflammatory and immune disorder (e.g., an autoimmune and allergic disorder). In some embodiments, oligosaccharide compositions described herein may be used to decrease inflammation in a subject having or suspected of having a chronic inflammation disorder (e.g., an inflammatory bowel disease, e.g., UC or CD). In some embodiments, oligosaccharide compositions described herein may be used to decrease local inflammation (e.g., intestinal inflammation) in a subject (e.g., a subject having or suspected of having a chronic inflammation disorder such as ulcerative colitis (UC)). In some embodiments, oligosaccharide compositions described herein may be used to decrease local inflammation (e.g., intestinal inflammation) in a subject, e.g., the severity of local inflammation and/or the size of the inflamed local area (e.g., intestinal area of inflammation). In some embodiments, oligosaccharide compositions described herein may be used to decrease inflammation in a subject having or suspected of having ulcerative colitis (UC), Crohn's disease (CD), granulomatous colitis, indeterminate colitis, diversion colitis, pouchitis, Behcet's disease, microscopic colitis, diverticulosis-associated colitis, collagenous colitis, lymphocytic colitis, and pediatric-onset inflammatory bowel disease.

Treatment of an inflammation disorder such as ulcerative colitis (UC) can be assessed by determining levels of biomarkers of inflammation in stool and/or blood. In some embodiments, inflammation is assessed locally (e.g., local intestinal inflammation). In some embodiments, inflammation is assessed systemically (e.g., whole body inflammation). In some embodiments, administration of the selected oligosaccharide compositions described herein to a human subject result in modulation of one or more biomarkers of inflammation (e.g., in stool/fecal samples). Examples of biomarkers of inflammation that are modulated by the selected oligosaccharide composition include calprotectin (e.g., fecal calprotectin), lactoferrin (e.g., fecal lactoferrin), and lipocalin (e.g., fecal lipocalin). Additional examples of biomarkers of inflammation that may be modulated by the selected oligosaccharide composition include high-sensitivity C-reactive protein (hsCRP), LPS-binding protein (LBP), intestinal fatty acid-binding protein (I-FABP), TNFα, IL-1β, IL-6, IL-12, IFNγ, IL-2, IL-4, IL-13, IL-8, and IL-10.

Calprotectin is a protein biomarker which is found in cells involved in the immune responses to pathogens, such as neutrophil, monocytes, and macrophages (Gaya et al, 2002, QJM: An International Journal of Medicine, Volume 95, Issue 9, September 2002, Pages 557-558; Roseth et al, 2004, Scandinavian Journal of Gastroenterology, 39:10, 1017-1020). It can account for as much as 60% of the cytoplasmic proteins in neutrophils. During intestinal inflammation, neutrophils migrate through the intestinal epithelium into the intestinal lumen, leading to increased quantities of calprotectin in the stool (Masoodi et al, 2011, Ger Med Sci). The level of fecal calprotectin correlates with the number of neutrophils in the intestinal lumen and is elevated in inflammatory bowel diseases (IBD), such as Crohn's disease and ulcerative colitis (Konikoff M. R., Inflamm Bowel Dis. 2006 June; 12 (6): 524-34.).

In some embodiments, administration of oligosaccharide composition, e.g., an effective dose of oligosaccharide composition, to a subject causes a decrease in levels of fecal calprotectin in a stool/fecal sample belonging to the subject by at least 1%, 2%, 3%, 4%, 5%, 6%, 7%, 8%, 9%, 10%, 20%, 30%, 40%, 50%, 60%, 70%, 80%, 90%, or 100%, relative to a reference measurement (e.g., a measurement obtained prior to administration of the composition). In some embodiments, administration of an oligosaccharide composition to a subject causes a decrease in levels of fecal calprotectin in a stool/fecal sample belonging to the subject by 1-10%, 5-20%, 10-25%, 20-40%, 30-50%, 40-60%, 50-70%, 60-80%, 70-90%, 80-100%, or 90-100%, relative to a reference measurement (e.g., a measurement obtained prior to administration of the composition). In some embodiments, administration of an oligosaccharide composition to a subject causes a decrease in levels of fecal calprotectin in a stool/fecal sample belonging to the subject within about 1 week, 2 weeks, 4 weeks, 6 weeks, 8 weeks, 10 weeks, 12 weeks, 26 weeks, 52 weeks of treatment (e.g., after first administration, e.g., of an effective dose of the selected oligosaccharide to the subject).

In some embodiments, levels of fecal calprotectin in a stool/fecal sample belonging to the subject are less than 200 μg/g, 150 μg/g, 100 μg/g, 75 μg/g, or 50 μg/g following administration of an oligosaccharide composition to a subject.

In some embodiments, administration of oligosaccharide composition, e.g., an effective dose of oligosaccharide composition, to a subject causes a decrease in levels of fecal lactoferrin in a stool/fecal sample belonging to the subject by at least 1%, 2%, 3%, 4%, 5%, 6%, 7%, 8%, 9%, 10%, 20%, 30%, 40%, 50%, 60%, 70%, 80%, 90%, or 100%, relative to a reference measurement (e.g., a measurement obtained prior to administration of the composition). In some embodiments, administration of an oligosaccharide composition to a subject causes a decrease in levels of fecal lactoferrin in a stool/fecal sample belonging to the subject by 1-10%, 5-20%, 10-25%, 20-40%, 30-50%, 40-60%, 50-70%, 60-80%, 70-90%, 80-100%, or 90-100%, relative to a reference measurement (e.g., a measurement obtained prior to administration of the composition). In some embodiments, administration of an oligosaccharide composition to a subject causes a decrease in levels of fecal lactoferrin in a stool/fecal sample belonging to the subject within about 1 week, 2 weeks, 4 weeks, 6 weeks, 8 weeks, 10 weeks, 12 weeks, 26 weeks, 52 weeks of treatment (e.g., after first administration, e.g., of an effective dose of the selected oligosaccharide to the subject).

In some embodiments, levels of fecal lactoferrin in a stool/fecal sample belonging to the subject are less than 20 μg/g, 15 μg/g, 10 μg/g, 5 μg/g, or 3 μg/g following administration of an oligosaccharide composition to a subject.

In some embodiments, administration of oligosaccharide composition, e.g., an effective dose of oligosaccharide composition, to a subject causes a decrease in levels of fecal lipocalin in a stool/fecal sample belonging to the subject by at least 1%, 2%, 3%, 4%, 5%, 6%, 7%, 8%, 9%, 10%, 20%, 30%, 40%, 50%, 60%, 70%, 80%, 90%, or 100%, relative to a reference measurement (e.g., a measurement obtained prior to administration of the composition). In some embodiments, administration of an oligosaccharide composition to a subject causes a decrease in levels of fecal lipocalin in a stool/fecal sample belonging to the subject by 1-10%, 5-20%, 10-25%, 20-40%, 30-50%, 40-60%, 50-70%, 60-80%, 70-90%, 80-100%, or 90-100%, relative to a reference measurement (e.g., a measurement obtained prior to administration of the composition). In some embodiments, administration of an oligosaccharide composition to a subject causes a decrease in levels of fecal lipocalin in a stool/fecal sample belonging to the subject within about 1 week, 2 weeks, 4 weeks, 6 weeks, 8 weeks, 10 weeks, 12 weeks, 26 weeks, 52 weeks of treatment (e.g., after first administration, e.g., of an effective dose of the selected oligosaccharide to the subject).

In some embodiments, levels of fecal lipocalin in a stool/fecal sample belonging to the subject are less than 2000 ng/g, 1500 ng/g, 1000 ng/g, 500 ng/g, or 250 ng/g following administration of an oligosaccharide composition to a subject.

In some embodiments, administration of an oligosaccharide composition to a subject causes a decrease in levels of fecal calprotectin, fecal lactoferrin, and/or fecal lipocalin in a stool/fecal sample belonging to the subject, while not causing a similar decrease in plasma levels of calprotectin, lactoferrin, and/or lipocalin. This indicates, in some embodiments, that the selected oligosaccharide compositions may act locally, e.g., on local intestinal inflammation.

Oligosaccharide compositions described herein may be used to modulate, e.g., increase, levels of short chain fatty acids (SCFAs), e.g., butyrate, acetate, and propionate. In embodiments, oligosaccharide compositions described herein may be used to modulate, e.g., increase, levels of short chain fatty acids (SCFAs), e.g., butyrate, acetate, and propionate, e.g., in subjects exhibiting an inflammatory condition or disease, e.g., a chronic inflammatory condition, such as, e.g., inflammatory bowel diseases, such as UC and CD. In some embodiments, oligosaccharide compositions described herein may be used to increase levels of short chain fatty acids (SCFAs), e.g., butyrate, acetate, and propionate, in a subject. In some embodiments, oligosaccharide compositions described herein may be used to increase levels of short chain fatty acids (SCFAs), e.g., butyrate, acetate, and propionate, in the gastrointestinal tract of a subject. In some embodiments, provided herein is a method of increasing the concentration of butyrate in a subject by administering oligosaccharide compositions described herein to the subject. In some embodiments, provided herein is a method of increasing the concentration of propionate in a subject by administering oligosaccharide compositions described herein to the subject. In some embodiments, provided herein is a method of increasing the concentration of acetate in a subject by administering oligosaccharide compositions described herein to the subject.

Oligosaccharide compositions described herein may be used to modulate, e.g., decrease, the abundance of pathogenic and/or pro-inflammatory microbes relative to commensal microbes. In embodiments, oligosaccharide compositions described herein may be used to modulate, e.g., decrease, the abundance of pathogenic and/or pro-inflammatory microbes relative to commensal microbes, e.g., in subjects exhibiting an inflammatory condition or disease, e.g., a chronic inflammatory condition, such as, e.g., inflammatory bowel diseases, such as UC and CD. In some embodiments, the oligosaccharide compositions described herein may be used to promote the growth and/or abundance (e.g., relative abundance) of commensal microbes (e.g., Parabacteroides and Bacteroides). In some embodiments, the oligosaccharide compositions described herein may be used to reduce the growth and/or abundance (e.g., relative abundance) of pathiobionts and/or pro-inflammatory microbial taxa (e.g., taxa from the Enterobacteriaceae family). In some embodiments, the oligosaccharide compositions described herein may be used to increase the ratio of commensal bacteria relative to pathogenic and/or pro-inflammatory bacteria. In some embodiments, provided herein is a method of decreasing the abundance of pathogenic and/or pro-inflammatory microbes relative to commensal microbes in a subject (e.g., the gastrointestinal tract of a subject) by administering oligosaccharide compositions described herein to the subject. In some embodiments, provided herein is a method of increasing the abundance of commensal microbes relative to pathogenic and/or pro-inflammatory microbes in a subject (e.g., the gastrointestinal tract of a subject) by administering oligosaccharide compositions described herein to the subject.

In some embodiments, provided herein are methods of decreasing inflammation in a subject in need thereof by increasing the total concentrations or amounts of SCFAs (e.g., butyrate, propionate, and/or acetate) in the subject (e.g., by administering an oligosaccharide composition described herein). In some embodiments, provided herein are methods of decreasing inflammation in a subject in need thereof by decreasing the abundance of pathogenic and/or pro-inflammatory microbes relative to commensal microbes in the subject (e.g., the gastrointestinal tract of the subject) by administering an oligosaccharide composition described herein to the subject, e.g., subjects exhibiting an inflammatory condition or disease, e.g., a chronic inflammatory condition, such as, e.g., inflammatory bowel diseases, such as UC and CD.

In some embodiments, oligosaccharide compositions described herein may be used to increase levels of short chain fatty acids (SCFAs), e.g., butyrate, acetate, and propionate, in a subject having or suspected of having an autoimmune and/or allergic disorder (e.g., chronic inflammatory disorder, e.g., an inflammatory bowel diseases, e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease). In some embodiments, the selected oligosaccharide composition is administered to a subject in an amount effective to increase levels of short chain fatty acids (SCFAs), e.g., butyrate, acetate, and propionate (e.g., in the gastrointestinal tract) in a subject in need thereof.

In some embodiments, oligosaccharide compositions described herein may be used to decrease the abundance of pathogenic and/or pro-inflammatory microbes relative to commensal microbes in a subject (e.g., in the gastrointestinal tract of a subject) having or suspected of having an autoimmune and/or allergic disorder (e.g., chronic inflammatory disorder, e.g., an inflammatory bowel diseases, e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease). In some embodiments, the selected oligosaccharide composition is administered to a subject (e.g., in the gastrointestinal tract of a subject) in an amount effective to decrease the abundance of pathogenic and/or pro-inflammatory microbes relative to commensal microbes in a subject in need thereof.

In some embodiments, administration of an oligosaccharide composition to a subject is effective in treating dysbiosis and diseases or disorders associated with high relative abundance of pathogenic bacteria relative to commensal bacteria, and/or low levels of SCFAs. In some embodiments, administration of an oligosaccharide composition to a subject is effective in treating an autoimmune and/or allergic disorder (e.g., chronic inflammatory disorder, e.g., an inflammatory bowel diseases, e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease). In some embodiments, administration of an oligosaccharide composition to a subject is effective in treating ulcerative colitis. In some embodiments, administration of an oligosaccharide composition to a subject is effective in treating Crohn's disease.

In some embodiments, administration of an oligosaccharide composition to a subject having an autoimmune and/or allergic disorder (e.g., chronic inflammatory disorder) increases levels of short chain fatty acids (SCFAs), e.g., butyrate, acetate, and propionate (e.g., in the gastrointestinal tract of the subject). In some embodiments, administration of an oligosaccharide composition to a subject having an inflammatory bowel disease (e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease) increases levels of short chain fatty acids (SCFAs), e.g., butyrate, acetate, and propionate (e.g., in the gastrointestinal tract of the subject).

In some embodiments, provided is a method of modulating the microbial community composition and/or the metabolic output of the microbial community in a subject, e.g. modulating the environment, e.g., to modulate (e.g., increase) levels of short chain fatty acids (e.g., butyrate, acetate, and/or propionate). In some embodiments, an oligosaccharide composition described herein promotes the growth and/or abundance (e.g., relative abundance) of propionate-producing bacteria (e.g., belonging to the phylum Parabacteroides). In some embodiments, an oligosaccharide composition described herein promotes the growth and/or abundance (e.g., relative abundance) of butyrate-producing bacteria (e.g., Lachnospiraceae and Eubacteriaceae). In some embodiments, an oligosaccharide composition is administered in an effective amount to modulate the microbial community and alter the environment of the GI tract, (e.g., altering pH, altering lactic acid, altering microbial density, etc.).

In some embodiments, administration of an oligosaccharide composition to a subject causes an increase in levels of total short chain fatty acids in the subject (e.g., in the gastrointestinal tract of the subject) by at least 1%, 2%, 3%, 4%, 5%, 6%, 7%, 8%, 9%, 10%, 20%, 30%, 40%, 50%, 60%, 70%, 80%, 90%, 100%, 125%, 150%, or 200%, relative to a reference measurement (e.g., a measurement obtained prior to administration of the composition). In some embodiments, administration of an oligosaccharide composition to a subject causes an increase in levels of total short chain fatty acids in the subject (e.g., in the gastrointestinal tract of the subject) by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%, relative to a reference measurement (e.g., a measurement obtained prior to administration of the composition).

In some embodiments, a reference of baseline measurement of short chain fatty acids in a subject is as described in Venegas, D. P. et. al., Short Chain Fatty Acids (SCFAs)-Mediated Gut Epithelial and Immune Regulation and Its Relevance for Inflammatory Bowel Diseases., Front. Immunol., Vol. 10, Art. 277, 11 Mar. 2019. In some embodiments, a reference of baseline measurement of short chain fatty acids in a subject is as described in Cummings, J. H. et. al., Short chain fatty acids in human large intestine, portal, hepatic and venous blood., Gut, 1987, 28, 1221-1227.

In some embodiments, administration of an oligosaccharide composition to a subject causes an increase in levels of butyrate in the subject (e.g., in the gastrointestinal tract of the subject) by at least 1%, 2%, 39%, 4%, 5%, 6%, 7%, 8%, 9%, 10%, 20%, 30%, 40%, 50%, 60%, 70%, 80%, 90%, 100%, 125%, 150%, or 200%, relative to a reference measurement (e.g., a measurement obtained prior to administration of the composition). In some embodiments, administration of an oligosaccharide composition to a subject causes an increase in levels of butyrate in the subject (e.g., in the gastrointestinal tract of the subject) by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%, relative to a reference measurement.

In some embodiments, administration of an oligosaccharide composition to a subject causes an increase in levels of acetate in the subject (e.g., in the gastrointestinal tract of the subject) by at least 1%, 2%, 3%, 4%, 5%, 6%, 7%, 8%, 9%, 10%, 20%, 30%, 40%, 50%, 60%, 70%, 80%, 90%, 100%, 125%, 150%, or 200%, relative to a reference measurement (e.g., a measurement obtained prior to administration of the composition). In some embodiments, administration of an oligosaccharide composition to a subject causes an increase in levels of acetate in the subject (e.g., in the gastrointestinal tract of the subject) by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%, relative to a reference measurement.

In some embodiments, administration of an oligosaccharide composition to a subject causes an increase in levels of propionate in the subject (e.g., in the gastrointestinal tract of the subject) by at least 1%, 2%, 3%, 4%, 5%, 6%, 7%, 8%, 9%, 10%, 20%, 30%, 40%, 50%, 60%, 70%, 80%, 90%, 100%, 125%, 150%, or 200%, relative to a reference measurement (e.g., a measurement obtained prior to administration of the composition). In some embodiments, administration of an oligosaccharide composition to a subject causes an increase in levels of propionate in the subject (e.g., in the gastrointestinal tract of the subject) by 1-10%, 5-20%, 10-25%, 20-40%, 30-50%, 40-60%, 50-70%, 60-80%, 70-90%, 80-100%, 90-110%, 100-125%, 110-150%, 125-175%, or 150-200%, relative to a reference measurement.

In some embodiments, the compositions and methods described herein can be used to treat at least one symptom (e.g., one, two, three, or four or more) of an autoimmune and/or allergic disorder (e.g., chronic inflammatory disorder). In some embodiments, the compositions and methods described herein can be used to treat at least one symptom (e.g., one, two, three, or four or more) of an inflammatory bowel diseases (e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease). Symptoms of inflammatory bowel diseases include frequency of diarrhea, severity of diarrhea, abdominal pain and cramping, loose stools, bloody stools, rectal pain, rectal bleeding, urgency to defecate, inability to defecate despite urgency, weight loss, loss of appetite, fatigue, and fever.

In some embodiments, treating a subject having an autoimmune and/or allergic disorder (e.g., chronic inflammatory disorder, e.g., inflammatory bowel disease, e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease) includes combining administering the oligosaccharide composition and a standard-of-care treatment. Standard-of-care treatments include steroids, corticosteroids (e.g., prednisone and/or budesonide), immunomodulator drugs (e.g., azathioprine, mercaptopurine, cyclosporine, and/or tofacitinib), aminosalicylates (e.g., 5-aminosalicylic acid and derivative thereof, sulfasalazine (Azulfidine), mesalamine (Asacol HD, Delzicol, others), balsalazide (Colazal), and olsalazine (Dipentum)), and biologics (e.g., anti-TNF molecules such as adalimumab, Infliximab, golimumab, certolizumab pegol, anti-integrin molecules such as natalizumab, Vedolizumab, and/or IL-12 and/or IL-23 agonists such as ustekinumab). In some embodiments, treating a subject having ulcerative colitis comprises administration of the oligosaccharide composition concurrently with a standard-of-care treatment (e.g., treatment with corticosteroids, steroids, aminosalicylates, immunomodulator drugs, biologics, etc.). In some embodiments, treating a subject having Crohn's disease comprises administration of the oligosaccharide composition concurrently with a standard-of-care treatment (e.g., treatment with corticosteroids, steroids, aminosalicylates, immunomodulator drugs, biologics, etc.).

In some embodiments, a subject having autoimmune and/or allergic disorder (e.g., chronic inflammatory disorder, e.g., inflammatory bowel disease, e.g., ulcerative colitis, Crohn's disease, granulomatous colitis, indeterminate colitis, diversion colitis, pouchitis, Behcet's disease, microscopic colitis, diverticulosis-associated colitis, collagenous colitis, lymphocytic colitis, and/or pediatric-onset inflammatory bowel disease) in need of an oligosaccharide composition as described herein is a subject who has previously had surgery (e.g., to remove a damaged portion of the gastrointestinal tract) or a standard-of-care treatment intervention (e.g., treatment with corticosteroids, steroids, aminosalicylates, immunomodulator drugs, biologics, etc.) for said disorder.

The compounds and compositions provided herein may be used in methods to modulate bacterial taxa (e.g. 1, 2, 3, 4, 5 or more taxa) present in the microbiota of a subject. In some embodiments, modulation comprises a reduction in the abundance (e.g., relative abundance) of pro-inflammatory and/or pathogenic microbial taxa (e.g., taxa from the Enterobacteriaceae family). In some embodiments, modulation comprises a reduction in the abundance of pro-inflammatory and/or pathogenic microbial taxa (e.g., taxa from the Enterobacteriaceae family) relative to commensal microbial taxa (e.g., Parabacteroides and Bacteroides). In some embodiments, modulation comprises a reduction in the abundance (e.g., relative abundance) of Enterobacteriaceae and/or Ruminococcaceae. In some embodiments, modulation comprises a reduction in the abundance (e.g., relative abundance) of Enterobacteriaceae Escherichia and/or Ruminococcaceae Faecalibacterium. In some embodiments, modulation comprises an increase in the abundance (e.g., relative abundance) of commensal microbial taxa (e.g., taxa from Parabacteroides and Bacteroides). In some embodiments, modulation comprises an increase in the abundance (e.g., relative abundance) of Parabacteroides and Eisenbergiella. In some embodiments, modulation comprises an increase in the abundance of commensal microbial taxa (e.g., taxa from Parabacteroides and Bacteroides) relative to pro-inflammatory and/or pathogenic microbial taxa (e.g., taxa from the Enterobacteriaceae family). In some embodiments, modulation comprises an increase in the abundance (e.g., relative abundance) of Bacteroidaceae and/or Tannerellaceae. In some embodiments, modulation comprises a reduction in the abundance (e.g., relative abundance) of Bacteroidaceae Bacteroides, Tannerellaceae Parabacteroides, Escherichia, Klebsiella, Shigella, and/or Citrobacter. In some embodiments, modulation comprises a change in the structure of the microbiota, such as a change in the relative composition of a taxa or a change in the relative abundance of a taxa, e.g., relative to another taxa or relative to what would be observed in the absence of the modulation. In other embodiments, modulation comprises a change in a function of the microbiota, such as a change in gene expression, a change in gene copy number, overall abundance of DNA, level of a gene product (e.g., RNA or protein), or metabolic output of the microbiota, or a change in a functional pathway of the host (e.g., a change in gene expression, level of a gene product, or metabolic output of a host cell or host process). Methods of modulating microbial taxa disclosed in WO 2016/122889 and WO 2016/172657 which are hereby incorporated by reference, are suitable for use in methods described herein. In some embodiments, an oligosaccharide composition described herein promotes the growth and/or abundance (e.g., relative abundance) of propionate-producing bacteria (e.g., belonging to the phylum Parabacteroides). In some embodiments, an oligosaccharide composition described herein promotes the growth and/or abundance (e.g., relative abundance) of butyrate-producing bacteria (e.g., Lachnospiraceae and Eubacteriaceae),

The methods describe herein include administering to a subject a composition described herein, e.g., comprising an oligosaccharide composition described herein, in an amount effective to modulate taxa. In some embodiments, the abundance of a bacterial taxa may increase relative to other taxa (or relative from one point in time to another) when the composition is administered, and the increase can be at least a 5%, 10%, 25% 50%, 75%, 100%, 250%, 500%, 750% increase or at least a 1000% increase. The abundance of a bacterial taxa may also decrease relative to other taxa (or relative from one point in time to another) when the composition is administered, and the decrease can be at least a 5%, 10%, 25% 50%, 75%, 85%, 90%, 95%, 96%, 97%, 98%, 99% decrease, or at least a 99.9% decrease. Administration of the composition can modulate the abundance of the desired and/or non-desired bacterial taxa in the subject's gastrointestinal microbiota.

In some embodiments, a composition described herein, e.g., comprising an oligosaccharide composition described herein, modulates (e.g. substantially increase or substantially decrease) the growth (and the total number) of (or substantially increase or substantially decrease the relative representation/abundance in the total (gastrointestinal) community of one or more of (e.g. 1, 2, 3, 4, 5, 6, 7, 8, 9, 10, or more) bacterial taxa.

In some embodiments, a composition described herein, e.g., comprising an oligosaccharide composition described herein, substantially increases the growth, e.g. the total number or the relative representation/abundance in the total (gastrointestinal) community of one or more of (e.g. 1, 2, 3, 4, 5, 6, 7, 8, 9, 10, or more) commensal bacterial taxa. In some embodiments, a composition described herein, e.g., comprising an oligosaccharide composition described herein, substantially increases the growth, e.g. the total number or the relative representation/abundance in the total (gastrointestinal) community of Parabacteroides and Bacteroides. In some embodiments, a composition described herein, e.g., comprising an oligosaccharide composition described herein, substantially increases the growth, e.g. the total number or the relative representation/abundance in the total (gastrointestinal) community of Bacteroidaceae and/or Tannerellaceae.

In some embodiments, a composition described herein, e.g., comprising an oligosaccharide composition described herein, substantially decreases the growth, e.g. the total number or the relative representation/abundance in the total (gastrointestinal) community of one or more of (e.g. 1, 2, 3, 4, 5, 6, 7, 8, 9, 10, or more) bacterial taxa. In some embodiments, a composition described herein, e.g., comprising an oligosaccharide composition described herein, substantially decreases the growth, e.g. the total number or the relative representation/abundance in the total (gastrointestinal) community of Enterobacteriaceae and/or Ruminococcaceae. In some embodiments, a composition described herein, e.g., comprising an oligosaccharide composition described herein, substantially decreases the growth, e.g. the total number or the relative representation/abundance in the total (gastrointestinal) community of Enterobacteriaceae Escherichia and/or Ruminococcaceae Faecalibacterium.

In some embodiments, administration of a selected oligosaccharide composition to a subject (e.g., to the gut of a subject) causes a depletion (i.e., decrease in levels) of genes associated with adherent-invasive Escherichia coli within the microbiome of the gastrointestinal tract of the subject (e.g., as measured in a fecal sample of the subject). Assessment of the levels (e.g., metagenomic assessment) represents a mechanism for assessing the concentration and abundance of adherent-invasive Escherichia coli. In some embodiments, assessment of the levels of genes associated with adherent-invasive Escherichia coli is performed using Pangenome-based Phylogenomic Analysis (PanPhlAn) (e.g., of metagenomic data) used to quantify the pangenome of E. coli genes (e.g., using a pangenome reference of 90% amino acid identity gene clusters of such genes). These bacterial taxa have been implicated in the pathogenesis of inflammatory bowel diseases such as ulcerative colitis. See, e.g., Palmela C., et al. “Adherent-invasive Escherichia coli in inflammatory bowel disease,” Gut 2018; 67:574-587. Genes associated with adherent-invasive E. coli may include fimH, ompA, ompC, fim operon/fimH, chiA, nlpI, yfgL, ibeA, afaC, vat-AIEC, fliC, vgrG, hcp, vasD, vasG, impL, impK, and others known to one skilled in the art. In some embodiments, administration of an oligosaccharide composition to a subject causes a depletion (i.e., decrease in levels) of genes associated with adherent-invasive Escherichia coli (e.g., fimH, ompA, ompC) within the microbiome of the gastrointestinal tract of the subject by at least 1%, 2%, 3%, 4%, 5%, 6%, 7%, 8%, 9%, 10%, 20%, 30%, 40%, 50%, 60%, 70%, 80%, 90%, or 100%, relative to a reference measurement (e.g., a measurement obtained prior to administration of the composition, e.g., measured in a fecal sample of the subject.). In some embodiments, administration of an oligosaccharide composition to a subject causes a depletion (i.e., decrease in levels) of genes associated with adherent-invasive Escherichia coli (e.g., fimH, ompA, ompC) within the microbiome of the gastrointestinal tract of the subject by 1-10%, 5-20%, 10-25%, 20-40%, 30-50%, 40-60%, 50-70%, 60-80%, 70-90%, 80-100%, 90-100%, relative to a reference measurement (e.g., a measurement obtained prior to administration of the composition, e.g., measured in a fecal sample of the subject).

In some embodiments, an oligosaccharide composition described herein modulates the growth e.g. the total number or the relative representation/abundance in the total (gastrointestinal) community of bacterial taxa and species that consume cholesterol, e.g., at higher rates than a reference bacterial taxa or species.

In some embodiments, the oligosaccharide composition is formulated as powder, e.g., for reconstitution (e.g., in water) for oral administration. In some embodiments, the oligosaccharide composition is formulated in a solid form (e.g., chewable tablet or gummy) for oral administration. In some embodiments, the oligosaccharide composition is formulated as a pharmaceutical composition for delivery by a feeding tube. In some embodiments, the oligosaccharide composition is formulated as a pharmaceutical composition for delivery by total parenteral nutrition (TPN).

The oligosaccharide composition may be administered to the subject on a daily, weekly, biweekly, or monthly basis. In some embodiments, the composition is administered to the subject daily. In some embodiments, the composition is administered to the subject more than once per day (e.g., 2, 3, or 4 times per day). In some embodiments, the composition is administered to the subject more than once per week (e.g., 2, 3, or 4 times per week). In some embodiments, the composition is administered to the subject once or twice per day for one, two, three, four, five, six, seven, eight, nine, or ten weeks in a row. In some embodiments, the composition is administered to the subject chronically. In some embodiments, the composition is administered to the subject for 2, 3, 4, 5, 6, 7, 8, 9, 10, 11, 12, or more continuous months. In some embodiments, a higher dose is administered initially, e.g., for 1-2 weeks, 1-4 weeks, 1-6 weeks, 1-8 weeks, 1-10 weeks, 1-12 weeks, and the dose is lowered (e.g., by 20%, 30%, 40%, 50%, 60%, 70%, 80% or 90%), e.g., for chronic or long-term administration to the subject.

In some embodiments, the oligosaccharide composition may be administered to a subject having a chronic disorder (e.g., inflammatory bowel disease) on a daily or weekly basis. In some embodiments, the oligosaccharide composition may be administered to a subject having a chronic disorder (e.g., inflammatory bowel disease) twice a day in an amount effective to treat the disorder. In some embodiments, the oligosaccharide composition may be administered to a subject having a chronic disorder (e.g., inflammatory bowel disease) twice a day at maximum tolerated dose.

In some embodiments, an effective amount of an oligosaccharide composition is a total of 5-200 grams, 5-150 grams, 5-100 grams, 5-75 grams, 5-50 grams, 5-25 grams, 10-50 grams, 25-50 grams, 30-60 grams, 50-75 grams, 50-100 grams, or 40-80 grams administered daily.

The oligosaccharide composition of the disclosure is well tolerated by a subject (e.g., oligosaccharide compositions do not cause or cause minimal discomfort, e.g., production of gas or gastrointestinal discomfort, in subjects). In some embodiments, 5-200 grams, 5-150 grams, 5-100 grams, 5-75 grams, 5-50 grams, 5-25 grams, 10-50 grams, 25-50 grams, 30-60 grams, 50-75 grams, 50-100 grams, or 40-80 grams of total daily dose are well tolerated by a subject. In some embodiments, a maximum tolerated dose of the oligosaccharide composition is 5-200 grams, 5-150 grams, 5-100 grams, 5-75 grams, 5-50 grams, 5-25 grams, 10-50 grams, 25-50 grams, 30-60 grams, 50-75 grams, 50-100 grams, 40-80, or more grams administered daily. Any dosage amount of an oligosaccharide composition as described herein that is administered to the subject at a single time or in a single dose may be well tolerated by the subject.

In some embodiments, the amount of the oligosaccharide composition that is administered to the subject at a single time or in a single dose is more tolerated by the subject than a similar amount of commercial low-digestible sugars such as fructooligosaccharides (FOS). Commercial low-digestible sugars are known in the art to be poorly tolerated in subjects (See, e.g., Grabitske, H. A., Critical Reviews in Food Science and Nutrition, 49:327-360 (2009)), e.g., at high doses. For example, tolerability studies of FOS indicate that 20 grams FOS per day causes mild gastrointestinal symptoms and that 30 grams FOS per day causes major discomfort and gastrointestinal symptoms.

In some embodiments, an oligosaccharide composition described herein is co-administered with commensal or probiotic bacterial taxa and bacteria that are generally recognized as safe (GRAS) or known commensal or probiotic microbes. In some embodiments, probiotic or commensal bacterial taxa (or preparations thereof) may be administered to a subject before or after administration of an oligosaccharide composition to the subject. In some embodiments, probiotic or commensal bacterial taxa (or preparations thereof) may be administered to a subject simultaneously with administration of an oligosaccharide composition to the subject.

A commensal or probiotic bacteria is also referred to a probiotic. Probiotics can include the metabolites generated by the probiotic bacteria during fermentation. These metabolites may be released to the medium of fermentation, e.g., into a host organism (e.g., subject), or they may be stored within the bacteria. Probiotic bacteria includes bacteria, bacterial homogenates, bacterial proteins, bacterial extracts, bacterial ferment supernatants and combinations thereof, which perform beneficial functions to the host animal, e.g., when given at a therapeutic dose.

Useful probiotics include at least one lactic acid and/or acetic acid and/or propionic acid producing bacteria, e.g., microbes that produce lactic acid and/or acetic acid and/or propionic acid by decomposing carbohydrates such as glucose and lactose. Preferably, the probiotic bacteria is a lactic acid bacterium. In embodiments, lactic acid bacteria include Lactobacillus, Leuconostoc, Pediococcus, Streptococcus, and Bifidobacterium. Suitable probiotic bacteria can also include other bacterias which beneficially affect a host by improving the hosts intestinal microbial balance, such as, but not limited to yeasts such as Saccharomyces, Debaromyces, Candida, Pichia and Torulopsis, molds such as Aspergillus, Rhizopus, Mucor, and Penicillium and Torulopsis, and other bacteria such as but not limited to the genera Bacteroides, Clostridium, Fusobacterium, Melissococcus, Propionibacterium, Enterococcus, Lactococcus, Staphylococcus, Peptostreptococcus, Bacillus, Pediococcus, Micrococcus, Leuconostoc, Weissella, Aerococcus, and Oenococcus, and combinations thereof.

Non-limiting examples of lactic acid bacteria useful in the disclosure herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Lactobacillus paracasei, Lactobacillus brevis, Bifidobacterium longum, Bifidobacterium infantis, Bifidobacterium bifidum, Bifidobacterium animalis, Bifidobacterium lactis, Bifidobaccterium breve, Bifidobacterium adolescentis, and Pediococcus cerevisiae and combinations thereof, in particular Lactobacillus, Bifidobacterium, and combinations thereof.

Commensal or probiotic bacteria which are particularly useful in the present disclosure include those which (for human administration) are of human origin (or of the origin of the mammal to which the probiotic bacteria is being administered), are non-pathogenic to the host, resist technological processes (i.e. can remain viable and active during processing and in delivery vehicles), are resistant to gastric acidity and bile toxicity, adhere to gut epithelial tissue, have the ability to colonize the gastrointestinal tract, produce antimicrobial substances, modulate immune response in the host, and influence metabolic activity (e.g. cholesterol assimilation, lactase activity, vitamin production).

The commensal or probiotic bacteria can be used as a single strain or a combination of multiple strains, wherein the total number of bacteria in a dose of probiotic bacteria is from about 1×10³ to about 1×10¹⁴, or from about 1×10 to about 1×10¹², or from about 1×10⁷ to about 1×10¹¹ CFU per dose.

The commensal or probiotic bacteria can be formulated with the oligosaccharide compositions while the probiotic bacteria are alive but in a state of “suspended animation” or somnolence. Once freeze-dried, the viable cultures(s) of probiotic bacteria are handled so as to minimize exposure to moisture that would reanimate the cultures because, once reanimated, the cultures can experience high rates of morbidity unless soon cultured in a high moisture environment or medium. Additionally, the cultures are handled to reduce possible exposure to high temperatures (particularly in the presence of moisture) to reduce morbidity.

The probiotic bacterias can be used in a powdered, dry form. The probiotic bacterias can also be administered in the oligosaccharide composition or in a separate oligosaccharide composition, administered at the same time or different time as the oligosaccharide compositions.

Other probiotic bacteria suitable include Bifidobacterium lactis, B. animalis, B. bifidum, B. longum, B. adolescentis, and B. infantis,

In embodiments, a commensal bacterial taxa that can be used in and/or in combination with an oligosaccharide composition described herein comprises Akkermansia, Anaerococcus, Bacteroides, Bifidobacterium (including Bifidobacterium lactis, B. animalis, B. bifidum, B. longum, B. adolescentis, B. breve, and B. infantis), Blautia, Clostridium, Corynebacterium, Dialister, Eubacterium, Faecalibacterium, Finegoldia, Fusobacterium, Lactobacillus (including, L. acidophilus, L. helveticus, L. bifidus, L. lactis, L. fermentii, L. salivarius, L. paracasei, L. brevis, L. delbruekii, L. thermophiles, L. crispatus, L. casei, L. rhamnosus, L. reuteri, L. fermentum, L. plantarum, L. sporogenes, and L. bulgaricus), Peptococcus, Peptostreptococcus, Peptoniphilus, Prevotella, Roseburia, Ruminococcus, Staphylococcus, and/or Streptococcus (including S. lactis, S. cremoris, S. diacetylactis, S. thermophiles).

In embodiments, a commensal bacterial taxa, e.g., GRAS strain, that can be used in and/or in combination with an oligosaccharide composition described herein comprises Bacillus coagulans GBI-30, 6086; Bifidobacterium animalis subsp. Lactis BB-12; Bifidobacterium breve Yakult; Bifidobacterium infantis 35624; Bifidobacterium animalis subsp. Lactis UNO 19 (DR 10); Bifidobacterium longum BB536; Escherichia coli M-17; Escherichia coli Nissle 1917; Lactobacillus acidophilus DDS-1; Lactobacillus acidophilus LA-5; Lactobacillus acidophilus NCFM; Lactobacillus casei DN 114-001 (Lactobacillus casei Immunitas(s)/Defensis); Lactobacillus casei CRLA31; Lactobacillus casei F19; Lactobacillus paracasei Stl 1 (or NCC2461); Lactobacillus johnsonii Lai (Lactobacillus LCI, Lactobacillus johnsonii NCC533); Lactococcus lactis LIA; Lactobacillus plantarum 299V; Lactobacillus reuteri ATTC 55730 (Lactobacillus reuteri SD2112); Lactobacillus rhamnosus ATCC 53013; Lactobacillus rhamnosus LB21; Saccharomyces cerevisiae (boulardii) lyo; mixture of Lactobacillus rhamnosus GR-1 and Lactobacillus reuteri RC-14; mixture of Lactobacillus acidophilus NCFM and Bifidobacterium lactis BB-12 or BL-04; mixture of Lactobacillus acidophilus CL1285 and Lactobacillus casei; and a mixture of Lactobacillus helveticus R0052, Lactobacillus rhamnosus R0011, and/or Lactobacillus rhamnosus GG (LGG).

### IV. Kits

Kits also are contemplated. For example, a kit can comprise unit dosage forms of the oligosaccharide composition, and a package insert containing instructions for use of the composition in treatment. In some embodiments, the composition is provided in a dry powder format. In some embodiments, the composition is provided in solution, powder or tablet. The kits include an oligosaccharide composition in suitable packaging for use by a subject in need thereof. Any of the compositions described herein can be packaged in the form of a kit. A kit can contain an amount of an oligosaccharide composition sufficient for an entire course of treatment, or for a portion of a course of treatment. Doses of an oligosaccharide composition can be individually packaged, or the oligosaccharide composition can be provided in bulk, or combinations thereof. Thus, in one embodiment, a kit provides, in suitable packaging, individual doses of an oligosaccharide composition that correspond to dosing points in a treatment regimen, wherein the doses are packaged in one or more packets.

Kits can further include written materials, such as instructions, expected results, testimonials, explanations, warnings, clinical data, information for health professionals, and the like. In one embodiment, the kits contain a label or other information indicating that the kit is only for use under the direction of a health professional. The container can further include scoops, syringes, bottles, cups, applicators or other measuring or serving devices.

### EXAMPLES

### Example 1. Production of Short Chain Fatty Acids (SCFA) in Healthy Stool Samples in the Presence of Oligosaccharide Compositions

Several hundred different synthetic oligosaccharide compositions (431 total compositions) were tested for their ability to modulate (e.g., reduce) the levels of metabolites (e.g., short-chain fatty acids such as acetate, propionate, and butyrate) in healthy fecal stool samples.

Fecal samples were collected from donors, frozen and stored at −80° C. until use. Frozen fecal samples were transferred to an anaerobic chamber, allowed to thaw and homogenized to a final concentration of 20% solids in phosphate buffer saline (PBS) supplemented with 15% glycerol. The 20% fecal slurries were then filtered to remove large debris, aliquoted, removed from the anaerobic chamber and immediately frozen on dry ice before storage at −80° C. On the day of the experiment, one 1 mL aliquot of each 20% fecal slurry was thawed and diluted into bacterial growth media (Clostridium minimal medium supplemented with 0.1% w/v trypticase peptone and 0.75 mM urea) to a final concentration of 1% in an anaerobic chamber. The 1% fecal slurry solutions were then dispensed into the wells of 96 deep well plates containing sterile water (negative control) or preparations of the oligosaccharide preparations (final concentration of 0.5%). Three replicates of each sample were prepared. Fecal microbial cultures were incubated anaerobically at 37° C. for 45 hours. Initial ex vivo screening was performed with three different fecal microbial communities from healthy subjects.

After the 45-hour incubation period, the 96-well deep well plates containing the fecal microbial cultures are removed from the anaerobic chamber and kept on ice. The plates were sedimented by centrifugation (3,000×g) for 10 minutes at 4° C. Fecal microbiota culture supernatants and pellets were collected and stored at −80° C. Supernatant samples were thawed and analyzed by gas chromatography with a flame ionization detector (GC-FID) to quantify the concentration of short-chain fatty acids (acetate, propionate, and butyrate) produced in the fecal samples.

Normalized SCFA concentrations were calculated by subtracting the value of each SCFA with the negative control from the value with an oligosaccharide composition. This calculation was performed to determine the extent to which the selected oligosaccharide composition increased butyrate production under these assay conditions.

One of the several hundred tested oligosaccharide compositions, the selected oligosaccharide composition as described throughout this document (e.g., as produced by the processes in Examples 2-4, 12 and 13), was determined to significantly increase butyrate production (median increase of 4.2 mM butyrate across the three tested fecal communities relative to negative control) (FIG. 19).

### Example 2. Production of Oligosaccharide Composition at 100 g Scale from Galactose Using a Soluble Acid Catalyst

A procedure was developed for the synthesis of a selected oligosaccharide composition as described in Example 1 at a 100 gram scale. 100 g of galactose and an amount of water sufficient to achieve a starting concentration of 85% dissolved solids were added to a reaction vessel (1 L three-neck round-bottom flask). The reaction vessel was equipped with a heating mantle configured with an overhead stirrer. A probe thermocouple was disposed in the vessel through a septum, such that the probe tip sat above the stir blade and not in contact with the walls of the reaction vessel. Prior to addition of catalyst, the reaction vessel was equipped with a condenser in a reflux position.

The procedure used citric acid (1.5-3% w/w) as a catalyst and de-ionized water for quenching. Following addition of catalyst, the reaction vessel was equipped in a distillation position to remove excess water throughout the course of the reaction.

The temperature controller was set to a target temperature (130 to 140° C.), and stirring of the contents of the vessel was initiated to promote uniform heat transfer and melting of the sugar solids, as the temperature of the syrup was brought to the target temperature, under ambient (atmospheric) pressure.

Upon addition of the catalyst, the reaction was maintained at the target temperature under continuous mixing for about 2.5-5 hours, determined by following the reaction by HPLC. Next, the heat was turned off while maintaining constant stirring.

The reaction was then quenched by slowly adding approximately 60 mL of deionized (DI) water (room temperature) to dilute and cool the product mixture, to target a final concentration of 50-60 wt % dissolved solids. In some embodiments of this Example, the reaction was quenched by slowly adding 60-100 mL of deionized (DI) water (room temperature) to dilute and cool the product mixture, to target a final concentration of 45-65 wt % dissolved solids. Generally, the water addition rate was performed to control the mixture viscosity as the oligosaccharide composition was cooled and diluted.

### Example 3. Production of a Selected Oligosaccharide Composition at 10 kg Scale from Galactose Using a Soluble Acid Catalyst

A procedure was developed for the synthesis of a selected oligosaccharide composition as described in Example 1 at a 10 kilogram scale. 9.1 kg of anhydrous galactose, 0.27 kg citric acid anhydrous acid catalyst and 1.45 kg water were added to a reaction vessel (22 L Littleford-Day horizontal plow mixer). A distillation condenser unit was attached to the reactor. The contents were agitated at approximately 30 RPM and the vessel temperature was gradually increased over a 3.5-4.0 hour period to about 136° C. at atmospheric pressure. The mixture was maintained at temperature for 1-1.5 hours, after which the heating was stopped and pre-heated water was gradually added to the reaction mixture at a rate of 60 mL/min until the temperature of the reactor contents decreased to 120° C., then at 150 mL/min until the temperature of the reactor contents decreased to 110° C., then at 480 mL/min until a total of 7.5 kg of water was added, and the temperature of the reactor contents decreased below 100° C. An additional 1.6 kg water was added to the reactor for further dilution. The reaction mixture was drained from the vessel, resulting in 17.0-17.6 kg of crude oligosaccharide as an aqueous solution (approximately 49-52 wt %).

The oligosaccharide composition was purified by flowing through a cationic exchange resin (Dowex® Monosphere 88H) column, two columns of decolorizing polymer resin (Dowex® OptiPore SD-2), and an anionic exchange resin (Dowex® Monosphere 77WBA) column. The resulting purified material with concentration of about 43 wt % was then concentrated to a final concentration of about 70 wt % solids by vacuum rotary evaporation to yield the purified oligosaccharide composition.

### Example 4. Production of Oligosaccharide Composition at 100 g Scale from Galactose Using a Solid Acid Catalyst

A procedure was developed for the synthesis of a selected oligosaccharide composition as described in Example 1 at a 100 gram scale. 100 g of galactose and an amount of water sufficient to achieve a starting concentration of 85% dissolved solids were added to a reaction vessel (1 L three-neck round-bottom flask). The reaction vessel was equipped with a heating mantle configured with an overhead stirrer. A probe thermocouple was disposed in the vessel through a septum, such that the probe tip sat above the stir blade and not in contact with the walls of the reaction vessel. Prior to addition of catalyst, the reaction vessel was equipped with a condenser in a reflux position.

The procedure used wetted (45-55% moisture content) Dowex Marathon-C, H+ form, (1-5% w/w, dry basis) as a catalyst and de-ionized water for quenching. Following addition of catalyst, the reaction vessel was equipped in a distillation position to remove excess water throughout the course of the reaction.

The temperature controller was set to a target temperature (130 to 145° C.), and stirring of the contents of the vessel was initiated to promote uniform heat transfer and melting of the sugar solids, as the temperature of the syrup was brought to the target temperature, under ambient (atmospheric) pressure.

Upon addition of the catalyst, the reaction was maintained at the target temperature under continuous mixing for about 2-4 hours, determined by following the reaction by HPLC. Next, the heat was turned off while maintaining constant stirring.

The reaction was then quenched by slowly adding approximately 60 mL of deionized (DI) water (room temperature) to dilute and cool the product mixture, to target a final concentration of 50-60 wt % dissolved solids. In some embodiments of this Example, the product mixture was brought to final concentration of 50-65 wt % dissolved solids. Generally, the water addition rate was performed to control the mixture viscosity as the oligosaccharide composition was cooled and diluted. Once quenched, the solid catalyst is filtered out using a fritted glass funnel.

### Example 5. De-Monomerization Procedure

Individual batches of the selected oligosaccharide composition, as produced in Examples 2-4, 12 and 13 were de-monomerized using one of two procedures as described below.

### Chromatography Method

Individual batches of the selected oligosaccharide composition were concentrated on a rotatory evaporator to approximately 50 Brix as measured by a Brix refractometer following treatment with ion-exchange resins (e.g., as described herein). The resulting syrup (up to 4 g dry basis) was loaded onto a Teledyne ISCO RediSep Rf Gold Amine column (55 grams stationary phase) using a luer-tip syringe. Other similar columns such as the Biotage SNAP KP-NH Cartridges may also be used. The sample was purified on a Biotage Isolera equipped with an ELSD detector using a 20/80 to 50/50 (v/v) deionized water/ACN mobile phase gradient over 55 column volumes. Other flash chromatography systems such as the Teledyne ISCO Rf may also be used. The flow rate was set in accordance with the manufacturer's specifications for the column and system. After the monomer fraction completely eluted at ˜16 column volumes, the mobile phase was set to 100% water until the remainder of the oligosaccharide composition eluted and was collected. The monomer-free fractions were concentrated by rotary evaporation to afford the de-monomerized product.

### Ethanol Precipitation

Individual batches of the selected oligosaccharide composition, as produced in Examples 2-4, 12 and 13 were concentrated on a rotatory evaporator to approximately 25 Brix as measured by a Brix refractometer following treatment with ion-exchange resins (e.g., as described herein). 100 mL of concentrated oligosaccharide composition, was poured into a vigorously stirred beaker containing 900 mL of pure, USP-grade ethanol at a rate no higher than 10 mL/minute. Once the addition was complete, the precipitated solids were subjected to stirring for an additional 15 minutes at or slightly below room temperature. The suspension was centrifuged at 4000 rpm for 4 hours at 5° C. The supernatant was decanted and the precipitated solids (pellets) were dissolved in water to a final concentration of 25 Brix and reconcentrated to >65 Brix. This syrup was then diluted back to 25 Brix and concentrated once more to ensure removal of residual ethanol. The resulting syrup was diluted back to 25 Brix, cooled to −78° C., and lyophilized to yield the demonomerized product.

### Example 6. Size Exclusion Chromatography

The weight-average molecular weight (MWw), number-average molecular weight (MWn), and polydispersity index (PDI) of batches of the selected oligosaccharide compositions, as described in Example 1, and produced according to the methods described in Examples 2-4, 12 and 13, were determined by SEC HPLC.

### Method

These methods involved the use of an Agilent 1100 with refractive index (RI) detector equipped with the following two columns in series: a Shodex OHpak SB-802 HQ, 8.0×300 mm, 8 μm, P/N F6429100 and a Shodex OHpak SB-803 HQ, 8.0×300 mm, 6 μm, P/N F6429102. Equivalent columns known in the art can also be used.

The mobile phase (0.1 M NaNO₃) was prepared by weighing 34 g of NaNO₃ (ACS grade reagent) and dissolving in 2000 mL of deionized (DI) water (from MiliQ water filter). The solution was filtered through a 0.2 μm filter.

Polymer standard solutions (10.0 mg/mL) were prepared by weighing 20 mg of a standard into a separate 20 mL scintillation vial and adding 2.0 mL of DI water to each vial.

Sample A was prepared in duplicate. Approximately 300 mg of oligosaccharide composition sample was weighed into a 20 mL scintillation vial and 10 mL of DI water was added. The solution was mixed and filtered through an Acrodisc 25 mm syringe filter with a 0.2 μm polyethersulfone membrane. Sample B was prepared in duplicate. Approximately 210 mg of oligosaccharide sample was weighed into a 20 mL scintillation vail and 10 mL of DI-water was added. The solution was mixed and filtered a Acrodisc 25 mm syringe filter with a 0.2 μm polyethersulfone membrane.

The flow rate was set to 0.7-0.9 mL/min at least 2 hours before running samples with the column temperature and RI detector each set to 40° C. with the RI detector purge turned on.

A blank sample consisting of DI water was run. Samples of each standard were run. Sample A was run. Sample B was run.

In some experiments, before running samples the injection volume for all samples was 10 μL and run time was 28 minutes, the detector purge was turned off and the pump was run at 0.7-0.9 mL/min until an acceptable baseline was obtained. The peaks between 15 and 22 minutes were integrated.

In other experiments, before running samples the injection volume for all samples was 10 μL and run time was 40 minutes, the detector purge was turned off and the pump was run at 0.7-0.9 mL/min until an acceptable baseline was obtained.

The calibration curve fit type in Empower 3 software was set to 3^{rd} order. The molecular weight distributions and polydispersity were calculated using Empower 3 software for the broad peak. The Mw, Mn and polydispersity of the product peak (DP2+) were reported.

### Results

Six batches of the selected oligosaccharide composition produced at the 10 kg scale using the process described in Example 3 were analyzed using the SEC methods described above. Small-scale batches of the selected oligosaccharide composition produced by the process described in Example 2 were de-monomerized.

The assayed batches of oligosaccharide composition produced according to Example 3 comprised oligosaccharides with an average MWw of 2443 g/mol (ranging from 2214-2715 g/mol), an average MWn of 1155 g/mol (ranging from 1095-1201 g/mol), and an average PDI of 2.1 (ranging from 2.0-2.3). Assayed batches further comprised an average DP2+ of 91.1% (ranging from 90.0-91.9) and an average degree of polymerization (DP) of 15.1 (ranging from 13.6-16.7).

Four batches of the selected oligosaccharide composition produced at the 500 kg (2000 L) scale using the process described in Example 13 were analyzed using the SEC methods described above. The assayed batches of oligosaccharide composition produced according to Example 13 comprised oligosaccharides with an average MWw of 2056 g/mol (ranging from 1968-2109 g/mol) and an average MWn of 1107 g/mol (ranging from 1071-1138 g/mol). Assayed batches further comprised an average DP2+ of 89.1% (ranging from 88.1-90.2%) and an average degree of polymerization (DP) of 12.7 (ranging from 12.1-13.0).

### Example 7. SEC HPLC Methodology for Determination of Impurities

The presence of residual organic acid impurities and related substances of batches and samples of the selected oligosaccharide composition, as produced by the process in Example 3, were determined by SEC HPLC.

### Methods

These methods involved the use of an Agilent 1100 with refractive index (RI) detector equipped with a guard column (Bio-Rad MicroGuard Cation H4-Cartridge, PIN 125-0129, or equivalent) and a Bio-Rad Aminex HPX-87H, 300×7.8 mm, 9 μm, PIN 125-0140 column, or equivalent.

The mobile phase (25 mM H₂SO₄ in water) was prepared by filling a bottle with 2000 mL DI-water and slowly adding 2.7 mL of H₂SO4. The solution was filtered through a 0.2 μm filter.

A standard solution was prepared by measuring 50±2 mg of reference standard into a 100-mL volumetric flask, adding mobile phase to 100-mL mark and mixing well.

A sample of a selected oligosaccharide composition (Sample A) was prepared in duplicate. Approximately 1000 mg of oligosaccharide sample was weighed into a 10 mL volumetric flask and mobile phase was added up to the mark. The solution was mixed and filtered through a PES syringe filter with a 0.2 μm polyethersulfone membrane.

A sample of a selected oligosaccharide composition (Sample B) was prepared in duplicate. Approximately 700 mg of oligosaccharide sample was weighed into a 10 mL volumetric flask and mobile phase was added up to the mark. The solution was mixed and filtered through a PES syringe filter with a 0.2 μm polyethersulfone membrane.

The flow rate was set to 0.65 mL/min at least 2 hours before running samples with the column temperature set to 50° C. and the RI detector temperature set to 50° C. with the RI detector purge turned on.

Before running samples the injection volume for all samples was 50 μL and run time was 40 minutes, the detector purge was turned off and the pump was run at 0.65 mL/min until an acceptable baseline was obtained.

A blank sample consisting of DI water was run. The standard, sample A, and sample B were each independently run.

The peaks at 7.5 min (Glucuronic acid), 9.4 min (Maleic Acid), 11.3 min (Levoglucosan), 11.9 min (Lactic Acid), 13.1 min (Formic Acid), 14.2 min (Acetic Acid), 15.5 min (Levulinic Acid), 31.8 min (hydroxymethylfurfural, HMF), and 8.3 min (Glucose) were integrated. The calibration curve fit type in Empower 3 software was set to 3^{rd} order.

### Results

Six batches of the selected oligosaccharide, as produced by the process in Example 3, were tested using the method above. Samples of the selected oligosaccharide composition comprised 0.19% w/w citric acid (ranging from 0.18-0.19% w/w) and undetectable levels of lactic acid, formic acid, levulinic acid and HMF.

### Example 8. HSQC NMR Analysis Procedure Using a Bruker NMR Machine

A determination of HSQC NMR spectra of samples of the selected oligosaccharide compositions described in Example 1, and produced as described in Examples 2 and 3, was performed using a Bruker NMR machine, according to the protocol described below.

### Method

### Sample Preparation:

30 mg of a previously lyophilized solid sample of the oligosaccharide composition was dissolved in 300 μL of D₂₀ with 0.1% acetone as internal standard. The solution was then placed into a 3 mm NMR tube.

### NMR Experiment:

Each sample was analyzed in a Bruker NMR operating at 499.83 MHz (125.69 MHz 13C) equipped with a XDB broadband probe with Z-axis gradient, tuned to 13C, and operating at 25° C. Each sample was subjected to a multiplicity-edited gradient-enhanced 1H-13C heteronuclear single quantum coherence (HSQC) experiment using the echo-antiecho scheme for coherence selection. The following pulse sequence diagram and acquisition and processing parameters were used to obtain the NMR spectrum for each sample:

### Spectral Analysis

The resulting spectra were analyzed using the MNova software package from Mestrelab Research (Santiago de Compostela, Spain). The spectra were referenced to the internal acetone signal (1H-2.22 ppm; ¹³C-30.8 ppm) and phased using the Regions2D method in both the F2 and F1 dimension. Apodization using 90 degree shifted sine was applied in both the F2 and F1 dimension. For each spectrum, individual signals (C—H correlations) were quantified by integration of their respective peaks using “predefined integral regions” with elliptical shapes. FIG. 6B shows the integral regions that define the coordinates of HSQC NMR peaks/signals 1-11 of the selected oligosaccharide composition. FIG. 6C shows an example of an elliptical shape defined by major axis coordinates (F2 dimension; ¹H) and minor axis coordinates (F1 dimension; ¹³C). The resulting table of integral regions and values from the spectra were normalized to a sum of 100 in order for the value to represent a percentage of the total. The peak integral regions were selected to avoid peaks associated with monomers and to focus on the distinguishing features of the spectrum.

### Results

Six batches of the selected oligosaccharide composition produced according to the process described in Example 3 (having an average DP of 15.1 (+1.3)) and two batches of the selected oligosaccharide composition produced according to the process described in Example 2 were analyzed using the NMR methods described above.

Samples of the batches of the selected oligosaccharide composition were analyzed by HSQC NMR after being demonomerized according to the ethanol precipitation procedure as described in Example 5. Samples of the batches of the selected oligosaccharide composition were also analyzed by HSQC NMR without being demonomerized. Notably, analysis of HSQC NMR spectra of samples before and after demonomerization provided substantially similar peaks (e.g., similar relative AUC values at signals 1-11). Table 9 provides the predefined integral regions, or coordinates that bound (i.e., define) the elliptical shapes.

TABLE 9
Coordinates of HSQC NMR integral regions for
the selected oligosaccharide composition

| | ¹H Position (ppm) | | | ¹³C Position (ppm) | | |
|---|---|---|---|---|---|---|
| | Center | ¹H Integral Region | | Center | ¹³C Integral Region | |
| Signal | Position | from | to | Position | From | to |
| 1 | 5.04 | 5.08 | 5.01 | 108.38 | 108.06 | 108.70 |
| 2 | 5.02 | 5.04 | 5.00 | 101.68 | 101.47 | 101.90 |
| 3 | 4.98 | 5.02 | 4.94 | 98.99 | 98.40 | 99.58 |
| 4 | 4.81 | 4.82 | 4.79 | 101.2 | 100.99 | 101.42 |
| 5 | 4.6 | 4.62 | 4.58 | 104.96 | 104.74 | 105.17 |
| 6 | 4.45 | 4.50 | 4.40 | 103.84 | 103.46 | 104.21 |
| 7 | 4.19 | 4.21 | 4.16 | 82.1 | 81.72 | 82.48 |
| 8 | 4.14 | 4.19 | 4.10 | 76.91 | 76.43 | 77.39 |
| 9 | 4.11 | 4.17 | 4.05 | 81.72 | 81.35 | 82.10 |
| 10 | 4.06 | 4.11 | 4.02 | 77.25 | 76.61 | 77.90 |
| 11 | 3.98 | 4.03 | 3.93 | 83.52 | 83.09 | 83.95 |

The relative size of each of the peaks (area under the curve (AUC)) collected for the NMR spectra of the eight total batches of the selected oligosaccharide composition produced according to the processes as described in Examples 2 and 3 (and not demonomerized) was further determined, as shown below in Table 10:

TABLE 10
HSQC NMR data (batches produced using processes as
described in Examples 2 and 3; not de-monomerized)

| | | | Mean +3 std | | Mean −3 std |
|---|---|---|---|---|---|
| | | | dev. AUC | Mean AUC | dev. AUC |
| | Center Position (ppm) | | (% of total areas | (% of total areas | (% of total areas |
| Signal | ¹H | ¹³C | of signals 1-11) | of signals 1-11) | of signals 1-11) |
| 1 | 5.04 | 108.38 | 5.92 | 5.25 | 4.57 |
| 2 | 5.02 | 101.68 | 1.58 | 1.20 | 0.81 |
| 3 | 4.98 | 98.99 | 20.43 | 15.79 | 11.16 |
| 4 | 4.81 | 101.2 | 1.78 | 1.40 | 1.02 |
| 5 | 4.6 | 104.96 | 4.09 | 2.26 | 0.43 |
| 6 | 4.45 | 103.84 | 14.37 | 11.16 | 7.94 |
| 7 | 4.19 | 82.1 | 5.56 | 4.68 | 3.81 |
| 8 | 4.14 | 76.91 | 12.67 | 10.49 | 8.30 |
| 9 | 4.11 | 81.72 | 18.42 | 15.49 | 12.55 |
| 10 | 4.06 | 77.25 | 25.79 | 21.64 | 17.49 |
| 11 | 3.98 | 83.52 | 11.27 | 10.32 | 9.38 |

The relative size of each of the peaks (area under the curve (AUC)) collected for the NMR spectra of the eight total batches of the selected oligosaccharide composition produced according to the processes as described in Examples 2 and 3 and demonomerized according to the ethanol precipitation procedure as described in Example 5 was further determined, as shown below in Table 11:

TABLE 11
HSQC NMR data (batches produced using processes
as described in Examples 2 and 3; de-monomerized)

| | | | Mean +5 std | Mean +3 std | | Mean −3 std | Mean −3 std |
|---|---|---|---|---|---|---|---|
| | | | dev. AUC | dev. AUC | Mean AUC | dev. AUC | dev. AUC |
| | Center | | (% of total | (% of total | (% of total | (% of total | (% of total |
| | Position (ppm) | | areas of | areas of | areas of | areas of | areas of |
| Signal | ¹H | ¹³C | signals 1-11) | signals 1-11) | signals 1-11) | signals 1-11) | signals 1-11) |
| 1 | 5.04 | 108.38 | 6.75 | 6.18 | 5.32 | 4.46 | 3.89 |
| 2 | 5.02 | 101.68 | 2.01 | 1.70 | 1.23 | 0.77 | 0.46 |
| 3 | 4.98 | 98.99 | 26.02 | 22.14 | 16.33 | 10.52 | 6.65 |
| 4 | 4.81 | 101.2 | 2.14 | 1.85 | 1.41 | 0.98 | 0.69 |
| 5 | 4.6 | 104.96 | 4.06 | 3.24 | 2.00 | 0.77 | 0.00 |
| 6 | 4.45 | 103.84 | 16.82 | 14.63 | 11.35 | 8.06 | 5.87 |
| 7 | 4.19 | 82.1 | 5.35 | 5.01 | 4.50 | 3.98 | 3.64 |
| 8 | 4.14 | 76.91 | 14.56 | 12.89 | 10.39 | 7.88 | 6.21 |
| 9 | 4.11 | 81.72 | 20.64 | 18.67 | 15.73 | 12.78 | 10.82 |
| 10 | 4.06 | 77.25 | 29.79 | 26.28 | 21.02 | 15.75 | 12.24 |
| 11 | 3.98 | 83.52 | 12.41 | 11.59 | 10.37 | 9.14 | 8.32 |

The relative size of each of the peaks (area under the curve (AUC)) collected for the NMR spectra of the six total batches of the selected oligosaccharide composition produced according to the processes as described in Example 3 (and not demonomerized) was further determined, as shown below in Table 12:

TABLE 12
HSQC NMR data (batches produced using processes as described in Example 3)

| | | | Mean +3 std | | Mean −3 std |
|---|---|---|---|---|---|
| | | | dev. AUC | Mean AUC | dev. AUC |
| | Center Position (ppm) | | (% of total areas | (% of total areas | (% of total areas |
| Signal | ¹H | ¹³C | of signals 1-11) | of signals 1-11) | of signals 1-11) |
| 1 | 5.04 | 108.38 | 5.93 | 5.18 | 4.43 |
| 2 | 5.02 | 101.68 | 1.58 | 1.21 | 0.84 |
| 3 | 4.98 | 98.99 | 18.46 | 16.55 | 14.65 |
| 4 | 4.81 | 101.2 | 1.66 | 1.45 | 1.24 |
| 5 | 4.6 | 104.96 | 3.45 | 2.61 | 1.77 |
| 6 | 4.45 | 103.84 | 13.51 | 11.68 | 9.85 |
| 7 | 4.19 | 82.1 | 5.63 | 4.74 | 3.85 |
| 8 | 4.14 | 76.91 | 11.33 | 10.23 | 9.14 |
| 9 | 4.11 | 81.72 | 15.97 | 15.02 | 14.07 |
| 10 | 4.06 | 77.25 | 22.44 | 21.06 | 19.67 |
| 11 | 3.98 | 83.52 | 10.60 | 10.19 | 9.78 |

Samples of commercially available oligosaccharides (galactooligosaccharide, lactose, melibiose, the human milk oligosaccharide 2-a-L-fucopyranosyl-D-lactose, and the human milk oligosaccharide Lacto-N-neotetraose) were analyzed by HSQC NMR as described above. NMR spectra of each commercially available oligosaccharide was overlaid with the assigned peak integral regions of the selected oligosaccharide (i.e., peak signals 1-11 in Table 9) and the relative AUC of each of those integral regions were determined, as shown below. These experiments demonstrate that the HSQC NMR spectra of the selected oligosaccharide composition is significantly different than those of tested commercially available oligosaccharides (Table 13).

TABLE 13
HSQC NMR data of commercially available oligosaccharides using
integral regions for the selected oligosaccharide composition

| | | | galactooligo- | Lactose | | 2-a-L- | Lacto-N- |
|---|---|---|---|---|---|---|---|
| | | | saccharide | (% of | Melibiose | fucopyranosyl- | neotetraose |
| | Center | | (% of total | total | (% of | D-lactose (% | (% of total |
| | Position (ppm) | | areas of | areas of | total areas | of total areas | areas of |
| Signal | ¹H | ¹³C | signals 1-11) | signals 1-11) | of signals 1-11) | of signals 1-11) | signals 1-11) |
| 1 | 5.04 | 108.38 | NS | NS | NS | NS | NS |
| 2 | 5.02 | 101.68 | NS | NS | NS | NS | NS |
| 3 | 4.98 | 98.99 | NS | NS | 98.96 | NS | NS |
| 4 | 4.81 | 101.2 | NS | NS | NS | NS | NS |
| 5 | 4.6 | 104.96 | 31.36 | NS | NS | NS | NS |
| 6 | 4.45 | 103.84 | 65.39 | 98.34 | NS | NS | 97.77 |
| 7 | 4.19 | 82.1 | NS | NS | NS | NS | NS |
| 8 | 4.14 | 76.91 | NS | NS | NS | NS | NS |
| 9 | 4.11 | 81.72 | NS | NS | NS | NS | NS |
| 10 | 4.06 | 77.25 | NS | NS | NS | NS | NS |
| 11 | 3.98 | 83.52 | NS | NS | NS | NS | NS |

*NS = no peak signal detected

### Example 9. Determination of Glycosidic Bond Distribution Using Permethylation Analysis

A determination of glycosidic bond distribution of samples of the selected oligosaccharide composition, as produced by the process in Example 2, was performed using permethylation analysis, according to the protocol described below. Samples were demonomerized prior to permethylation analysis.

Reagents used were methanol, acetic acid, sodium borodeuteride, sodium carbonate, dichioromethane, isopropanol, trifluoroacetic acid (TFA), and acetic anhydride. Equipment included a heating block, drying apparatus, gas chromatograph equipped for capillary columns and with a RID/MSD detector, and a 30 meter RTX®-2330 (RESTEK). All derivation procedures were done in a hood.

### Preparation of Alditol Acetates

a. Standard Preparation

1 mg/mL solutions of the following standard analytes were prepared: arabinose, rhamnose, fucose, xylose, mannose, galactose, glucose, and inositol. The standard was prepared by mixing 50 μL of each of arabinose, xylose, fucose, glucose, mannose, and galactose with 20 μL of inositol in a vial. The standard was subsequently lyophilized.

### B. Sample Preparation

Each sample was prepared by mixing 100-500 μg of the selected oligosaccharide composition (as weighed on an analytical balance) with 20 μg (20 μL) of inositol in a vial.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with N₂/air. 200 μL of IPA (isopropanol) was added and dried down again with N₂/air. This hydrolysis step (addition of TEA for two hours at 121° C.; washing with isopropanol) was repeated twice.

The standard was similarly subjected to hydrolysis using TFA, as described for the sample.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH: HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

### Glycosyl-Linkage Analysis of Poly- and Oligosaccharides by Hakomori Methylation

a. Preparation of NaOH Base

In a glass screw top tube, 100 μL of a 50/50 NaOH solution and 200 μL of dry MeOH were combined. Plastic pipets were used for the NaOH and glass pipets were used for the MeOH. The solution was vortexed briefly, approximately 4 mL dry DMSO was added, and the solution was vortexed again. The tube was centrifuged to concentrate the solution and the DMSO and salts were pipetted off from the pellet. The previous two steps were repeated about four times in order to remove all the water from the pellet. All white reside was removed from the sides of the tube. Once all the residue was removed and the pellet was clear, about 1 mL dry DMSO was added and the solution was vortexed. The base was then ready to use. The base was prepared fresh each time it was needed.

### B. Permethylation

Each sample was prepared by mixing 600-1000 μg of the selected oligosaccharide composition (as weighed on an analytical balance) with 200 μL DMSO. The sample was stirred overnight until the oligosaccharide composition dissolved.

An equal amount of NaOH base (400 μL) was added to the sample, after which the sample was placed back on the stirrer and mixed well for 10 minutes. 100 μL of iodomethane (CH₃D) was added to the sample. The sample was mixed on the stirrer for 20 minutes, and then the previous steps (addition of NaOH base and iodomethane) were repeated.

Approximately 2 mL ultrapure water was added to the sample and the sample was mixed well, such that it turned cloudy. The tip of a pipette was placed into the sample solution at the bottom of the tube and CH₃I was bubbled off with a very low flow of air. The sample became clear as the CH₃I was bubbled off. The pipette was moved around the solution to make certain that all the CH₃I was gone. Approximately 2 mL methylene chloride was then added and the solution was mixed well by vortex for 30 seconds. The sample was then centrifuged and the top aqueous layer was removed. Approximately 2 mL of water were added and the sample was mixed, then briefly centrifuged, then the top aqueous layer was removed. The additions of methylene chloride and water were repeated. The organic bottom layer was removed and transferred into another tube and dried down using N₂. The analysis was continued with Alditol Acetates.

### C. Hydrolysis

200 μL of 2 M tifluoroacetic acid (TFA) was added to the sample(s). The vial containing the sample was capped tightly and incubated on a heating block for 2 hours at 121° C. After 2 hours, the sample was removed from the heating block and allowed to cool to room temperature. The sample was then dried down with N₂/air. 200 μL of IPA (isopropanol) was added and dried down again with N₂/air. This hydrolysis step (addition of TFA for two hours at 121° C.; washing with isopropanol) was repeated twice.

### D. Reduction and Acetylation

10 mg/mL solution of sodium borodeuteride was prepared in 1 M ammonium hydroxide. 200 μL of this solution was added to the sample. The sample was then incubated at room temperature for at least one hour or overnight. After incubation with sodium borodeuteride solution, 5 drops of glacial acetic acid were added to the sample, followed by 5 drops of methanol. The sample was then dried down. 500 μL of 9:1 MeOH: HOAc was added to the sample and subsequently dried down (twice repeated). 500 μL MeOH was then added to the sample and subsequently dried down (once repeated). This produced a crusty white residue on the side of the sample vial.

250 μL acetic anhydride was then added to the sample vial and the sample was vortexed to dissolve. 230 μL concentrated TFA was added to the sample and the sample was incubated at 50° C. for 20 minutes. The sample was removed from the heat and allowed to cool to room temperature. Approximately 1 mL isopropanol was added and the sample was dried down. Then, approximately 200 μL isopropanol was added and the sample was dried down again. Approximately 1 mL of 0.2M sodium carbonate was then added to the sample and it was mixed gently. Approximately 2 mL dichloromethane was finally added to the sample, after which it was vortexed and centrifuged briefly. The aqueous top layer was discarded. 1 mL water was added and the sample was vortexed and centrifuged briefly. This step was repeated before the organic layer (bottom) was removed and transferred to another vial. The sample was concentrated using N₂/air to a final volume of about 100 μL. 1 μL of final sample was then injected on GC-MS.

The GC temperature program SP2330 was utilized for GC-MS analysis. The initial temperature was 80° C. and the initial time was 2.0 minutes. The first ramp was at a rate of 30° C./min with a final temperature of 170° C. and a final time of 0.0 minutes. The second ramp was at a rate of 4° C./min with a final temperature of 240° C. and a final time of 20.0 minutes.

### Results

Permethylation data was collected using the methods described above for six batches of de-monomerized oligosaccharide composition produced by the process described in Example 3. Each batch was analyzed in duplicate. Data relating to the radicals present in these six batches of de-monomerized oligosaccharide composition are provided below in Table 14:

TABLE 14
Permethylation data (de-monomerized oligosaccharide composition
produced by the process described in Example 3)

| | Mean | | Mean |
|---|---|---|---|
| | mol | | mol |
| | % −3 | Mean | % +3 |
| | standard | mol | standard |
| Radicals | dev. | % | dev. |
| t-galactofuranose monoradicals | 6.29% | 9.56% | 12.84% |
| t-galactopyranose monoradicals | 20.45% | 24.51% | 28.58% |
| 2-galactofuranose and/or 2- | 2.73% | 3.09% | 3.46% |
| glucofuranose monoradicals | | | |
| 3-galactopyranose monoradicals | 5.31% | 6.23% | 7.15% |
| 3-galactofuranose monoradicals | 3.36% | 3.82% | 4.28% |
| 2-galactopyranose monoradicals | 4.12% | 4.29% | 4.45% |
| 4-galactopyranose and/or 5- | 4.65% | 5.26% | 5.87% |
| galactofuranose monoradicals | | | |
| 2,3-galactofuranose diradicals | 0.43% | 0.62% | 0.82% |
| 6-galactofuranose monoradicals | 2.92% | 6.25% | 9.58% |
| 6-galactopyranose monoradicals | 13.81% | 16.42% | 19.02% |
| 3,4-galactopyranose and/or 3,5- | 1.41% | 1.70% | 1.99% |
| galactofuranose and/or 2,3- | | | |
| galactopyranose diradicals | | | |
| 2,4-glucopyranose and/or 2,5- | 0.88% | 1.05% | 1.21% |
| glucofuranose and/or 2,4-galactopyranose | | | |
| and/or 2,5-galactofuranose diradicals | | | |
| 2,3,4-galactopyranose and/or 2,3,5- | 0.14% | 0.21% | 0.28% |
| galactofuranose triradicals | | | |
| 3,6-galactofuranose diradicals | 1.69% | 1.98% | 2.27% |
| 4,6-galactopyranose and/or 5,6- | 3.93% | 4.60% | 5.26% |
| galactofuranose diradicals | | | |
| 3,6-galactopyranose and/or 2,6- | 4.14% | 5.03% | 5.93% |
| galactofuranose diradicals | | | |
| 2,6-galactopyranose diradicals | 1.98% | 2.49% | 2.99% |
| 3,4,6-galactopyranose and/or 3,5,6- | 0.91% | 1.30% | 1.68% |
| galactofuranose and/or 2,3,6- | | | |
| galactofuranose triradicals | | | |
| 2,3,6-galactopyranose and/or 2,4,6- | 0.01% | 1.54% | 3.10% |
| galactopyranose and/or 2,5,6- | | | |
| galactofuranose triradicals | | | |
| 2,3,4,6-galactopyranose and/or 2,3,5,6- | 0.01% | 0.04% | 0.28% |
| galactofuranose quadradicals | | | |

### Example 10. The Selected Oligosaccharide Composition Increases SCFA Production in Ex Vivo Fecal Samples

The ability of the selected oligosaccharide composition comprised of a plurality of oligosaccharides selected from Formula (I) as produced by a process as described in Example 3 to increase the production of short-chain fatty acids (SCFAs) in fecal suspensions of healthy subjects was assessed.

Eight fecal samples from healthy subjects were collected, frozen and stored at −80° C. until use. Frozen fecal samples were transferred to an anaerobic chamber, allowed to thaw and homogenized to a final concentration of 20% solids in phosphate buffer saline (PBS) supplemented with 15% glycerol. The 20% fecal slurries were then filtered to remove large debris, aliquoted, removed from the anaerobic chamber and immediately frozen on dry ice before storage at −80° C. On the day of the experiment, one 1 mL aliquot of each 20% fecal slurry was thawed and diluted into bacterial growth media (Clostridium minimal medium supplemented with 0.1% w/v trypticase peptone and 0.75 mM urea) to a final concentration of 1% in an anaerobic chamber. The 1% fecal slurry solutions were then dispensed into the wells of 96 deep well plates containing sterile water (negative control) or preparations of the oligosaccharide preparations (final concentration of 0.5%). Three replicates of each sample were prepared. Fecal microbial cultures were incubated anaerobically at 37° C. for 45 hours.

After the 45-hour incubation period, the 96-well deep well plates containing the fecal microbial cultures are removed from the anaerobic chamber and kept on ice. The plates were sedimented by centrifugation (3,000×g) for 10 minutes at 4° C. Fecal microbiota culture supernatants and pellets were collected and stored at −80° C. Supernatant samples were thawed and analyzed by gas chromatography with a flame ionization detector (GC-FID) to quantify the concentration of short-chain fatty acids (acetate, propionate, and butyrate) produced in the fecal samples.

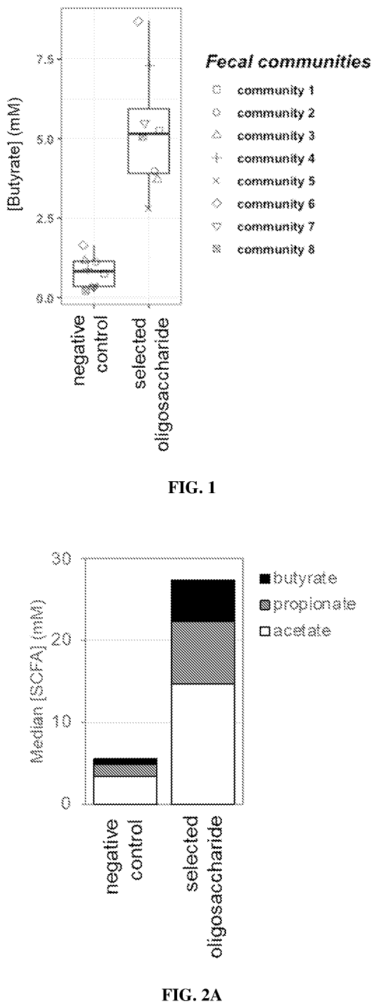

Incubation of the fecal communities with the selected oligosaccharide composition resulted in the median production of approximately 5.1 mM total SCFA (an increase of 4.6 mM compared to the negative control) across the tested fecal communities (FIG. 1). Notably, the selected oligosaccharide composition increased butyrate production in each of the eight fecal communities.

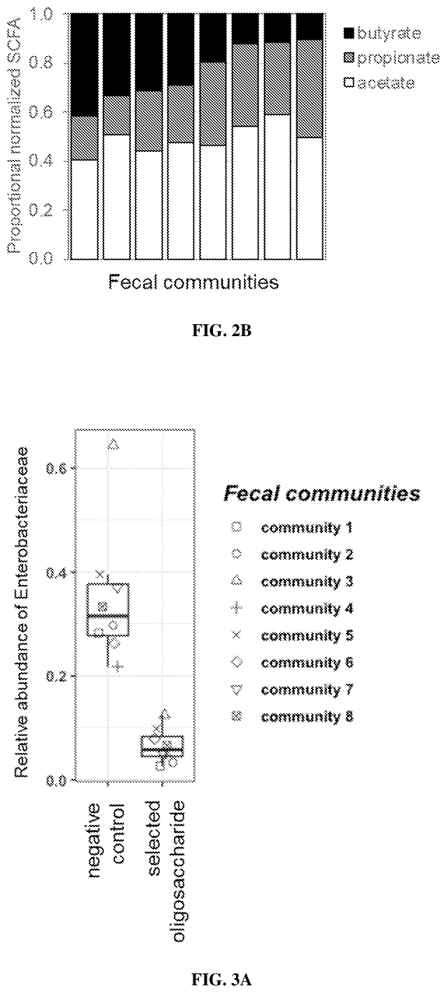

In addition to butyrate, the selected oligosaccharide composition also increased the production of acetate and propionate from these fecal communities. Incubation of the fecal communities with the selected oligosaccharide composition resulted in the production a median concentration of 27.4 mM total SCFA (i.e., the sum of acetate, propionate, and butyrate) (FIG. 2A). Conversely, the negative control led to the production of only 5.6 mM total SCFA. This increase in total SCFA production caused by the selected oligosaccharide composition showed variation in the relative proportions of butyrate and propionate. In four of the eight fecal communities incubated with the selected oligosaccharide composition, between 20 and 40% of the total SCFA produced was butyrate, while propionate comprised a smaller proportion of the total (FIG. 2B). In the other four fecal communities, propionate made up a larger proportion of the total SCFA produced than did butyrate.

To determine how the selected oligosaccharide composition changed the taxonomic composition of the fecal communities, fecal microbial culture pellets were subjected to 16S rRNA amplicon sequencing. Culture pellets were thawed and subjected to genomic DNA (gDNA) extraction using for all using the MagAttract PowerMicrobiome DNA/RNA Kit (Qiagen) according to the manufacturer's instructions. gDNA was quantitated using the Quant-iT Picogreen dsDNA assay kit (Thermo Fisher Scientific) and normalized to 1 ng/u L. Polymerase chain reaction (PCR) and sequencing were performed using a modified protocol described previously (Caporaso et al. 2011). Briefly, PCR products were amplified using barcoded primers targeting the V4 region of the 16S rRNA gene. PCR products were subsequently purified using the AMPure XP PCR purification system (Beckman Coulter). PCR products were purified, run on a 1% agarose gel, stained with ethidium bromide, and imaged to ensure the presence of the correct amplicon. Purified PCR concentrations were quantitated (as described above for gDNA), normalized to a concentration of 4 nM, and pooled to create a final library consisting of 5 μL of each amplicon. 16S rRNA sequencing was conducted on an Illumina Miseq using a MiSeq reagent kit v2 (500 cycles) as outlined in Caporaso et al. 2011.

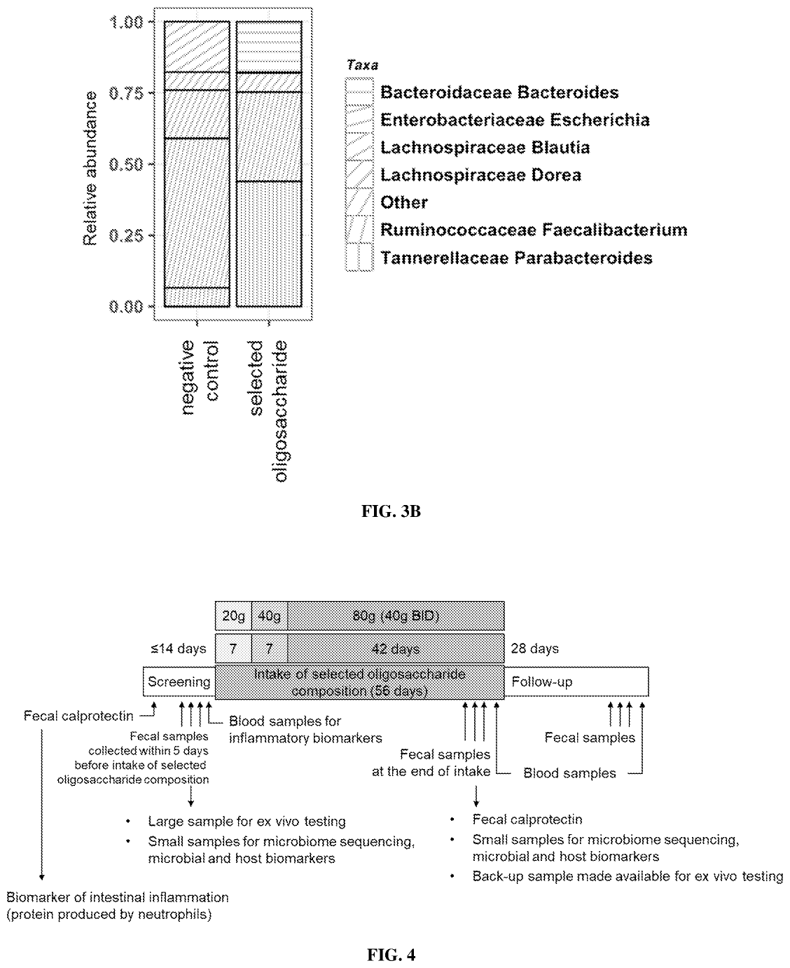

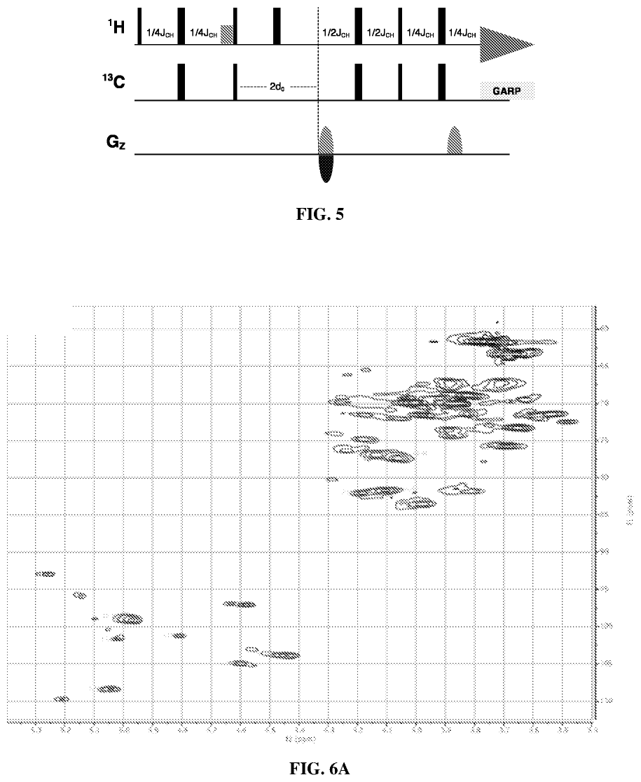

The selected oligosaccharide composition significantly decreased the relative abundance of the pathobiont family Enterobacteriaceae compared to the negative control in each of the eight communities tested (FIG. 3A). The selected oligosaccharide composition also significantly increased the relative abundance of the commensal genera Parabacteroides and Bacteroides (FIG. 3B).

These data collectively demonstrate that the selected oligosaccharide composition robustly and consistently increases total SCFA; and increases the amounts of commensal bacteria (i.e., Parabacteroides and Bacteroides) while decreasing the amounts of pathobionts (i.e., Enterobacteriaceae).

### Example 11. Human Trial to Assess Safety and Tolerability of the Selected Oligosaccharide Composition in Patients with Ulcerative Colitis Disease

The safety and tolerability of the selected oligosaccharide composition as produced by a process as described in Examples 3-4, 12 and 13 was assessed in patients with ulcerative colitis (UC). Additional observations included changes in the patient's microbiome as well as selected biomarkers and health assessments. Eligible patients (ages 18-75) had mild-to-moderate UC symptoms while on treatment with oral mesalamine and/or purine analogs.

10 patients with mild-to-moderate UC symptoms were entered into the study. The study consisted of a Screening Period (14±2 Days), a Treatment Period (56±3 Days), and a Follow-up Period (28±3 Days), as detailed in FIG. 4.

Patients who met inclusion and exclusion criteria were eligible to enter a 14 day +2 screening period, where blood and stool samples were collected, and a 3-day diet diary was completed.

Inclusion criteria included the following:
Be male or female, ≥18 and ≤75 years of age
Have a body mass index ≥18.5 and <45 kg/m²
Confirmed diagnosis of UC (>1 year) by endoscopy
Mild-to-moderate UC symptoms with ≥3 and <8 bowel movements per day within 1 week of screening
At least 4 weeks of UC symptomatology prior to the screening visit based on principal investigator (PI) judgement
Patients remained on a stable medication regimen for 2 weeks prior to randomization if they were taking medications for ulcerative colitis

Exclusion criteria included:
Possible or confirmed diagnosis of Crohn's disease or indeterminate disease
Prior medical history of isolated distal proctitis limited to the rectum due to IBD or other etiologies, e.g., sexually transmitted infections, anal trauma, salmonella and/or shigella infections.
Antibiotic treatment within the past 28 days prior to screening
Any non-UC related immunosuppressive or autoimmune condition, or treatment for any non-UC related immunosuppressive or autoimmune condition is excluded; systemic corticosteroids >prednisone 10 mg QD was excluded
Any immunosuppressive condition or treatment with immunosuppressive medications other than purine analog and no systemic corticosteroids, as defined
Patients with an initial fecal calprotectin <250 μg/g

Patients were scheduled for the Baseline (Day 1) in-person visit to complete safety assessments. The 3-day diet diary was returned, and the patients bad blood drawn and stool samples collected in order to assess biochemical markers and microbiome composition. The Simple Clinical Colitis Activity Index (SCCAI) composite score and the mean Fatigue Severity Scale (FSS) were administered. After completion of these assessments, patients were instructed to consume the first dose of the selected oligosaccharide composition, under supervision. Patients were then dismissed and continued to take the selected oligosaccharide composition orally twice daily at home, with dosage up titration as delineated below.

Patients completed the 3-day diet diary for the week preceding each study visit and attended the screening (Day −14±2 days), baseline (Day 1), end of treatment (Day 56) and follow-up (Day 84±2 days). Logs documenting any adverse effects were completed daily by the patient at home and reviewed by site staff at each in-person, telephone, and virtual visit.

On Day 30 (±2 days), patients were interviewed to confirm study compliance and assess any changes in health status or treatment-emergent adverse effects (TEAEs) and the SCCAI was administered.

On the last day of treatment (Day 56; end of intake period), patients returned the 3-day diet diary and TEAE logs were reviewed; stool samples and blood were collected; the SCCAI and FSS administered, then patients entered a 28 day follow up period. During the follow-up period, patients completed the 3-day diet diary and TEAE logs. Stool samples were collected at the end of the follow-up period to assess biochemical markers and changes to microbiome composition.

After day 84, subjects had completed the main study. Patients who completed through Day 84 were contacted 8 weeks later to complete a UC Extension Questionnaire, the SCCAI and to provide stool and blood samples (blood samples were collected if feasible) to assess the effect of withdrawal of the selected oligosaccharide composition 3 months after study product intake on biomarkers of inflammation and microbiome structure and function.

On Days 14 and 44 (both ±3 days), patients received telephone calls to review study compliance and assess any changes in health status or TEAEs.

Patients consumed the selected oligosaccharide composition orally after reconstitution in at least 120 mL of water for each dose administered, twice daily, according to the following dosing schedule:

| | |
|---|---|
| Period 1 (Days 1-7) | 20 g consumed (i.e, 10 g twice daily) |
| Period 2 (Days 8-14) | 40 g consumed (i.e, 20 g twice daily) |
| Period 3 (Days 15-55) | 80 g consumed (i.e, 40 g twice daily) |
| Period 3 (Day 56) | 40 g (morning dose only) |

Safety and tolerability were determined based on:
Number of patients experiencing any treatment-emergent adverse events (TEAEs) including causality, severity, and seriousness assessments
Tolerability: treatment-related adverse events (AEs) and discontinuations due to AEs
Change from baseline in:
   Vital signs
   Safety laboratory analysis
Physical Examinations

Exploratory endpoint parameters included:
Change from baseline in alpha diversity, taxonomy, and abundance of bacteria (including Enterobacteriaceae; relative to total, and per gram of stool) as measured by nucleic acid sequencing to the end of study treatment
Change from baseline to the end of intake period in biochemical and other biomarkers of inflammation in stool and blood
Measurement of the relative abundance of bacterial taxa (i.e., gut microbiome composition) after incubation of clinical run-in samples with the selected oligosaccharide composition in using the ex vivo culture system compared to those observed between run-in and end of intake in subjects.
Change from baseline to the end of intake period in Simple Clinical Colitis Activity Index (SCCAI) composite score
Change from baseline to the end of intake period in the mean Fatigue Severity Scale (FSS) score
Proportion of patients with fatigue (defined as mean FSS score ≥4) to the end of intake period in the mean FSS score
Proportion of patients with fatigue (defined as mean FSS score ≥4) to the end of follow-up period in the mean FSS score
Healthcare utilization such as hospitalizations, non-routine visits to healthcare professionals for UC

### Results

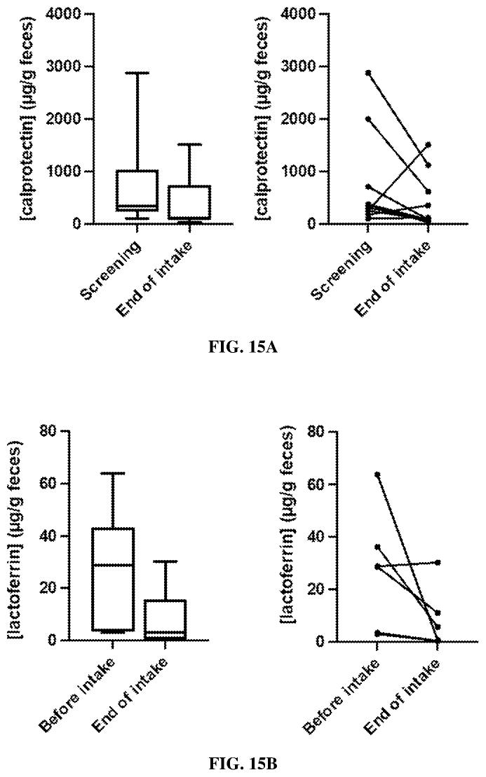

Calprotectin was measured in fecal samples collected from patients at screening and at the end of the intake period using the EliA Calprotectin 2 test (Phadia Laboratory Systems). The concentration of fecal calprotectin decreased from screening to the end of intake by a median value of 68.7% across ten participants examined in the study (FIG. 15A). The fecal calprotectin decreased by at least 50% in seven out of the ten participants. Table 18 provides the levels of fecal calprotectin (μg/g feces) for each participant at the initial screening (before administration of the selected oligosaccharide composition), at the end of intake (after administration of the selected oligosaccharide composition, and includes the percent change.

TABLE 18
Fecal calprotectin (μg/g feces) for each participant

| | Screening | End of intake | Percent change from |
|---|---|---|---|
| Participant | (visit 1) | (visit 6) | screening to end of intake |
| 1 | >2000 | 620 | −69.0 |
| 2 | >2000 | 1120 | −61.1 |
| | (2882, | | |
| | visit 2) | | |
| 3 | 257 | 81 | −68.5 |
| 4 | 712 | 87 | −87.8 |
| 5 | 310 | 90 | −71.0 |
| 6 | 367 | 36 | −90.2 |
| 7 | 258 | 1514 | 486.8 |
| 8 | 107 | 126 | 17.8 |
| 9 | 182 | 359 | 97.3 |

Calprotectin is a protein which is found in cells involved in the immune responses to pathogens, such as neutrophil, monocytes, and macrophages (Gaya et al, 2002, Q J Med; Roseth et al, 2004, Scand J Gastroenterol). It can account for as much as 60% of the cytoplasmic proteins in neutrophils. During intestinal inflammation, neutrophils migrate through the intestinal epithelium into the intestinal lumen, leading to increased quantities of calprotectin in the stool (Masoodi et al, Ger Med Sci. 2011 Feb. 16; 9: Doc03.). The level of fecal calprotectin correlates with the number of neutrophils in the intestinal lumen and is elevated in inflammatory bowel diseases (IBD), such as Crohn's disease and ulcerative colitis (Konikoff M. R., Inflamm Bowel Dis. 2006 June; 12 (6): 524-34).

Two other protein biomakers of intestinal inflammation were also measured in fecal samples collected from patients at screening (before intake) and at the end of the intake period. Fecal lactoferrin and lipocalin were measured using ELISA assays (BioVendor). Fecal lactoferrin concentration decreased from screening to the end of intake by a median value of 69.2% across six participants tested (FIG. 15B). The fecal lactoferrin concentrations decreased by at least 50% in five out the six participants. Fecal lactoferrin has been shown to be a specific and selective biomarker of IBD disease activity (Dai et al, Scand J Gastroenterol., Volume 42, 2007-Issue 12, Pages 1440-1444, 2007).

Fecal lipocalin concentrations showed a trend towards decreased levels, decreasing in three out of the six participants.

These decreases in fecal biomarkers of inflammation suggest a reduction in local intestinal inflammation resulting from the administration of the selected oligosaccharide composition to patients having ulcerative colitis, suggesting the selected oligosaccharide compositions described herein may be useful for treatment of patients with inflammatory diseases such as inflammatory bowel diseases, including UC. with.

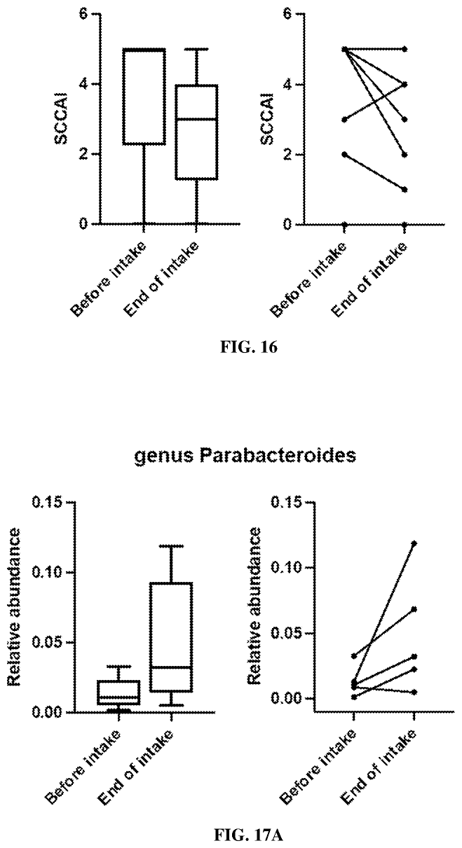

Disease activity was also assessed in study participants using the Simple Clinical Colitis Activity Index (SCCAI) before and at the end of the intake period. SCCAI decreased in five out of the eight participants evaluated using this measure (FIG. 16).

Additional protein biomarkers of inflammation were also measured in the plasma samples of five study participants. These biomarkers included high-sensitivity C-reactive protein (bsCRP), calprotectin, lipocalin, and LPS-binding protein (LBP). A biomarker of intestinal epithelial integrity, intestinal fatty acid-binding protein (I-FABP) was also measured in plasma samples. Small changes in these plasma biomarkers were observed after the intake period. However, the level of each biomarker before intake was generally low and within ranges expected for healthy subjects.

A panel of cytokines as biomarkers of systemic inflammation were measured in plasma samples. This panel included TNFα, IL-1β, IL-6, IL-12, IFNγ, IL-2, IL-4, IL-13, IL-8, and IL-10. Only small changes in these cytokines were observed. The levels of these cytokines generally was low at intake and within ranges expected for healthy subjects.

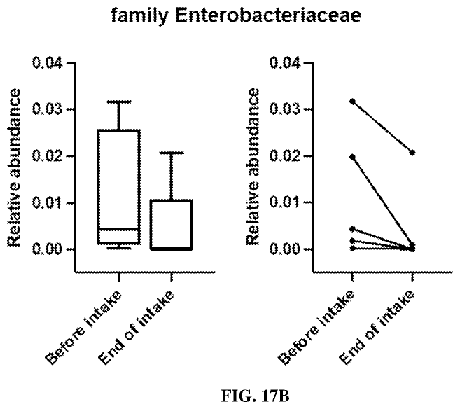

Metagenomic relative abundance of genus Parabacteroides (members of commensal taxa) and family Enterobacteriaceae (members of pathobiont taxa) were examined in five patients at screening and at the end of the intake period. The relative abundance of Parabacteroides increased in four out of the five patients (FIG. 17A), and the relative abundance of Enterobacteriaceae decreased in all five participants (FIG. 17B) following treatment with the selected oligosaccharide composition, assessed in fecal samples collected from the subjects. These data demonstrate that the selected oligosaccharide composition is capable of altering the gut microbiome of human patients with ulcerative colitis. Specifically, these data demonstrate that the selected oligosaccharide composition increased the abundance of commensal taxa (e.g., Parabacteroides) relative to pathobionts (e.g., Enterobacteriaceae) in human patients with ulcerative colitis.

Metagenomics were also utilized to demonstrate that administration of the selected oligosaccharide causes a reduction in adherent-invasive E. coli pathobionts. To identify virulence signatures associated with adherent-invasive E. coli, Pangenome-based Phylogenomic Analysis (PanPhlAn) was used to quantify the pangenome of E. coli genes using a pangenome reference of 90% amino acid identity gene clusters. Abundances of gene references were aggregated to the KEGG KO gene family classification level. This analysis demonstrated that three gene annotations that are associated with adherent-invasive E. coli isolates (fimH, ompA, and ompC) were decreased after intake of the selected oligosaccharide composition. These data demonstrate that the selected oligosaccharide composition is capable of reducing the abundance of adherent-invasive E. coli in gut microbiomes of human patients with ulcerative colitis to. This is significant because adherent-invasive E. coli have been implicated in the pathogenesis of ulcerative colitis.

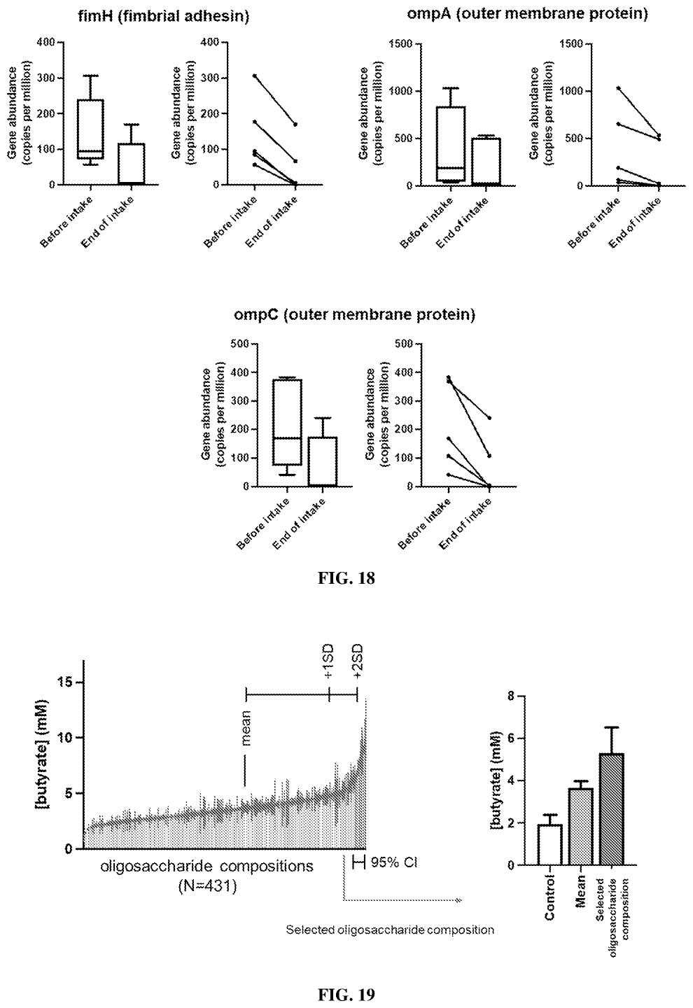

The data suggest that the selected oligosaccharide is capable of affecting several pathways/mechanisms involved in inflammatory diseases such as inflammatory bowel diseases, e.g., UC and CD (see, e.g., FIG. 9). The data suggest that the selected oligosaccharide (a) increases SCFAs (e.g., butyrate) (FIGS. 1, 2 10 and 19), (b) increases the abundance of commensals (e.g., Parabacteroides) and decreases the abundance of pathobionts (e.g., Enterobacteriaceae) (FIGS. 3, 12, 13, 14, and 17), and E. coli (FIG. 18), (c) reduces intestinal inflammation (e.g., fecal calprotectin and lactoferrin) (FIG. 15), and (d) improves quality of life (QoL, e.g. SCCAI score) (FIG. 16). Collectively, the data suggest that the selected oligosaccharide compositions described herein are useful in treating patients exhibiting an inflammatory disease, such as inflammatory bowel diseases, e.g., UC and CD.

### Example 12. Production of a Selected Oligosaccharide Composition at 25 kg Scale from Galactose Using a Soluble Acid Catalyst

A procedure was developed for the synthesis of a selected oligosaccharide composition as described in Example 1 at a 25 kilogram scale. 25 kg of anhydrous galactose, 0.38 kg citric acid anhydrous acid catalyst and 6.5 kg water were added to a reaction vessel (oil-jacketed 50 L continuous stirred tank reactor (CSTR)) equipped with a distillation condenser unit. The contents were agitated at approximately 120 rpm and the vessel temperature was increased over a 2-4.0 hour period to about 130° C. at atmospheric pressure. The mixture was maintained at temperature for an additional 3-5 hours, after which the heating was stopped and a crash-cooling procedure was initiated. After 30 minutes of cooling, 6 L of pre-heated water (quench water) was quickly added to the reaction mixture at a rate of 500 mL/min. Once all quench water was added, the reactor contents were allowed to mix until completely dissolved and cooled to an internal temperature at or below 55° C. The reaction mixture was drained from the vessel, resulting in approximately 45-50 kg of crude oligosaccharide as an aqueous solution (approximately 47-55 wt %).

The oligosaccharide composition was purified by flowing through a 0.45 micron filter to obtain approximately 45-50 kg of filtered composition.

### Example 13. Production of a Selected Oligosaccharide Composition at 500 kg Scale from Galactose Using a Soluble Acid Catalyst

A procedure was developed for the synthesis of a selected oligosaccharide composition as described in Example 1 at a 500 kilogram (2000 L) scale. 125 kg of water was first added to a clean glass-lined 2000 L continuous stirred tank reactor. The water was agitated at approximately 60 rpm and the jacket of the reactor was heated to 85° C. 500 kg of anhydrous galactose and 7.5 kg citric acid anhydrous acid catalyst were then added to the reactor.

The contents were agitated at approximately 60 rpm and the vessel temperature was increased over a 2-4.0 hour period to about 130° C. at atmospheric pressure. The mixture was maintained at temperature for at least an additional 5 hours, after which the heating was stopped and a cooling procedure to reduce the temperature of the contents to 25° C. or less within hours was initiated. The temperature of the jacket of the reactor was reduced, and approximately 190 kg hot water (65° C.) was added to the contents of the reactor over about ten minutes. The temperature of the jacket of the reactor was then reduced further, an additional approximately 310 kg of room temperature water was added to the contents of the reactor, and the contents were allowed to cool to room temperature.

The oligosaccharide composition was subsequently purified by flowing through a 0.45 micron filter.

### Example 14. Spray Drying Procedure

Individual batches of the selected oligosaccharide composition, as produced in Example 13 were spray-dried. The chamber of an SPX Anhydro MicraSpray 400 instrument equipped with a rotary atomizer was inerted to bring the oxygen concentration inside the chamber to <1%. The drying gas flow was started and set to 440 kg/hr. Once the drying gas flow was stable, the spray drying instrument was set to the following setpoints: (i) Spray dryer pressure setpoint=1050 mbar; (ii) Spray dryer chamber skin heater=60° C.; (iii) Recirculation fan pre-heater=20° C.; (iv) Steam heater inlet=90° C.; (v) Rotary atomizer speed=25000 RPM; (vi) Cyclone skin heater=90° C.; and (vii) Baghouse skin heater=40° C. Once the above spray dryer process parameters had stabilized, the blank solution (Purified Water) was turned on and set to 7.5 kg/hr., and the steam heater inlet temperature was increased to 145° C.

After the spray dryer outlet temperature had stabilized to around 90° C., the solvent feed was changed from blank solution (Purified Water) to feed solution (the selected oligosaccharide composition), and the feed rate was adjusted to 15.0 kg/hr. The spray-dried oligosaccharide composition was collected in an 800 L conical vessel as it was dried by the instrument.

### Example 15. HSQC NMR Analysis of Large-Scale Batches of the Selected Oligosaccharide Composition Using a Bruker NMR Machine

A determination of HSQC NMR spectra of samples of the selected oligosaccharide composition described in Example 1, and produced as described in Examples 12 and 13, was obtained using a Bruker NMR machine and analyzed using the MNova software package from Mestrelab Research, according to the HSQC NMR methods described in Example 8.

### Results

A single batch of the selected oligosaccharide composition produced according to the process described in Example 12 (25 kg galactose; 50 L scale) and four batches of the selected oligosaccharide composition produced according to the process described in Example 13 (500 kg galactose; 2000 L scale) (having an average DP of 12.7 (+0.4)) were analyzed using the HSQC NMR methods described in Example 8. Prior to HSQC NMR analysis, samples were de-monomerized using the ethanol precipitation procedure described in Example 5.

The relative size of each of the peaks (i.e., integration of peaks/signals 1-11 using the predefined integral regions of Table 9 with elliptical shapes; herein referred to as area under the curve (AUC)) collected for the NMR spectra of the analyzed batches of the selected oligosaccharide composition was determined, as shown below in Table 15:

TABLE 15
HSQC NMR data (batches produced using processes as described in Examples 12 and 13)

| | | | 50 L | 2000 L | 2000 L | 2000 L | 2000 L |
|---|---|---|---|---|---|---|---|
| | | | batch | batch #1 | batch #2 | batch #3 | batch #4 |
| | Center | | (% of total | (% of total | (% of total | (% of total | (% of total |
| | Position (ppm) | | areas of | areas of | areas of | areas of | areas of |
| Signal | ¹H | ¹³C | signals 1-11) | signals 1-11) | signals 1-11) | signals 1-11) | signals 1-11) |
| 1 | 5.04 | 108.38 | 4.09 | 4.35 | 4.24 | 4.23 | 4.32 |
| 2 | 5.02 | 101.68 | 1.18 | 0.82 | 1.09 | 1.16 | 1.15 |
| 3 | 4.98 | 98.99 | 15.42 | 18.60 | 19.12 | 15.97 | 15.43 |
| 4 | 4.81 | 101.2 | 0.91 | 1.27 | 1.00 | 1.25 | 1.22 |
| 5 | 4.6 | 104.96 | 2.01 | 2.40 | 2.51 | 1.94 | 1.96 |
| 6 | 4.45 | 103.84 | 10.51 | 11.44 | 11.20 | 10.58 | 10.83 |
| 7 | 4.19 | 82.1 | 4.85 | 4.66 | 4.60 | 4.27 | 4.47 |
| 8 | 4.14 | 76.91 | 12.53 | 10.16 | 11.06 | 11.60 | 11.47 |
| 9 | 4.11 | 81.72 | 16.30 | 15.04 | 14.41 | 16.15 | 16.19 |
| 10 | 4.06 | 77.25 | 21.97 | 21.46 | 21.36 | 22.18 | 22.01 |
| 11 | 3.98 | 83.52 | 10.26 | 9.80 | 9.41 | 10.67 | 10.96 |

These HSQC NMR data for large-scale batches of the selected oligosaccharide composition (produced using the processes described in Examples 12 and 13) provide substantially similar relative integrations of the peaks/signals 1-11 using the predefined integral regions as demonstrated for the batches of the selected oligosaccharide composition produced using the processes described in Examples 2-4. Thus, these data demonstrate that producing the selected oligosaccharide composition at large-scale (e.g., 500 kg galactose; 2000 L scale) does not significantly change the chemical properties of the selected oligosaccharide composition.

One of the 2000 L batches (2000 L batch #1) analyzed above was further subjected to the spray drying procedure described in Example 14. Following the spray drying procedure, a sample of the spray dried composition was prepared for HSQC NMR analysis by dissolving 30 mg of the spray dried composition (after de-monomerization using the ethanol precipitation procedure described in Example 5) in 300 μL of D2O with 0.1% acetone as internal standard. The solution was then placed into a 3 mm NMR tube and using the HSQC NMR methods described in Example 8. The relative size of each of the peaks (i.e., integration of peaks/signals 1-11 using the predefined integral regions of Table 9 with elliptical shapes; herein referred to as area under the curve (AUC)) collected for the NMR spectra of the analyzed batches of the selected oligosaccharide composition was determined and compared to the NMR spectrum of 2000 L batch #1 prior to spray drying, as shown below in Table 16:

TABLE 16
HSQC NMR data (before and after spray drying)

| | | | 2000 L batch #1 | 2000 L batch #1 |
|---|---|---|---|---|
| | Center Position | | (before spray drying) | (after spray drying) |
| | (ppm) | | (% of total areas | (% of total areas |
| Signal | ¹H | ¹³C | of signals 1-11) | of signals 1-11) |
| 1 | 5.04 | 108.38 | 4.35 | 4.20 |
| 2 | 5.02 | 101.68 | 0.82 | 0.95 |
| 3 | 4.98 | 98.99 | 18.60 | 18.35 |
| 4 | 4.81 | 101.2 | 1.27 | 1.29 |
| 5 | 4.6 | 104.96 | 2.40 | 2.15 |
| 6 | 4.45 | 103.84 | 11.44 | 11.22 |
| 7 | 4.19 | 82.1 | 4.66 | 4.77 |
| 8 | 4.14 | 76.91 | 10.16 | 10.53 |
| 9 | 4.11 | 81.72 | 15.04 | 15.41 |
| 10 | 4.06 | 77.25 | 21.46 | 21.60 |
| 11 | 3.98 | 83.52 | 9.80 | 9.52 |

The HSQC NMR spectrum of the 2000 L batch #1 before and after spray drying are substantially similar to one another, with only minor variations in the relative size of each of the peaks (i.e., integration of peaks/signals 1-11 using the predefined integral regions of Table 9 with elliptical shapes; herein referred to as area under the curve (AUC)). Thus, these HSQC NMR data demonstrate that spray drying has little-to-no effect on the HSQC NMR spectrum and chemical properties of the selected oligosaccharide composition.

### Example 16. Determination of Glycosidic Bond Distribution of Large-Scale Batches of the Selected Oligosaccharide Composition Using Permethylation Analysis

A determination of glycosidic bond distribution of samples of the selected oligosaccharide composition described in Example 1, and produced as described in Examples 12 and 13, was performed using permethylation analysis, according to the methods described in Example 9. Samples were demonomerized prior to permethylation analysis. A single batch of the selected oligosaccharide composition produced according to the process described in Example 12 (25 kg galactose; 50 L scale) and four batches of the selected oligosaccharide composition produced according to the process described in Example 13 (500 kg galactose; 2000 L scale) (having an average DP of 12.7 (±0.4)) were tested in this Example.

### Results

Permethylation data was collected and each batch was analyzed with three technical replicates. Averaged data with standard deviations (std. dev.) among the three technical replicates relating to the radicals present in these batches of de-monomerized oligosaccharide composition are provided below in Table 17:

TABLE 17
Permethylation data (de-monomerized oligosaccharide composition
produced by the processes described in Examples 12 and 13)

| | 2000 L batch #1 | | 2000 L batch #2 | | 2000 L batch #3 | | 2000 L batch #4 | | 50 L batch | |
|---|---|---|---|---|---|---|---|---|---|---|
| | Mean | Std. | Mean | Std. | Mean | Std. | Mean | Std. | Mean | Std. |
| Radicals | (N = 3) | dev. | (N = 3) | dev. | (N = 3) | dev. | (N = 3) | dev. | (N = 3) | dev. |
| t-galactofuranose | 6.95% | 0.35% | 7.17% | 0.09% | 9.03% | 0.51% | 9.02% | 0.76% | 8.43% | 0.32% |
| monoradicals | | | | | | | | | | |
| t-galactopyranose | 29.42% | 1.75% | 30.10% | 1.17% | 29.17% | 0.38% | 28.47% | 0.90% | 28.56% | 0.83% |
| monoradicals | | | | | | | | | | |
| 2-galactofuranose | 2.28% | 0.28% | 2.20% | 0.08% | 2.45% | 0.21% | 2.54% | 0.21% | 2.28% | 0.28% |
| and/or 2- | | | | | | | | | | |
| glucofuranose | | | | | | | | | | |
| monoradicals | | | | | | | | | | |
| 3-galactopyranose | 6.43% | 0.21% | 6.28% | 0.36% | 6.17% | 0.25% | 6.36% | 0.43% | 6.33% | 0.33% |
| monoradicals | | | | | | | | | | |
| 3-galactofuranose | 3.38% | 0.31% | 3.18% | 0.08% | 3.49% | 0.16% | 3.66% | 0.14% | 3.37% | 0.22% |
| monoradicals | | | | | | | | | | |
| 2-galactopyranose | 3.93% | 0.13% | 3.89% | 0.21% | 3.89% | 0.11% | 4.01% | 0.13% | 3.98% | 0.15% |
| monoradicals | | | | | | | | | | |
| 4-galactopyranose | 5.53% | 0.19% | 5.43% | 0.28% | 5.62% | 0.21% | 5.80% | 0.30% | 5.70% | 0.26% |
| and/or 5- | | | | | | | | | | |
| galactofuranose | | | | | | | | | | |
| monoradicals | | | | | | | | | | |
| 2,3-galactofuranose | 0.00% | 0.00% | 0.00% | 0.00% | 0.00% | 0.00% | 0.00% | 0.00% | 0.00% | 0.00% |
| diradicals | | | | | | | | | | |
| 6-galactofuranose | 5.41% | 0.46% | 5.57% | 0.18% | 5.92% | 0.37% | 5.92% | 0.40% | 5.39% | 0.39% |
| monoradicals | | | | | | | | | | |
| 6-galactopyranose | 18.53% | 0.81% | 18.52% | 0.27% | 16.67% | 0.25% | 16.48% | 0.11% | 17.31% | 0.11% |
| monoradicals | | | | | | | | | | |
| 3,4-galactopyranose | 2.15% | 0.15% | 2.08% | 0.15% | 2.18% | 0.10% | 2.16% | 0.11% | 2.16% | 0.06% |
| and/or 3,5- | | | | | | | | | | |
| galactofuranose | | | | | | | | | | |
| and/or 2,3- | | | | | | | | | | |
| galactopyranose | | | | | | | | | | |
| diradicals | | | | | | | | | | |
| 2,4-glucopyranose | 0.86% | 0.06% | 0.84% | 0.03% | 0.89% | 0.03% | 0.93% | 0.06% | 0.94% | 0.03% |
| and/or 2,5- | | | | | | | | | | |
| glucofuranose | | | | | | | | | | |
| and/or 2,4- | | | | | | | | | | |
| galactopyranose | | | | | | | | | | |
| and/or 2,5- | | | | | | | | | | |
| galactofuranose | | | | | | | | | | |
| diradicals | | | | | | | | | | |
| 2,3,4-galactopyranose | 0.16% | 0.14% | 0.20% | 0.17% | 0.10% | 0.17% | 0.08% | 0.14% | 0.26% | 0.02% |
| and/or 2,3,5- | | | | | | | | | | |
| galactofuranose | | | | | | | | | | |
| triradicals | | | | | | | | | | |
| 3,6-galactofuranose | 1.52% | 0.19% | 1.41% | 0.10% | 1.54% | 0.18% | 1.54% | 0.24% | 1.59% | 0.20% |
| diradicals | | | | | | | | | | |
| 4,6-galactopyranose | 4.69% | 0.18% | 4.54% | 0.28% | 4.59% | 0.13% | 4.66% | 0.16% | 4.79% | 0.20% |
| and/or 5,6- | | | | | | | | | | |
| galactofuranose | | | | | | | | | | |
| diradicals | | | | | | | | | | |
| 3,6-galactopyranose | 4.27% | 0.25% | 4.19% | 0.13% | 4.07% | 0.04% | 4.11% | 0.12% | 4.34% | 0.10% |
| and/or 2,6- | | | | | | | | | | |
| galactofuranose | | | | | | | | | | |
| diradicals | | | | | | | | | | |
| 2,6-galactopyranose | 2.01% | 0.10% | 2.02% | 0.11% | 1.94% | 0.02% | 1.99% | 0.03% | 2.13% | 0.08% |
| diradicals | | | | | | | | | | |
| 3,4,6-galactopyranose | 1.23% | 0.13% | 1.16% | 0.10% | 1.23% | 0.11% | 1.21% | 0.08% | 1.31% | 0.03% |
| and/or 3,5,6- | | | | | | | | | | |
| galactofuranose | | | | | | | | | | |
| and/or 2,3,6- | | | | | | | | | | |
| galactofuranose | | | | | | | | | | |
| triradicals | | | | | | | | | | |
| 2,3,6-galactopyranose | 1.22% | 0.22% | 1.18% | 0.25% | 1.05% | 0.05% | 1.06% | 0.08% | 1.15% | 0.03% |
| and/or 2,4,6- | | | | | | | | | | |
| galactopyranose | | | | | | | | | | |
| and/or 2,5,6- | | | | | | | | | | |
| galactofuranose | | | | | | | | | | |
| triradicals | | | | | | | | | | |
| 2,3,4,6-galactopyranose | 0.03% | 0.06% | 0.04% | 0.07% | 0.00% | 0.00% | 0.00% | 0.00% | 0.00% | 0.00% |
| and/or 2,3,5,6- | | | | | | | | | | |
| galactofuranose | | | | | | | | | | |
| quadradicals | | | | | | | | | | |

### Example 17. Assignment of HSQC NMR Peaks/Signals in Spectrum of the Selected Oligosaccharide Composition

A determination of HSQC NMR spectra of the selected oligosaccharide compositions described in Example 1, and produced as described in Examples 12 and 13, was performed using a Bruker NMR machine, according to the protocol described below, in order to assign the specific peaks/signals to be annotated as belonging to discrete bond types present within the selected oligosaccharide composition.

### Method

### Sample Preparation:

30 mg of a previously lyophilized solid sample of the oligosaccharide composition was dissolved in 300 μL of D₂₀ with 0.1% acetone as internal standard. The solution was then placed into a 3 mm NMR tube.

### NMR Experiment:

Each sample was analyzed in a Bruker NMR operating at 600 MHz equipped with a CP QCI probe with 1H/19F, 13C, 15N, 31P.

Each sample was subjected to a phase-sensitive, multiplicity-edited gradient-enhanced ¹H-¹³C heteronuclear single quantum coherence (HSQC) experiment using PEP and inversion, refocusing and matched sweep adiabatic pulses with gradients in back-inept using the following pulse sequence diagram:

### Spectral Analysis and Results

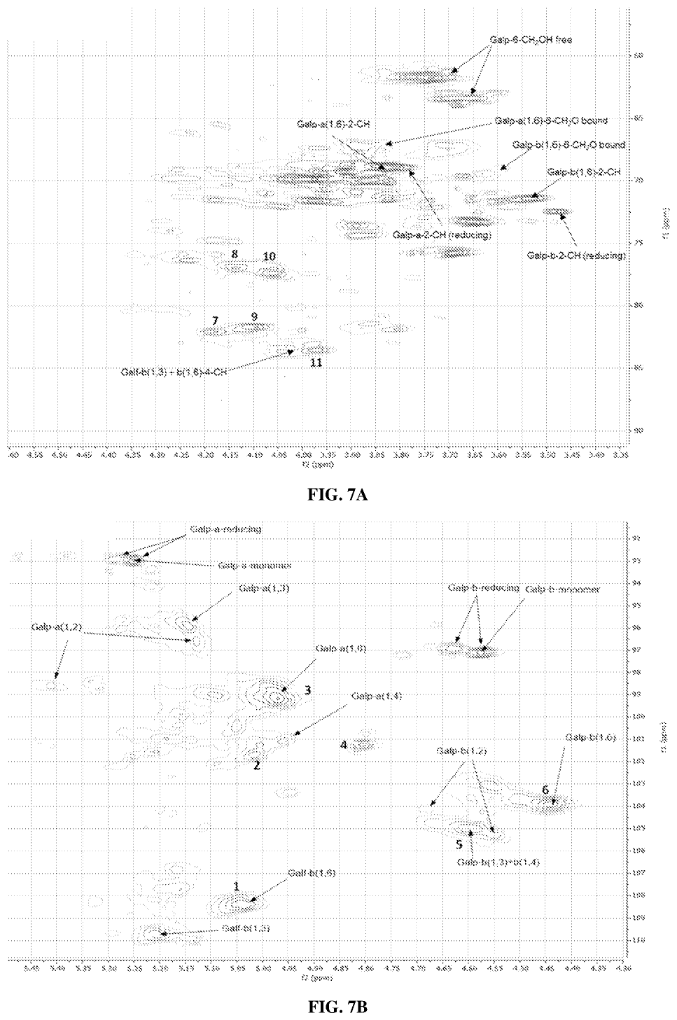

The resulting spectra were analyzed using the MNova software package from Mestrelab Research (Santiago de Compostela, Spain). The spectra were referenced to the internal acetone signal (1H-2.22 ppm; ¹³C-30.8 ppm) and phased using the Regions2D method in both the F2 and F1 dimension. Apodization using 90 degree shifted sine was applied in both the F2 and F1 dimension. Assignments of peaks/signals as belonging to discrete bond types present within the selected oligosaccharide were based on strategies like the “1H NMR structural-reporter-group concept” (Leeuwen et al. Carbohydrate Research 343 (2008), 1114-1119), literature chemical shifts, H2BC, and HMBC correlations. Dimers used in the analysis included (Carbosynth, Inc): Galp-b (1-2)-Galp, Galp-a (1-2)-Galp, Galp-a (1-3)-Galp, Galp-b (1-3)-Galp, Galp-b (1-4)-Galp, Galp-a (1-4)-Galp, Galp-b (1-6)-Galp, and Galp-a (1-6)-Galp. Linkage abundances were compared with permethylation data and cross referenced with literature values and other oligosaccharide compositions. Assignments are provided in FIGS. 7A-7B.

### Example 18. Stability of the Selected Oligosaccharide Composition

The stability of the selected oligosaccharide composition was assessed by incubating the selected oligosaccharide composition at elevated temperatures for a period of time. 150 mL aliquots of samples of the selected oligosaccharide composition (50% w;v) were brought to target temperatures (70° C., 80° C., 90° C. or 100° C.) in oil-jacketed 250 mL reactors and then sampled periodically over a period of 6 hours (10=point at which target temp reached). Samples were run on SEC using the methods described in Example 6 to determine Mw, Mn, and percent DP2+ at the various time points. FIG. 8 shows the Mw, Mn, and percent DP2+ of samples of the selected oligosaccharide composition at the endpoint of the incubation (i.e., time=6 hours). The structural integrity of the selected oligosaccharide composition is minimally affected by exposure to temperatures of 70° C. and 80° C. in the presence of water for extended periods of time (i.e., six hours). However, exposure to higher temperatures in the presence of water can lead to hydrolysis and a shortening of the average lengths of oligosaccharides comprised in the selected oligosaccharide composition as measured by Mw, Mn, and % DP2+.

This suggests that the chemical structure of the selected oligosaccharide composition is minimally affected by being held at elevated temperatures of 70° C. and 80° C. for extended periods of time (i.e., six hours). Furthermore, these data suggest that post-processing steps (e.g., quenching) after the production of the selected oligosaccharide composition (e.g., production at large-scale, e.g., using Examples 12 and 13) should be performed in a timely manner in order to maintain the structural integrity of the selected oligosaccharide composition with these data taken into account. For example, quenching of a reaction mixture following the production of the selected oligosaccharide composition in order to cool the selected oligosaccharide composition should be done in a timely manner, in accordance with this Example.

### Example 19. The Selected Oligosaccharide Composition Increases Production of SCFA in Fecal Samples from Healthy Subjects in an Ex Vivo Culture System

Fecal samples from ten healthy subjects were incubated anaerobically in Clostridium minimal medium (Theriot et al, 2014, Nature Communications), supplemented with 0.75 mM urea and 0.1% (w/v) trypticase peptone, without (negative control) or with the selected oligosaccharide composition at a final concentration of 0.5% (w/v) for 45 hours at 37° C. Each fecal sample incubation was performed with three biological replicates. After incubation, samples were sedimented by centrifugation and culture supernatants were collected. SCFA concentrations were quantified in culture supernatants using gas chromatography with flame-ionization detection (GC-FID).

The mean SCFA values across sample replicates were included in the box-whisker plots and p-values were determined using two-tailed, paired t-tests (FIG. 10). The selected oligosaccharide composition increased the production of SCFA across the ten fecal samples to median concentration of 47.0 mM compared to 15.2 mM with the negative control. Furthermore, the selected oligosaccharide composition increased each of the three SCFAs (i.e. acetate, propionate, and butyrate) in each of the ten fecal samples tested. Incubation with the selected oligosaccharide composition produced median values of 27.8 mM acetate, 15.0 mM propionate and 6.1 mM butyrate compared to the median values of 10.0 mM acetate, 3.1 mM propionate, and 2.1 mM butyrate with the negative control (water).

Genomic DNA concentrations were also determined using the Quant-iT pricoGeen dsDNA assay kit (Invitrogen). The genomic DNA was subjected to quantitative PCR with oligonucleotide primers targeting the bacterial 16S rRNA gene. The concentration of genomic DNA extracted and the number of copies of the 16S rRNA gene per unit volume of culture are measurements of the abundance of bacteria in each culture. The median genomic DNA concentration across fecal samples incubated with the selected oligosaccharide composition was 30.2 ng/μL compared to 2.7 ng/μL with the negative control. Similarly, the median 16S rRNA gene copies from fecal samples incubated with the selected oligosaccharide composition was 1.1×108 copies/μL compared to 2.2×107 copies/μL for the negative control. The differences between the selected oligosaccharide composition and the negative control for both genomic DNA concentrations and 16S rRNA gene copies were statistically significant determined by two-tailed, paired t-tests (p-value <0.0001).

These results demonstrate that the selected oligosaccharide composition is consistently fermented (consumed) by fecal microbiota from different healthy subjects. This fermentation yields a balanced SCFA production profile, suggesting that the selected oligosaccharide composition supports the robust growth and metabolism of a diverse set of commensal gut bacteria that produce SCFAs such as acetate, propionate, and butyrate.

### Example 20. The Selected Oligosaccharide Composition Modulates the Fecal Microbiome in Fecal Samples from Healthy Subjects in an Ex Vivo Culture System

To understand the shifts in taxonomic composition produced by incubation with the selected oligosaccharide composition, fecal microbiomes were characterized by shotgun metagenomic sequencing (Diversigen, MN USA). Shotgun metagenomic sequencing libraries were prepared using the NexteraXT kit before sequencing on the Illumina NextSeq platform to at least 10 million reads per library. Taxa count tables from shotgun metagenomic sequencing data were generated by the SHOGUN pipeline using a database including the first 20 strains per species in RefSeq v87. Bray-Curtis dissimilarity were computed on the genus level taxa tables between all samples. In a non-metric multi-dimensional scaling (NMDS) ordination plot, fecal samples incubated with the selected oligosaccharide composition form a cluster representing a distinct microbiome composition from control samples (FIG. 11). This is quantified as a mean Bray-Curtis shift of 0.53 (standard deviation of 0.06) versus a more coherent cluster within controls (mean of 0.35 with a standard deviation of 0.075) and within samples incubated with the selected oligosaccharide composition (mean of 0.299 with a standard deviation of 0.123) at 95% Confidence Interval. These data suggest that the selected oligosaccharide composition might produce a consistent microbiome composition profile even when incubated in subjects or samples having inconsistent input microbiome composition profiles (e.g., prior to administration of the selected oligosaccharide composition).

### Example 21. The Selected Oligosaccharide Composition Produces Taxonomic Changes in Individual Fecal Samples after Incubation with the Selected Oligosaccharide Composition

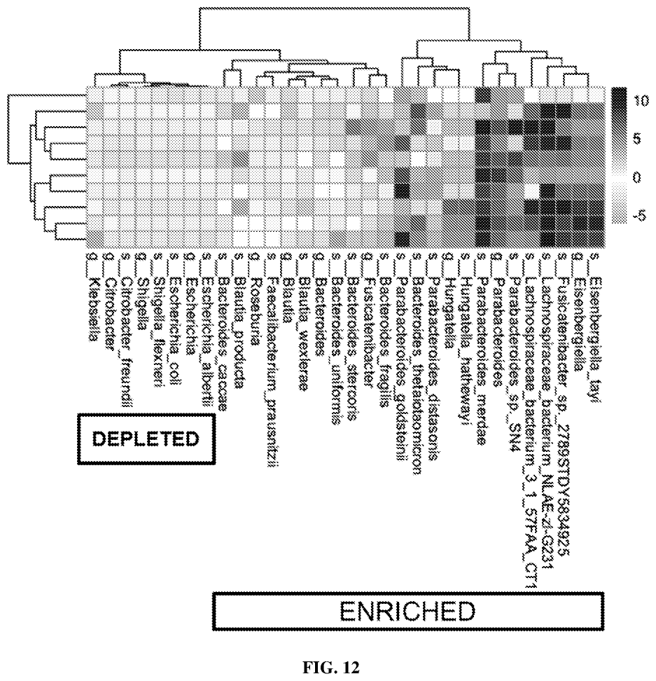

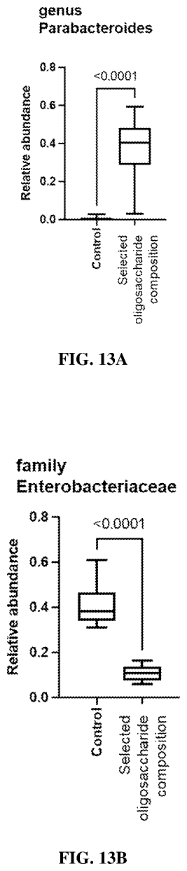

To identify genera and species that were modulated (e.g., increased or decreased in relative or absolute abundance) by incubation with the selected oligosaccharide composition, microbiome genera and species taxa tables were filtered for a mean of 0.1% and a log 2-fold ratio of incubation with the selected oligosaccharide composition relative to control. This produced a set of commensal taxa that were consistently enriched in healthy subjects (e.g. Parabacteroides, Eisenbergiella), and a set of pathobionts the were consistently depleted (e.g. Escherichia, Klebsiella, Shigella, Citrobacter) (FIG. 12). Despite natural differences in microbiome composition, the selected oligosaccharide composition was able to selectively and consistently enrich a set of commensal bacteria and consistently deplete pathobionts. Specifically, the selected oligosaccharide composition enriched taxa belonging to the genus Parabacteroides. The median relative abundance of the genus Parabacteroides across ten fecal samples of healthy subjects increased from 0.2% with the negative control (water) to 40.4% with the selected oligosaccharide composition (FIG. 13A). The genus Parabacteroides belongs to the phylum Bacteroides which consists of bacteria that encode wide array of glycan utilization systems and typically produce propionate as a byproduct of glycan fermentation (Reichardt et al, The ISME Journal volume 8, pages 1323-1335 (2014)). The genus Parabacteroides was also shown to be associated with remission in UC patients after fecal microbiota transplantation (Paramsothy et al, Lancet. 2017 Mar. 25; 389 (10075): 1218-1228.). In addition to members of the genus Parabacteroides, the selected oligosaccharide composition also enriched particular taxa of the phylum Firmicutes. The selected oligosaccharide composition increased the relative abundance of the genera Eisenbergiella and Fusicatenibacter, both of which belong to family Lachnospiraceae. This family contains a number of species that produce butyrate as a byproduct of glycan fermentation, including the species Eisenbergiella tayi (which was enriched by the selected oligosaccharide composition). The selected oligosaccharide composition also enriched two unclassified species in the family Lachnospiraceae, as well as an unclassified species in the genus Eubacterium.

These enrichments are noteworthy because members of the phylum Bacteroidetes and family Lachnospiracede were shown to be depleted in fecal samples of IBD patients compared to healthy non-IBD controls (Frank et al, PNAS Aug. 21, 2007 104 (34) 13780-13785). Furthermore, butrate and propionate produced by these groups of bacteria provide a sources of energy for colonic epithelial cells, support epithelial integrity and function, and modulate the intestinal immune response to reduce colitis (Pryde et al, FEMS Microbiol Lett. 2002 Dec. 17; 217 (2): 133-9.; Smith et al, SCIENCE, 1 Feb. 2013, Vol 339, Issue 6119, pp. 548-554), Arpaia et al, Nature. 2013 Dec. 19; 504 (7480): 451-5. doi: 10.1038/nature12726. Epub 2013 Nov. 13.). Specifically, the selected oligosaccharide composition depleted taxa belonging to the family Enterobacteriaceae, which is known to harbor several pathiobiont taxa. The median relative abundance of the family Enterobacteriaceae across the ten fecal samples decreased from 38.2% with the negative control (water) to 10.8% with the selected oligosaccharide composition (FIG. 13B). The selected oligosaccharide composition also decreased the relative abundance of several genera in the family Enterobacteriaceae such as the genera Escherichia, Shigella, Salmonella, and Citrobacter. These data show that the selected oligosaccharide composition can deplete numerous different pathiobiont taxa that belong to the family Enterobacteriaceae, a taxonomic group associated with disease in patients with ulcerative colitis (Caruso et al, Nat Rev Immunol. 2020 July; 20 (7): 411-426. doi: 10.1038/s41577-019-0268-7.).

### Example 22. The Selected Oligosaccharide Composition Selectively Supported the Growth and Abundance of Commensal Bacteria in Monoculture and Did not Support the Growth of Pathobionts

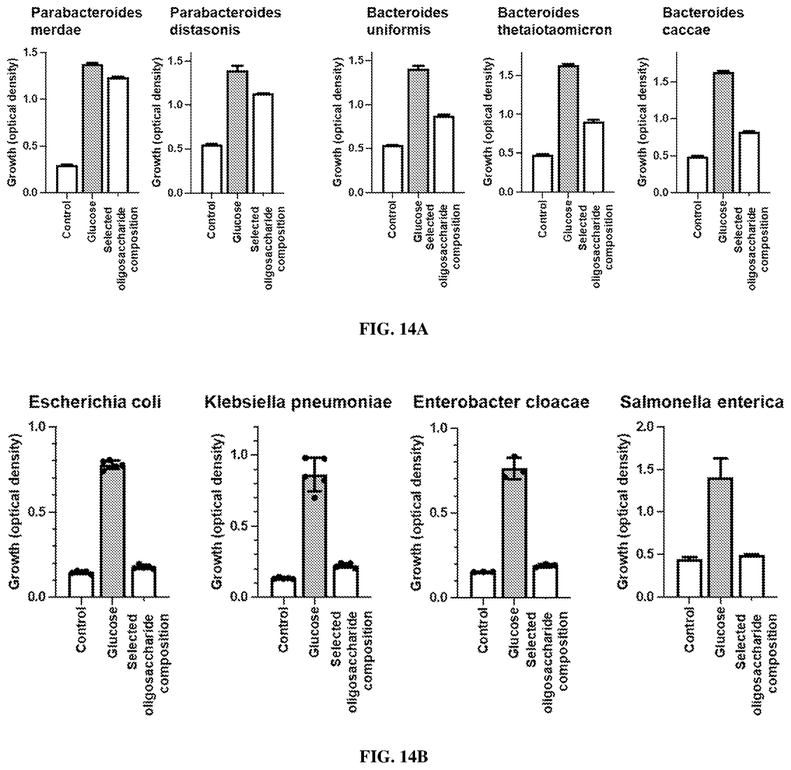

The taxomonic enrichments and depletions observed after ex vivo incubation of fecal samples suggested that certain commensal bacteria harbor the ability to utilize the selected oligosaccharide composition as a substrate for growth while pathionts lack that ability. Selected commensal and pathobiont species were incubated anaerobically in culture medium without (negative control) or with 0.5% w/v the selected oligosaccharide composition or with glucose (positive control). The optical density of each culture was measured every 15 minutes for 24-48 hours using a BioTek Powerwave spectrophotometer. Each culture was performed with three biological replicates. The mean maximum optical density values were determined across culture replicates and included in the bar plots. It was found that the selected oligosaccharide composition supported strong growth of two different Parabacteroides species (Parabacteroides merdae and Parabacteroides distansonis), relative to negative control, indicating that these species were capable of utilizing the selected oligosaccharide composition for fermentation (FIG. 14A). The selected oligosaccharide composition also supported levels of growth of three different Bacteroides species (Bacteroides uniformis, Bacteroides thetaiotaomicron, and Bacteroides caccae), relative to negative control, demonstrating that these species also able to use the selected oligosaccharide composition as a growth substrate. Conversely, no growth was observed with the pathobionts species Escherichia coli, Klebsiella pneumoniae, Enterobacter cloacae, and Salmonella enterica, relative to negative control (FIG. 14B).

Five different strains of the species Escherichia coli and Klebsiella pneumoniae were tested, as well three different strains of Enterobacter cloacae. One strain of each of the other species were tested. Individual points shown in the pathobiont bar graphs represent the mean value for individual strains of the species tested.

These data show that commensal bacteria in the genera Parabacteroides and Bacteroides are able to utilize the selected oligosaccharide composition as a growth substrate, but pathobiont species in the family Enterobacteriaceae lack that ability. Together with ex vivo metagenomic sequencing data, these monoculture data indicate that the selected oligosaccharide composition can selectively support the growth of commensal gut bacteria and give them an ecological advantage over pathobionts that colonize the gut in patients suffering from ulcerative colitis.

### Example 23. Mesalamine does not Influence Ex Vivo Fermentation of the Selected Oligosaccharide Composition

5-aminosalicilic acids (5-ASA), such as mesalamine, are standard-of-care compounds for the treatment of patients with mild-to-moderate ulcerative colitis. The ability of the selected oligosaccharide composition to support the growth of microbial taxa in the presence of mesaline was determined. Fecal samples from ten healthy subjects were incubated anaerobically in supplemented Clostridium minimal medium without (negative control) or with the selected oligosaccharide composition. Samples with the selected oligosaccharide composition were also incubated with 0.0, 0.5, 2, 8, 32, 125, or 500 μM mesalamine. Each fecal sample incubation was performed with three biological replicates. After incubation, samples were sedimented by centrifugation and culture supernatants were collected for SCFA quantification by GC-FID.

As previously observed, incubation with the selected oligosaccharide composition increased the concentration of total SCFA and each of acetate, butyrate, and propionate individually compared to the negative control (water). Importantly, none of the samples incubated with mesalamine showed any significant differences in SCFA production by the selected oligosaccharide composition.

To understand if mesalamine influenced understand the shifts in taxonomic composition produced by incubation with the selected oligosaccharide composition, the fecal samples were characterized by 16S rRNA amplicon seuquencing. Genomic DNA was extracted from fecal samples using DNeasy PowerSoil extraction plates (Qiagen) and quantified using the Quant-iT PicoGreen dsDNA assay (Invitrogen). 16S rRNA libraries were prepared by PCR amplification with the 515F/806R primer set before sequencing on the Illumina NextSeq platform to at least 25 thousand reads. Sequences of 16S rRNA genes were analyzed by UNOISE clustering and denoising of raw sequences followed by DADA2/RDP taxonomic calling. 16S rRNA sequencing of samples from the ten fecal samples were compared using Bray-Curtis dissimilarity of samples incubated with the selected oligosaccharide composition and without mesalamine. All 5-ASA concentrations tested had mean Bray-Curtis dissimilarity (0.06 (S.D. 0.02)) resembling replicate sequencing dissimilarity (0.06 (S.D. 0.03)), suggesting that the presence of mesalamine had a very low effect on the fecal microbiome composition produced by the selected oligosaccharide composition. As a comparison, the variation within the samples incubated with the selected oligosaccharide composition and without mesalamine was 0.31 (S.D. 0.05) and dissimilarity to negative controls was 0.46 (S.D. 0.03)). Together, these results indicated that mesalamine does not interfere with ex vivo fermentation of the selected oligosaccharide composition in fecal samples from healthy subjects.

### Example 24. Clinical Trial to Assess Ability of the Selected Oligosaccharide Composition to Treat Patients with Ulcerative Colitis Disease

The ability of the selected oligosaccharide composition as produced by a process as described in Examples 3-4, 12 and 13 is assessed for its ability to treat patients with ulcerative colitis (UC) in a Phase 2A, randomized, double-blind, controlled, multicenter study to evaluate the safety and efficacy of the selected oligosaccharide composition in participants with mild to moderately active Ulcerative Colitis. UC activity is determined by a Modified Mayo Score (MMS) of 3 to 7 (including MMS of 3 to 4 as mild and 5 to 7 as moderate). Enrollment of participants with an MMS 3 to 4 (mild) is limited to approximately 30% of the total study population unless the EMS is 2. A fecal calprotectin (FC) level of greater than or equal to 170 μg/g is required before randomization. Up to 90 participants are enrolled.

The study is conducted in 2 stages. After completion of the Screening Period, participants randomized to Stage 1 receive the high dose of selected oligosaccharide composition or control in a dose escalating fashion over the first 2 weeks to reach maximum dosing for the remaining 8 weeks. An interim analysis (IA) is planned after ˜45 participants complete treatment in Stage 1. Stage 2 enrollment begins immediately once Stage 1 enrollment has completed. The results of the IA determine if enrollment continues to the full 90 participants or if the study is stopped. Participants randomized in Stage 2 receive the low dose of selected oligosaccharide composition or control in a dose escalating fashion over the first 2 weeks to reach maximum dosing for the remaining 8 weeks.

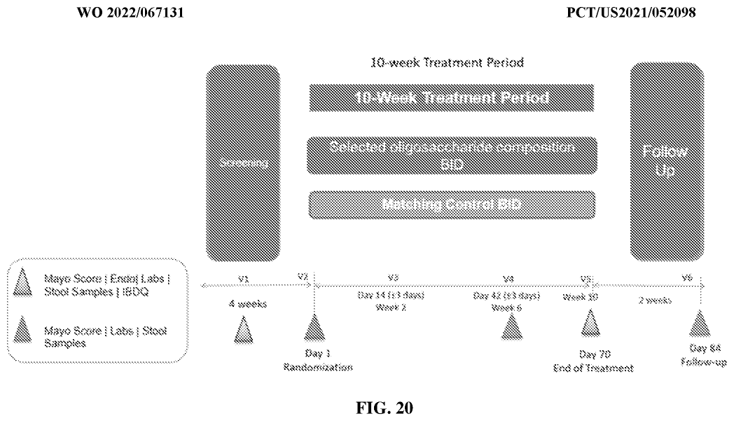

Screening Period: FIG. 20 provides an overview of the study design. Participants sign the informed consent form and complete screening assessments at Days-28 to Day 0. Participants are asked to provide a first morning stool sample for assessment of fecal calprotectin, fecal metabolites, and the microbiome. Blood samples are collected for baseline laboratory assessments. Participants will record daily stool frequency and rectal bleeding. Endoscopy is performed for a centrally read Endoscopic Mayo Subscore (EMS) and recording of a Modified and Total Mayo Score (MMS/TMS). Eligible participants per MMS of 3 to 7 are randomized into the trial. Central reading of histology is also be obtained in participants who are eligible for randomization. Participants are allowed no more than 28 days for completion of the Screening Period.

Treatment Period: At Day 1, participants have blood drawn for serologic biomarker assessments, and first morning FC stool sample are collected. Participants are instructed to complete the IBDQ and a 2-component Mayo Score (i.e., stool frequency subscore and rectal bleeding subscore) using the e-diary. Participants are randomized and administered the first dose of study drug at the site under supervision and then released to continue self-administration of study drug orally twice daily (BID) at home for 10 weeks. Study assessments at the subsequent visits during the Treatment period will include Mayo Score (with Physician's Global Assessment at baseline and end of treatment), IBDQ, collection of blood samples for laboratory analysis and first morning FC stool sample collection and microbiome stool sample collection. Endoscopy for central readings of EMS and histology are performed at the EOT visit or within 1 week before EOT. Participants who discontinue from the study early are not be replaced.

The selected oligosaccharide composition is supplied as a powder (9 g sachets) for reconstitution in 120 mL water, taken orally according to the following titration schedule:

### Stage 1:

High Dose of selected oligosaccharide composition (escalation to 36 g BID)
   Days 1 to 7: selected oligosaccharide composition administered as 9 g BID
   Days 8 to 14: selected oligosaccharide composition administered as 18 g BID
   Days 15 to 70: selected oligosaccharide composition administered as 36 g BID

### Stage 2:

Low Dose of selected oligosaccharide composition (escalation to 18 g BID)
   Days 1 to 7: selected oligosaccharide composition administered as 9 g BID
   Days 8 to 70: selected oligosaccharide composition administered as 18 g BID

Follow-up Visit: Two weeks after the EOT visit, a total of 3 first morning stool samples are collected over the 5 days before the visit; blood samples are collected at the visit for serologic biomarker assessments.

Inclusion criteria include the following:
Be male or female, ≥18 years and ≤75 years of age at the time of randomization with UC for at least 3 months prior to screening.
Be willing and able to provide informed consent.
Have a body mass index ≥17 and <40 kg/m2.
Have mild to moderate UC as evidenced by a Modified Mayo Score (MMS) of 3 to 7, an EMS of 1 or 2, and Bleeding Mayo Subscore (BMS)>1. Limit EMS of 1 to 30% and limit the MMS of 3 or 4 to 30% unless the EMS is 2.
Have screening endoscopy performed within 28 days prior to randomization.
UC Medication Prior to Screening:
   If not currently being treated with UC medication: Have not taken any UC medications for 28 days prior to screening.
   If currently being treated with UC medication: Have a stable regimen of UC medication for 28 days prior to screening. UC regimen cannot include budesonide >9 mg daily or prednisone >10 mg daily (or equivalent corticosteroid). See full list of exclusionary meds.

Participants must be willing to continue taking the same regimen of any current supplements and vitamins for the duration of the study.

Have fecal calprotectin >170 μg/g (to be determined at screening V1).

Exclusion criteria include the following: History of Crohn's Disease, indeterminate disease, or microscopic colitis.

Inflammatory bowel disease related to other etiologies, eg, sexually transmitted infections, anal trauma, Salmonella and/or Shigella infections.

History of UC restricted to 15 cm above the anal verge.

Known stool tests positive for ova and/or parasites or pathogenic stool culture within the 30 days before screening.

Participants must have a negative PCR stool test for Clostridium difficile. Participants with a positive PCR test may be retested once using stool culture.

Participants with known active COVID-19 infection or complications that could interfere with the trial

Major intra-abdominal surgery related to the bowel within 24 weeks prior to the Screening V1 and/or planned invasive surgery/hospitalization/procedures during the study (including scheduled lower endoscopies during the study period).

Participants with known or suspected toxic megacolon or small intestinal bacterial overgrowth

Participants with past medical history of diverticulitis, gastric bypass, colostomies, colectomies, ileal pouch anal anastomosis (IPAA), or previous intestinal surgery. Participants who have had minimally invasive surgeries (such as cholecystectomy or appendectomy) may be enrolled at principal investigator (PI) discretion.

Biologics or small molecules (eg, infliximab, adalimumab, golimumab, certolizumab, vedolizumab, ustekinumab, natalizumab, Janus kinase/signal transducer and activator of transcription [Jak-Stat] inhibitors, and sphingosine-1-phosphate [SIP] agonists) used within 24 weeks prior to screening.

Unable to discontinue steroid enemas or suppositories or mesalamine enemas or suppositories before the screening visit.

Received a fecal microbiota transplantation (FMT) within the last 24 weeks prior to screening.

Known allergy or intolerance to food products containing the control ingredients (erythritol) or ingredients of the selected oligosaccharide composition (eg, galactose); for example, participants with galactosemia.

Use of any antidiarrheal medications within 7 days prior to screening. Participants who fail this entry criterion can be rescreened ≥7 days after last antidiarrheal medication use

Systemic antibiotic treatment (oral or injectable) within 28 days prior to screening Participants with current elevated alanine transaminase (ALT) or aspartate transaminase (AST) >2.5 times upper limit of normal (ULN), and/or total bilirubin >1.5 times ULN.

Prior diagnosis of any cardiovascular, renal, hepatic, endocrine, infectious, hematological, oncologic, neuro-psychiatric or immune-mediated disorder, which, in the opinion of the PI, might impact the participant's safety or compliance, or the interpretation of the study results. If the participant's Type 2 diabetes has been controlled in the investigator's judgment and not subject to a symptomatic hyperglycemic or hypoglycemic event and non-insulin medication has been stable over the previous 12 weeks, then they can be considered for inclusion.

Treatment with any other investigational drugs within 28 days prior to the screening visit or 5 half-lives, whichever is longer.

Primary Endpoint included:
Change from baseline at Week 10 in fecal calprotectin (FC) in fecal samples of patients.

Secondary endpoints include:
Number of participants experiencing treatment-emergent adverse events (TEAEs)
Number of participants experiencing treatment-related adverse events (TRAEs)
Number of participants with treatment discontinuations due to TRAEs
Change from baseline in vital signs, safety laboratory analysis, physical examinations

Exploratory endpoints include:
Proportion of participants in remission using the individual components and combinations of the components of the Total Mayo Score
Change from baseline at Week 10 in Modified Mayo Score (MMS). The MMS are comprised of the Stool Frequency Subscore (SMS), the Rectal Bleeding Subscore (BMS), and the Endoscopic Mayo Subscore (EMS) only.
Change from baseline at Week 10 in Geboes Index
   Change from baseline in the 32-item Inflammatory Bowel Disease Questionnaire (IBDQ-32)

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1. An oligosaccharide composition comprising a plurality of oligosaccharides, the plurality of oligosaccharides being characterized by a multiplicity-edited gradient-enhanced ¹H-¹³C heteronuclear single quantum correlation (HSQC) NMR spectrum comprising one or more of signals 2, 3, and 11 of the following table,
wherein the area under the curve (AUC) for each of signals 1-11 is determined by obtaining the integration of integral regions defined by an 1H center position and an ¹³C center position using an elliptical shape, and wherein the spectrum is generated using a sample of the oligosaccharide composition having less than 2% monomer:

| | Center Position | | |
|---|---|---|---|
| | (ppm) | | Area under the curve (AUC) |
| Signal | ¹H | ¹³C | (% of total areas of signals 1-11) |
| 1 | 5.04 | 108.38 | 2.05-7.82 |
| 2 | 5.02 | 101.68 | 0.34-2.01 |
| 3 | 4.98 | 98.99 | 7.28-25.71 |
| 4 | 4.81 | 101.2 | 0.29-2.32 |
| 5 | 4.6 | 104.96 | 0.23-3.87 |
| 6 | 4.45 | 103.84 | 6.52-15.87 |
| 7 | 4.19 | 82.1 | 3.61-5.43 |
| 8 | 4.14 | 76.91 | 6.03-15.36 |
| 9 | 4.11 | 81.72 | 11.13-20.24 |
| 10 | 4.06 | 77.25 | 13.96-28.58 |
| 11 | 3.98 | 83.52 | 7.93-12.69 |

2. The oligosaccharide composition of claim 1, comprising 2 or 3 of signals 2, 3, and 11, wherein signal 2 has an AUC (% of total areas of signals 1-11) in the range of 0.34-2.01, signal 3 has an AUC (% of total areas of signals 1-11) in the range of 7.28-25.71, and signal 11 has an AUC (% of total areas of signals 1-11) in the range of 7.93-12.69.

3.-15. (canceled)

16. An oligosaccharide composition comprising a plurality of oligosaccharides, each oligosaccharide comprising a plurality of monomer radicals;
the plurality of oligosaccharides comprising one or more of the following monomer radicals:
(4) 3-galactopyranose monoradicals, representing 5.31-7.15 mol % of monomer radicals in the plurality of oligosaccharides;
(10) 6-galactopyranose monoradicals, representing 13.81-19.02 mol % of monomer radicals in the plurality of oligosaccharides;
(16) 3,6-galactopyranose and/or 2,6-galactofuranose diradicals, representing 4.14-5.93 mol % of monomer radicals in the plurality of oligosaccharides; and/or
(17) 2,6-galactopyranose diradicals, representing 1.98-2.99 mol % of monomer radicals in the plurality of oligosaccharides.

17.-19. (canceled)

20. The oligosaccharide composition of claim 16, wherein the plurality of oligosaccharides comprise each of the monomer radicals selected from radicals (1)-(20):
(1) t-galactofuranose monoradicals, representing 6.29-12.84 mol % of monomer radicals in the plurality of oligosaccharides;
(2) t-galactopyranose monoradicals, representing 20.45-28.28 mol % of monomer radicals in the plurality of oligosaccharides;
(3) 2-galactofuranose and/or 2-glucofuranose monoradicals, representing 2.73-3.46% mol % of monomer radicals in the plurality of oligosaccharides;
(4) 3-galactopyranose monoradicals, representing 5.31-7.15 mol % of monomer radicals in the plurality of oligosaccharides
(5) 3-galactofuranose monoradicals, representing 3.36-4.28 mol % of monomer radicals in the plurality of oligosaccharides:
(6) 2-galactopyranose monoradicals, representing 4.12-4.45 mol % of monomer radicals in the plurality of oligosaccharides;
(7) 4-galactopyranose and/or 5-galactofuranose monoradicals, representing 4.65-5.87 mol % of monomer radicals in the plurality of oligosaccharides;
(8) 2,3-galactofuranose diradicals, representing 0.43-0.82 mol % of monomer radicals in the plurality of oligosaccharides;
(9) 6-galactofuranose monoradicals, representing 2.92-9.58 mol % of monomer radicals in the plurality of oligosaccharides;
(10) 6-galactopyranose monoradicals, representing 13.81-19.02 mol % of monomer radicals in the plurality of oligosaccharides;
(11) 3,4-galactopyranose and/or 3,5-galactofuranose and/or 2,3-galactopyranose diradicals, representing 1.41-1.99 mol % of monomer radicals in the plurality of oligosaccharides;
(12) 2,4-glucopyranose and/or 2,5-glucofuranose and/or 2,4-galactopyranose and/or 2,5-galactofuranose diradicals, representing 0.88-1.21 mol % of monomer radicals in the plurality of oligosaccharides;
(13) 2,3,4-galactopyranose and/or 2,3,5-galactofuranose triradicals, representing 0.14-0.28 mol % of monomer radicals in the plurality of oligosaccharides;
(14) 3,6-galactofuranose diradicals, representing 1.69-2.27 mol % of monomer radicals in the plurality of oligosaccharides;
(15) 4,6-galactopyranose and/or 5,6-galactofuranose diradicals, representing 3.93-5.26 mol % of monomer radicals in the plurality of oligosaccharides;
(16) 3,6-galactopyranose and/or 2,6-galactofuranose diradicals, representing 4.14-5.93 mol % of monomer radicals in the plurality of oligosaccharides; and/or
(17) 2,6-galactopyranose diradicals, representing 1.98-2.99 mol % of monomer radicals in the plurality of oligosaccharides;
(18) 3,4,6-galactopyranose and/or 3,5,6-galactofuranose and/or 2,3,6-galactofuranose triradicals, representing 0.91-1.68 mol % of monomer radicals in the plurality of oligosaccharides;
(19) 2,3,6-galactopyranose and/or 2,4,6-galactopyranose and/or 2,5,6-galactofuranose triradicals, representing 0.01-3.10 mol % of monomer radicals in the plurality of oligosaccharides; and/or
(20) 2,3,4,6-galactopyranose and/or 2,3,5,6-galactofuranose quadradicals, representing 0.01-0.28 mol % of monomer radicals in the plurality of oligosaccharides.

21. The oligosaccharide composition of claim 20, wherein each oligosaccharide comprising a plurality of monomer radicals;
the plurality of oligosaccharides comprising one or more of the following monomer radicals:
(4) 3-galactopyranose monoradicals, representing 4.79-7.75 mol % of monomer radicals in the plurality of oligosaccharides;
(10) 6-galactopyranose monoradicals, representing 11.64-22.24 mol % of monomer radicals in the plurality of oligosaccharides;
(16) 3,6-galactopyranose and/or 2,6-galactofuranose diradicals, representing 2.20-7.06 mol % of monomer radicals in the plurality of oligosaccharides; and/or
(17) 2,6-galactopyranose diradicals, representing 0.89-3.63 mol % of monomer radicals in the plurality of oligosaccharides.

22.-57. (canceled)

58. A method of reducing inflammation in a subject, the method comprising administering to the gastrointestinal tract of the subject an effective amount of an oligosaccharide composition according to claim 1.

59. (canceled)

60. A method of treating a subject having or suspected of having an inflammatory and immune disorder, the method comprising administering to the gastrointestinal tract of the subject an effective amount of an oligosaccharide composition according to claim 1, thereby treating the subject.

61. A method of treating a subject having or suspected of having an inflammatory and immune disorder, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I): 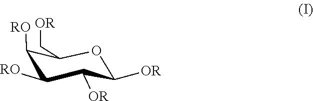
wherein R in Formula (I) is independently selected from hydrogen, and Formulae (Ia), (Ib), (Ic), (Id): 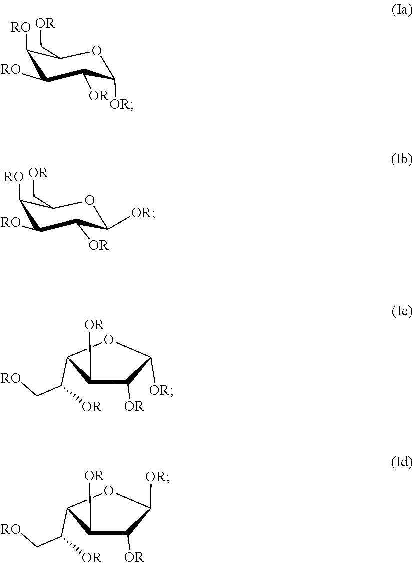
wherein R in Formulae (Ia), (Ib), (Ic), and (Id) is independently defined as above in Formula (I), thereby treating the subject.

62.-75. (canceled)

76. A method of treating a subject having or suspected of having an inflammatory bowel disease, the method comprising administering to the gastrointestinal tract of the subject an effective amount of an oligosaccharide composition according to claim 1, thereby treating the subject.

77. A method of treating a subject having or suspected of having an inflammatory bowel disease, the method comprising administering to the gastrointestinal tract of the subject an effective amount of an oligosaccharide composition, wherein the oligosaccharide composition has an average degree of polymerization of 5-20 and comprises a plurality of oligosaccharides selected from Formula (I): 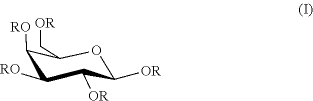
wherein R in Formula (I) is independently selected from hydrogen, and Formulae (Ia), (Ib), (Ic), (Id): 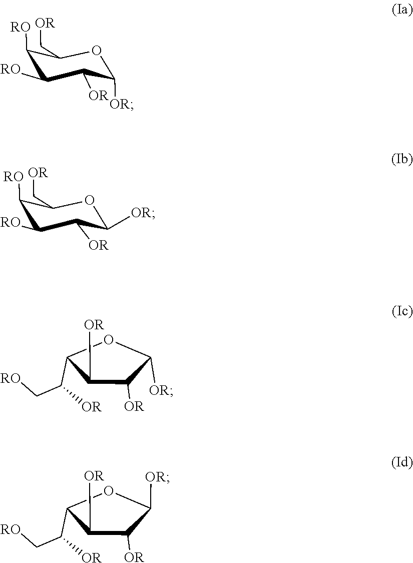
wherein R in Formulae (Ia), (Ib), (Ic), and (Id) is independently defined as above in Formula (I), thereby treating the subject.

78.-89. (canceled)

90. A method of increasing the relative or absolute abundance of short chain fatty acids in a subject, the method comprising administering to the gastrointestinal tract of the subject an effective amount of an oligosaccharide composition according to claim 1.

91. The method of claim 90, wherein the relative or absolute abundance of short chain fatty acids is increased by at least 5%, 10%, 20%, or 30%, compared to a baseline measurement (e.g., wherein the baseline measurement is determined prior to treatment).

92. The method of 90, wherein the short chain fatty acids are butyrate, acetate, and/or propionate.

93. A method of decreasing the relative or absolute abundance of pro-inflammatory and/or pathogenic bacteria in a subject, the method comprising administering to the gastrointestinal tract of the subject an effective amount of an oligosaccharide composition according to claim 1.

94. The method of claim 93, wherein the pro-inflammatory and/or pathogenic bacteria are Enterobacteriaceae and/or Ruminococcaceae.

95. A method of increasing the relative or absolute abundance of commensal bacteria in a subject, the method comprising administering to the gastrointestinal tract of the subject an effective amount of an oligosaccharide composition according to claim 1.

96. The method of claim 95, wherein the commensal bacteria are Parabacteroides and/or Bacteroides.

97.-126. (canceled)

127. A method of decreasing the levels of one or more biomarkers associated with inflammation in a subject, optionally a subject exhibiting an inflammatory disease, comprising administering the oligosaccharide composition of claim 1 to the subject in an effective amount to decrease levels of the one or more biomarkers, relative to a baseline measurement.

128. (canceled)

129. A method of decreasing the abundance of one or more pathobionts in a subject, optionally a subject exhibiting an inflammatory disease, comprising administering the oligosaccharide composition of claim 1 to the subject in an effective amount to decrease the abundance of the one or more pathobionts.

130. (canceled)

131. A method of increasing the abundance of one or more commensal taxa in a subject, optionally a subject exhibiting an inflammatory disease, comprising administering the oligosaccharide composition of claim 1 to the subject in an effective amount to increase the abundance of the one or more commensal taxa.

132. (canceled)
